(19) United States
(12) Patent Application Publication
(10) Pub. No.: US 2026/0297027 A1
(43) Pub. Date: Oct. 1, 2026

(54) Non-Peptide Small Molecule FGF14:Nav1.6 Channel Complex Protein-Protein Interaction Modulators

(71) Applicant: The Board of Regents of The University of Texas System, Austin, TX (US)

(72) Inventors: Jia Zhou, Galveston, TX (US); Fernanda Laezza, Galveston, TX (US); Pingyuan Wang, Galveston, TX (US); Nolan M. Dvorak, Galveston, TX (US); Paul A. Wadsworth, Galveston, TX (US)

(21) Appl. No.: 19/490,041

(22) PCT Filed: Jun. 5, 2024
(86) PCT No.: PCT/US2024/032668

§ 371 Date: Dec. 4, 2025

Related U.S. Application Data
(60) Provisional application No. 63/506,356, filed on Jun. 5, 2023.

(51) Int. Cl. C07D 209/12; A61K 31/405; A61K 31/422; A61K 31/427; A61K 31/437; A61K 31/4439; A61K 31/496; A61K 31/5377; A61K 31/675; C07D 209/14; C07D 209/42; C07D 401/12; C07D 405/12; C07D 405/14; C07D 413/12; C07D 417/12; C07D 471/04; C07F 9/572

(52) U.S. Cl. CPC C07D 209/12; A61K 31/405; A61K 31/422; A61K 31/427; A61K 31/437; A61K 31/4439; A61K 31/496; A61K 31/5377; A61K 31/675; C07D 209/14; C07D 209/42; C07D 401/12; C07D 405/12; C07D 405/14; C07D 413/12; C07D 417/12; C07D 471/04; C07F 9/5728

(57) ABSTRACT

The present invention relates to novel small molecule FGF14:NAV1.6 channel complex protein-protein interaction modulators of the general structure according to Formula (I), and the preparation and use thereof:

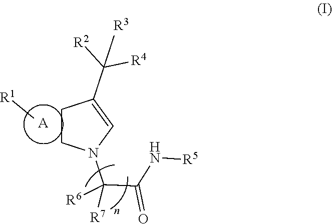

### CROSS REFERENCE TO RELATED APPLICATIONS

This application claims priority to U.S. Application No. 63/506,356, filed on Jun. 5, 2023, the contents of which are hereby incorporated by reference in its entirety.

### STATEMENT OF FEDERALLY SPONSORED RESEARCH AND DEVELOPMENT

This invention was made with government support under NIH/NIMH Grant No. R01MH111107 awarded by the National Institutes of Health (NIH) and National Institute of Mental Health (NIMH). The government has certain rights in the invention.

### FIELD OF THE INVENTION

The field of the invention relates generally to modulators of FGF14:NAV1.6 channel complex protein-protein interaction. More specifically, the field of invention relates to non-peptide small molecule modulators of FGF14:NAV1.6 channel complex protein-protein interaction modulators.

### BACKGROUND

This background information is provided for the purpose of making information believed by the applicant to be of possible relevance to the present invention. No admission is necessarily intended, nor should it be construed, that any of the information disclosed herein constitutes prior art against the present invention.

### DESCRIPTION

One aspect of the invention pertains to a compound of Formula I or a pharmaceutically acceptable salt thereof, wherein:

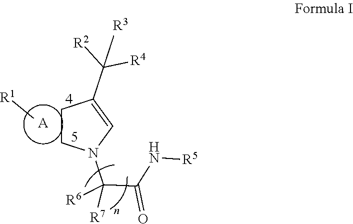

R¹ is H, alkyl, alkoxy, halogen, cyano, amino, hydroxyl, NO₂, CF₃ or —OCF₃;
A is an aryl ring or a heteroaryl ring (e.g., pyridine), wherein ring A is fused to the
C4 and C5 of the 5-membered N-heterocycle ring moiety of Formula I, e.g., as shown the compounds exemplified below;
R² is H, alkyl, alkoxy, halogen, —CO₂R¹⁰, —CO₂Me or hydroxyl;
R³ is H, alkyl, alkoxy, halogen or hydroxyl;
   or R² and R³ together form a 3-6 membered cycloalkyl ring;
R⁴ is H, alkyl, alkoxy, halogen, cyano, hydroxyl or NT¹T²;
T¹ is H, alkyl;
T² is independently chosen from: H, alkyl, cycloalkyl, benzyl, allyl, hydroxyl-alkyl;
   or T¹ and T² together form a 4-12 membered cycloalkyl ring or cycloheteroalkyl ring, wherein the 4-12 membered cycloalkyl ring or cycloheteroalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
n=0, 1, 2, 3, or 4;
R⁵ is H, alkyl, aryl, cycloalkyl (e.g., is a bridged 6-14-membered bicyclic cycloalkyl, with 1-3 carbon length “bridge”); or heteroaryl, wherein each ring is optionally substituted with one or more groups selected independently from: H, alkyl, cycloalkyl, alkenyl, aryl, heteroaryl, benzyl, alkoxy (e.g., OMe, 3-OMe), halogen, cyano (e.g., 3-CN), nitro, amino, amido (e.g., 3-amido hydroxyl, —COOR⁸ (e.g., —COOMe), —CONHR⁹, CHF₂, CF₃ or —OCF₃, wherein alkyl is optionally substituted with one or more substituents chosen from: hydroxyl, cyan, amino, or halogen;
R⁶, and R⁷ are independently chosen from H, alkyl, F, CHF₂, CF₃, etc., or R⁶ and R⁷ taken together form a 3-7 membered ring (e.g., cyclopropane);
R⁸ is independently chosen from: H, alkyl, aryl, cycloalkyl, benzyl; and
R⁹ is independently chosen from: H, alkyl, aryl, cycloalkyl, benzyl, or substituted benzyl.
R¹⁰ is alkyl (e.g., C1-C6-alkyl), aryl, or alkylaryl.

Another aspect of the invention pertains to a compound of the Formula (II) or a pharmaceutically acceptable salt thereof, wherein:

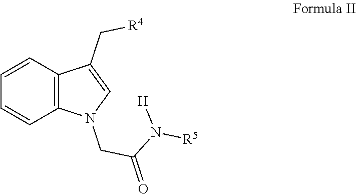

R⁴ is hydroxyl or NT¹T²; wherein T¹ is H or alkyl; and wherein T² is independently chosen from H, alkyl, cycloalkyl, benzyl, allyl, hydroxyl-alkyl;
or T¹ and T² together form a 4-12 membered cycloalkyl ring or cycloheteroalkyl ring, wherein the 4-12 membered cycloalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
R⁵ is an aryl ring or a cycloalkyl ring, wherein each ring is optionally substituted with one or more groups selected independently from: H, alkyl, cycloalkyl, alkenyl, aryl, heteroaryl, benzyl, alkoxy, halogen, cyano, nitro, amino, hydroxyl, —COOR⁸, —CONR⁹, CHF₂, CF₃ or —OCF₃, wherein alkyl is optionally substituted with one or more chosen substituents chosen from: hydroxyl, cyano, amino, or halogen;
R⁸ is independently chosen from: H, alkyl, aryl, cycloalkyl, or benzyl; and
R⁸ is independently chosen from: H, alkyl, aryl, cycloalkyl, benzyl, or substituted benzyl.

A further aspect of the invention pertains to a compound of Formula III, or a pharmaceutically acceptable salt thereof, wherein:

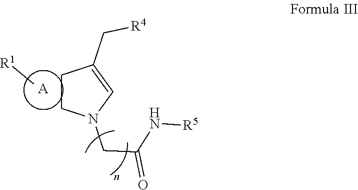

wherein A, n, R¹, R⁴, and R⁵ are as defined for the compound of Formula I.

A yet further aspect of the invention pertains to compound of Formula IV, or a pharmaceutically acceptable salt thereof, wherein:

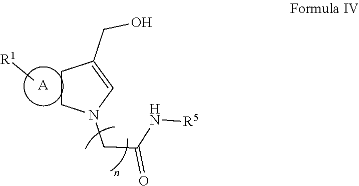

wherein A, n, R¹, and R⁵ are defined as for the compound of Formula I.

A yet further aspect of the invention pertains to compound of Formula V, or a pharmaceutically acceptable salt thereof, wherein:

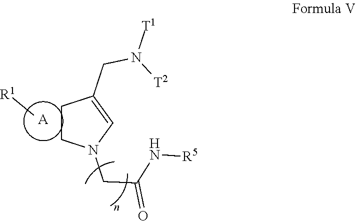

wherein A, n, R¹, R⁵, T¹, and T² are defined as for the compound of Formula I.

A yet further aspect of the invention pertains to a compound of Formula VI, or a pharmaceutically acceptable salt thereof, wherein:

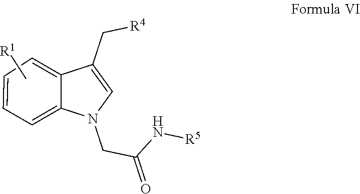

wherein R¹, R⁴, and R⁵ are defined as for the compound of Formula I.

A yet further aspect of the invention pertains to a compound of Formula VII, or a pharmaceutically acceptable salt thereof, wherein:

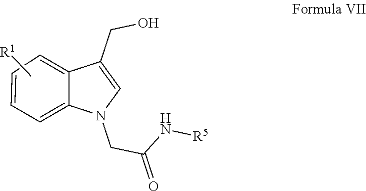

wherein R¹ and R⁵ are defined as for the compound of Formula I.

A yet further aspect of the invention pertains to a compound of Formula VIII, or a pharmaceutically acceptable salt thereof, wherein:

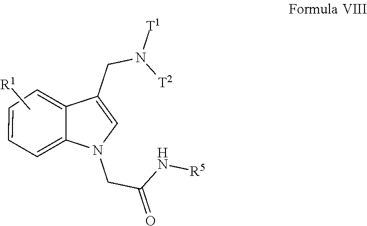

wherein R¹, R⁵, T¹, and T² are defined as for the compound of Formula I.

A yet further aspect of the invention pertains to a compound of Formula IX, or a pharmaceutically acceptable salt thereof, wherein:

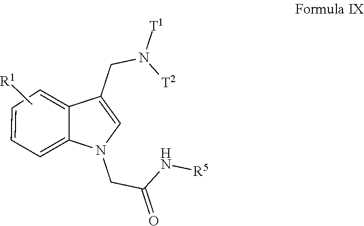

wherein R¹, R⁵, T¹, and T² are defined as for the compound of Formula I.

A yet further aspect of the invention pertains to a compound of Formula X, or a pharmaceutically acceptable salt thereof, wherein:

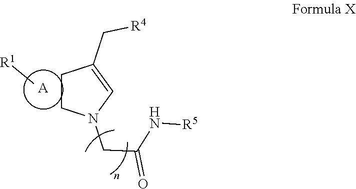

wherein A is a heteroaryl ring, n, R¹, R⁴ and R⁵ are defined as for the compound of Formula I.

A yet further aspect of the invention pertains to a method of treating a disease or condition in a patient comprising administering to the patient a therapeutically effective amount of a compound of disclosed herein or a pharmaceutically acceptable salt thereof. In some embodiments, treatment of said disease or condition involves modulating a FGF14:NAV1.6 channel complex protein-protein interaction.

All publications mentioned herein are incorporated by reference to the extent they support the present invention.

### BRIEF DESCRIPTION OF FIGURES

FIG. 1. High throughput screening against the FGF14:Nav1.6 complex. FIG. 1A. Summary of the LCA primary screen of ~45,000 compounds showing hits based on a Z-score criterion. FIG. 1B. Compounds are excluded based on the CellTiter Blue (CTB) cell viability fluorescence assay. FIG. 1C. Counter screen of non-toxic hits against the luciferase enzyme. FIG. 1D. Dose-response of repurchased top hits after triplicate rescreen and prioritization based on drug-like properties.

FIG. 2. Selectivity and orthogonal validation of hits. FIG. 2A. The top 16 hits were screened (50 μM) against the FGF13-1a:Nav1.6, FGF13-1B:Nav1.6, and FGF14:Nav1.2 complexes, as well as the FGF14 dimer, using LCA. Compounds that modulated complex assembly by ≥25% were excluded from further studies. FIG. 2B. The remaining 5 selective hits were then quantitatively assessed using SPR. Binding sensorgrams are shown for FGF14 (left) and Nav1.6 (middle) and normalized steady-state saturation plots (right) reveal differences in compound:protein binding affinities. Kinetic analysis of each ligand/analyte interaction was obtained by fitting the response data to the simplest Langmuir 1:1 interaction model (K_{D}=k_{off}/kₒₙ). The K_{D} of compound 7605 was 2.9 mM for FGF14 and 13.6 mM for Nav1.6.

FIG. 3. Screening of 50 analogs of 7605086 using the LCA. Single concentration screening of the first batch of 7605086 analogs (34 compounds total; concentration=5 uM) using the LCA.

FIG. 4A-FIG. 4H. Dose-responses of top compounds. Note that PW01028 displayed nanomolar potency in the LCA dose-response studies.

FIG. 5. Screening of an additional batch of analogs using the LCA. Single concentration screening of the first batch of analogs (16 compounds total; concentration=5 μM) using the LCA.

FIG. 6A-6D. Dose-responses of top compounds. Note that PW01028 displayed nanomolar potency in the LCA dose-response studies.

FIG. 7. Third generation of PW1028 analogs were synthesized and single concentration activity screening was tested.

FIG. 8. Dose-responses of top compound.

FIG. 9. The binding modes and molecular docking of compound PW01028. FIG. 9A. Docking of PW01028 (magenta) into the binding pocket of FGF14 in 3D view. Important residues are drawn in sticks. Hydrogen bonds are shown as dashed magenta lines, while π-π interactions are shown as dashed blue lines. FIG. 9B. Docking of compound PW01028 into the binding pocket of FGF14 in 2D view. Important residues and key interactions are presented in a similar fashion.

FIG. 10. Ligand binding studies of Compound PW01028. FIG. 10A. Dose-response curve of compound PW01028 as determined via in-cell testing using the LCA of Nav1.6 WT complexed with either FGF14WT (10A) or vs FGF14R117A (10B) (10B, 10C) Binding of compound PW01028 to FGF14 (10C, 10D), FGF14 R117A (10E) or the CTD of Naᵥ1.6 (10F).

FIG. 11. Compound PW01028 inhibits shifts voltage-dependence of steady-state inactivation of Na+ currents and increases firing in MSN in an-FGF14 dependent manner. FIG. 11A. Traces of I_{Na} peak currents (left column, asterisk) from cells treated with vehicle (0.1% DMSO; black) or compound PW01028 (1 μM; blue) elicited using the depicted voltage-clamp protocol (inset). The traces on the right represent a fraction of inactivated channels. FIG. 11B. Voltage-dependence of steady-state inactivation curves for the indicated experimental groups. Data were fitted using the Boltzmann equations. FIG. 11C. Comparison of V_{1/2} of steady-state inactivation between indicated groups. Data are mean±SEM (n=6-10 cells/group). Significance assessed using Student's t-test. (11D-11F). Effect of compound PW01028 on MSN firing from Fgf14^{+/+} mice and relative quantification. (n=19-20/group; ****, p<0.0001; Student's t-test). (FIG. 11G-11I). Lack of effect of compound PW01028 in MSN firing from Fgf14^{-/-} mice and relative quantification (n=7-8/group; p>0.05; Student's t-test. Data are mean+SEM.

FIG. 12. compound PW01028 inhibits shifts voltage-dependence of steady-state inactivation of Na+ currents and increases firing in MSN in an-FGF14 dependent manner. (FIG. 12A) Traces of I_{Na} peak currents (left column, asterisk) from cells treated with vehicle (0.1% DMSO; black) or compound PW01028 (1 μM; blue) elicited using the depicted voltage-clamp protocol (inset). The traces on the right represent a fraction of inactivated channels (FIG. 12B) Voltage-dependence of steady-state inactivation curves for the indicated experimental groups. Data were fitted using the Boltzmann equations. (FIG. 12C) Comparison of V_{1/2} of steady-state inactivation between indicated groups. Data are mean±SEM (n=6-10 cells/group). Significance assessed using Student's t-test. (FIG. 12D-12F). Effect of compound PW01028 on MSN firing from Fgf14^{+/+} mice and relative quantification. (n=19-20/group; ****, p<0.0001; Student's t-test). (FIG. 12G-12I). Lack of effect of compound PW01028 in MSN firing from Fgf14^{-/-} mice and relative quantification (n=7-8/group; p>0.05; Student's t-test. Data are mean+SEM.

### 1.0 DEFINITIONS

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated invention, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

The use of “or” means “and/or” unless stated otherwise.

The use of “a” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” refers to a ±10% variation from the nominal value. It is to be understood that such a variation is always included in any given value provided herein, whether or not it is specifically referred to.

The term “pharmaceutically acceptable salt” refers to those salts of the compounds of the present invention which are, within the scope of sound medical judgment, suitable for use in contact with the tissues of patients without undue toxicity, and the like. As used herein, the term “pharmaceutically acceptable salt” may include acetate, hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. (See S. M. Barge et al., “Pharmaceutical Salts,” J. Pharm. Sci., 66:1-19 (1977), which is incorporated herein by reference in its entirety, for further examples of pharmaceutically acceptable salt).

The term “HBTU” refers to 3-[Bis(dimethylamino)methyliumyl]-3H-benzotriazol-1-oxide hexafluorophosphate (also known as 2-(1H-benzotriazol-1-yl)-1,1,3,3-tetramethyluronium hexafluorophosphate).

The term “HOBt” refers the following structure, known as 1-hydroxybenzotriazole, (including hydrates and polymorphs, thereof):

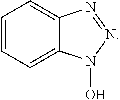

The term “DIEA” refers to N,N-Diisopropylethylamine (also known as Hünig's base, DIPEA, and ethyldiisopropylamine).

The term “DCM” refers to dichloromethane (also known as methylene chloride).

The term “TFA” refers to trifluoroacetic acid.

The term “rt” refers to room temperature.

The term “alkyl” as used herein by itself or as part of another group refers to both straight and branched chain radicals, and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The term “alkyl” may include methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl. Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g., fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

In certain embodiments, the term “cycloalkyl” includes bicyclic ring systems. The bicyclic ring system may be in the form of a bridged, fused, or spiro form.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O, and N. The heteroatom(s) may be placed at any interior position of the heteroalkyl group or at the position at which the alkyl group is attached to the remainder of the molecule. Up to two heteroatoms may be consecutive.

The term “alkylene” as used herein refers to straight and branched chain alkyl linking groups, i.e., an alkyl group that links one group to another group in a molecule. In some embodiments, the term “alkylene” may include —(CH₂)ₙ— where n is 2-8.

The term “aryl” means a polyunsaturated hydrocarbon substituent. Aryl groups can be monocyclic or polycyclic (e.g., 2 to 3 rings that are fused together or linked covalently). Non-limiting examples of aryl and heteroaryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

The term “heteroaryl” as used herein refers to groups having 5 to 14 ring atoms; 6, 10 or 14 7π-electrons shared in a cyclic array; and containing carbon atoms and 1, 2 or 3 oxygen, nitrogen or sulfur heteroatoms. A heteroaryl group can be attached to the remainder of the molecule through a carbon or heteroatom. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, 2-aminopyridine, 4-aminopyridine, 2-aminoimidazoline, and 4-aminoimidazoline.

An “amino” group refers to an —NH₂ group.

An “amido” group refers to an —CONH₂ group. An alkylamido group refers to an —CONHR group wherein R is as defined above. A dialkylamido group refers to an —CONRR′ group wherein R and R′ are as defined above.

The term “halogen” or “halo” as used herein by itself or as part of another group refers to chlorine, bromine, fluorine or iodine.

The term “hydroxy” or “hydroxyl” as used herein by itself or as part of another group refers to an OH group.

An “alkoxy” group refers to an —O-alkyl group wherein “alkyl” is as defined above. In one embodiment, the alkyl group has 1-12 carbons. In another embodiment, the alkyl group has 1-7 carbons. In a further embodiment, the alkyl group has 1-6 carbons. In another embodiment, the alkyl group has 1-4 carbons.

A “thio” group refers to an —SH group.

An “alkylthio” group refers to an —SR group wherein R is alkyl as defined above.

The term “heterocycle” or “heterocyclic ring”, as used herein except where noted, represents a stable 5- to 7-membered monocyclic-, or stable 7- to 11-membered bicyclic heterocyclic ring system, any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Rings may contain one oxygen or sulfur, one to three nitrogen atoms, or one oxygen or sulfur combined with one or two nitrogen atoms. The heterocyclic ring may be attached at any heteroatom or carbon atom that results in the creation of a stable structure.

The term “alkylamino” as used herein by itself or as part of another group refers to an amino group which is substituted with one alkyl group having from 1 to 6 carbon atoms. The term “dialkylamino” as used herein by itself or as part of another group refers to an amino group which is substituted with two alkyl groups, each having from 1 to 6 carbon atoms.

The term “arylamine” or “arylamino” as used herein by itself or as part of another group refers to an amino group which is substituted with an aryl group, as defined above.

As used herein, the term “arylalkyl” denotes an alkyl group substituted with an aryl group, for example, Ph-CH₂—, etc.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, alkyl, heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl (—C(O)NR₂), unsubstituted alkyl, unsubstituted heteroalkyl, alkoxy, alkylthio, alkylamino, (alkyl)₂amino, alkylsulfinyl, alkyl sulfonyl, aryl sulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄ alkyl, phenyl, benzyl, NH₂, —NH(C₁₋₄ alkyl), —N(c₁₋₄ alkyl)₂, —NO2, —S(C₁₋₄ alkyl), SO₂(C₁₋₄ alkyl), —CO₂(C₁₋₄ alkyl), and O(C₁₋₄ alkyl).

It is to be understood that both the foregoing descriptions are exemplary, and thus do not restrict the scope of the invention.

Psychiatric disorders are neural circuitry alterations that lead to malfunctioning in high-order psychological domains. To parse out disease complexity and advance treatment, new biological signatures that break free of traditional clinical classifications—spanning longitudinally across disorders in lieu of a single domain-disease view—are required. Within this research framework lies the need for new molecular tools to interrogate specific neural circuits. Protein-protein interactions (PPI) within ion channel complexes, fine-tune neuronal excitability and are emerging as links to the biology of psychiatric disorders and addiction. Their highly specific and flexible interfaces could make protein-channel interactions ideal targets for development of molecular probes. This would provide the neuropharmacology community with the tools to interrogate circuit biology with unprecedented precision and lay the foundation for transformative new therapeutic strategies. In medium spiny neurons (MSN), the principal cell type of the nucleus accumbens (NAc), the PPI between the voltage-gated Na⁺ (Naᵥ) channel isoform 1.6 (Naᵥ1.6) and fibroblast growth factor 14 (FGF14) is crucial to action potential firing. Given that MSNs provide the sole output of the NAc, FGF14:Naᵥ1.6 complex assembly is critical to regulate the firing rate of NAc targets by controlling GABA release onto the ventral tegmental area and ventral pallidum via the dopaminergic receptors type 1 direct (D1) and type 2 indirect (D2) pathways, respectively. As these pathways regulate motivation, anhedonia, and reward-related behaviors, probes that modulate MSN activity by fine-tuning FGF14:Naᵥ1.6 complex assembly would be valuable for interrogating the circuitry affected in many neuropsychiatric disorders.

Novel probes are needed that serve as precise in vivo measures of mechanisms hypothesized to underlie circuit deficits in mental disorders. Probes developed from molecular knowledge will provide the foundation for a paradigm shift in neuropsychopharmacology, leading to rigorous mechanistic hypotheses and future medications.

The nucleus accumbens (NAc), located within the ventral striatum, serves as the central point of brain reward circuitry, controlling goal-directed behaviors and motivation for reward for natural stimuli and drug of abuse. Within the NIMH Research Domain Criteria (RDoC), motivation is a fundamental dimension of behavior that cuts across diagnostic boundaries. Lack of motivation is a prevalent symptom in a number of neuropsychiatric illnesses such as schizophrenia, major depressive disorder, and neurodevelopmental disorders. It is therefore critical to develop adequate molecular tools to probe the circuits and the behaviors associated with motivation. MSNs comprise 95% of the NAc neuronal population, are formed exclusively by GABAergic cells, and give rise to the only output of the NAc. Recent studies in rodent models have mechanistically linked MSN activity with reward-expectation and motivation for reward showing that optogenetic inhibition of MSN firing disrupts food-incentive goal-directed behaviors. Thus, it is hypothesized that probes targeting the FGF14:Naᵥ1.6 complex will provide novel tools to interrogate molecular, cellular, and circuital mechanisms underlying motivated behaviors with a broad impact for psychiatry. The fine-tuned regulation of neuronal firing relies on the integrity of ion channel macromolecular complexes. These complexes are formed by a nexus of tightly regulated PPI that define the channel functional phenotype. In previous studies, FGF14 was established as a functionally relevant component of the Nav1.6 channel complex and a primary determinant of excitability in MSNs. Genetic silencing of either FGF14 or Nav1.6 affects MSN firing and plasticity in the NAc, and leads to changes in motivated behaviors associated with depression, anxiety, addiction, schizophrenia and psychosis, thereby demonstrating a link between the FGF14:Nav1.6 complex and a full range affective disorders.

Through binding to the intracellular C-terminal tail of Nav1.6, FGF14 controls Nav1.6 gating and inactivation, and axonal targeting in neurons, properties conferred by a high degree of structural and functional specificity of the PPI interface. Evidence for druggability of this PPI interface provided by the use of short peptides and peptidomimetics has spurred interest in running a high-throughput screening (HTS) campaign aimed at identifying small molecule modulators of the FGF14:Nav1.6 complex.

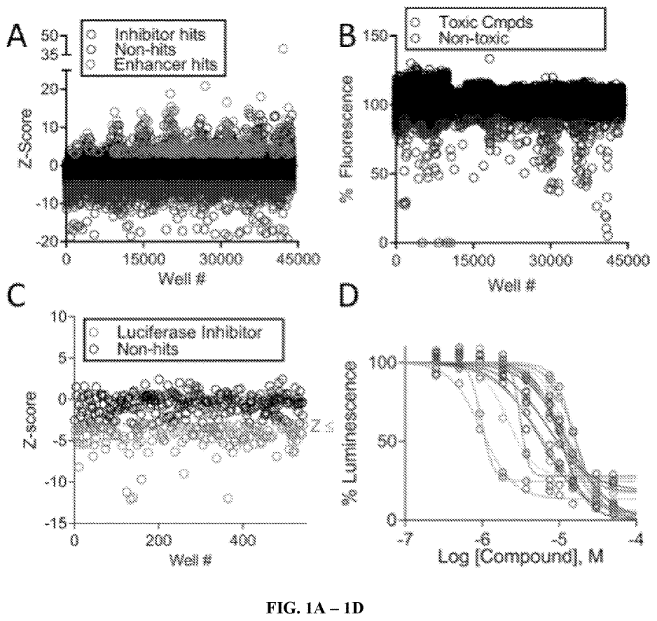

To identify small molecule probes targeting the FGF14:Nav1.6 PPI interface, the FGF14:Nav1.6 complex was reconstituted using the split-luciferase complementation assay (LCA), and a candidate pool of ~45,000 commercially available compounds was screened. An established screening pipeline was employed to validate hits. FIG. 1A. shows results of the LCA primary screen of ~45,000 compounds showing hits based on a Z-score criterion. FIG. 1B summarizes exclusion of compounds based on the CellTiter Blue (CTB) cell viability fluorescence assay. FIG. 1C shows results of counter screen of non-toxic hits against the luciferase enzyme. FIG. 1D shows dose-response of repurchased top hits after triplicate rescreen and prioritization based on drug-like properties.

Of the three identified hits with conserved pharmacodynamic profiles, compound 7605086 displayed the most favorable drug-like properties for CNS drug development. To improve the potency and aqueous solubility of compound 7605086, substituents of the parental scaffold were replaced, producing probe compound PW01028 after two rounds of optimization.

TABLE 1
Efficacy of 34 1^{st} generation 7605086 analogs.

| Cmp ID | Efficacy (%) |
|---|---|
| PW0999 | 5.75 |
| PW1001 | 1.2 |
| PW1002 | 0.5 |
| PW1003 | 10 |
| PW1004 | 8.025 |
| PW1005 | 4.525 |
| PW1006 | 5.875 |
| PW1007 | 32.95 |
| PW1011 | 4.1 |
| PW1016 | 1.025 |
| PW1017 | 4.325 |
| PW1020 | 5.775 |
| PW1022 | 37.85 |
| PW1023 | 28.35 |
| PW1024 | 67.725 |
| PW1025 | 43.75 |
| PW1026 | 70.1 |
| PW1027 | 54.2 |
| PW1028 | 62.825 |
| PW1029 | 28.825 |
| PW1030 | 51.725 |
| PW1031 | 55.075 |
| PW1032 | 3.45 |
| PW1033 | 19.725 |
| PW1034 | 68.525 |
| PW1035 | 58.425 |
| PW1036 | 44.6 |
| PW1037 | 18.475 |
| PW1038 | 25.85 |
| PW1039 | 37.425 |
| PW1040 | 13.225 |
| PW1041 | 1.875 |
| PW1042 | 26.85 |
| PW9100 | 4.975 |

Notably, according to the LCA, compound PW01028 modulated FGF14:Nav1.6 complex formation with an IC₅₀ of ~250 nM. In addition, surface plasmon resonance (SPR) showed that compound PW01028 displayed nanomolar binding affinity to FGF14 with evidence for ligand binding to the FGF14^{R117} residue, a previously identified “hot-spot” at the FGF14:Nav1.6 PPI interface. Functionally, compound PW01028 was shown to exacerbate FGF14-mediated regulatory effects on Nav1.6 channel inactivation, causing a marked (+15 mV) depolarizing shift in the voltage-dependence of steady-state inactivation of the transient Na+ current. The mechanism of action (MOA) of compound PW01028 was recapitulated in MSNs of the NAc, where the compound hampered Nav channel closure, prolonged Na+ currents, and consequently potentiated intrinsic firing. Importantly, these phenotypes observed in wild-type mice were absent in Fgf14^{-/-} mice. This suggested that the compound regulates accumbal output to increase motivational states in demotivated subjects.

TABLE 2
Efficacy of certain embodiments, namely exemplary analogs.

| Compound ID | Efficacy (%) |
|---|---|
| PW1083 | 11.10899 |
| PW1073 | 55.83746 |
| PW1078 | 7.966694 |
| PW1074 | 5.11128 |
| PW1084 | 1.700025 |
| PW1076 | 24.84255 |
| PW1072 | 24.57135 |
| PW1062 | 13.31443 |
| PW1051 | 68.0178 |
| PW1053 | 13.15754 |
| PW1050 | 65.4728 |
| PW1061 | 14.72645 |
| PW1046 | 5.414976 |
| PW1045 | 9.004415 |
| PW1044 | 13.20797 |
| PW1043 | 9.505345 |

TABLE 3
Efficacy of certain embodiments, namely exemplary analogs.

| Compound ID | Efficacy (%) |
|---|---|
| JY0142 | 42.83943 |
| JY0149 | 6.9265 |
| JY0170 | 23.22837 |
| JY0143 | 35.56067 |
| JY0186 | 5.203733 |
| JY0160 | 22.37417 |
| JY0162 | 1.198267 |
| JY0151 | 23.40067 |
| JY0145 | 4.313633 |
| JY0161 | 16.2152 |
| JY0153 | 3.646033 |
| JY0163 | 0.3953 |
| JY0164 | 1.399233 |
| JY0166 | 13.39413 |
| JY0147 | 11.5709 |
| JY0155 | 52.93433 |
| JY0144 | 22.10757 |
| JY0156 | 27.38817 |
| JY0188 | 20.18383 |
| JY0159 | 18.87533 |
| JY0154 | 46.89047 |
| JY0168 | 37.739 |
| JY0169 | 27.1623 |
| JY0189 | 28.167 |
| JY0137 | 32.23257 |
| JY0167 | 20.9938 |
| JY0187 | 12.4888 |
| JY0172 | 40.41823 |

### EXAMPLES

### Biological Testing

Primary assays: Split-luciferase complementation assay (LCA). The primary assay to detect FGF14:Nav1.6 complex formation in real-time in living cells is the split-luciferase complementation assay (LCA), which has been designed and successfully implemented specifically to study PPI between the Nav channel and iFGFs. With LCA, in-cell reconstitution of the FGF14:Nav1.6 channel complex was detected upon addition of the D-luciferin substrate, as a robust light signal representing the strength of interaction between the two binding partners. LCA is a highly sensitive, robust, and reproducible assay tolerable to non-aqueous solvents such as DMSO to a final concentration of 0.5%. Compounds were first tested at one single concentration in 384-well plates. Statistical parameters for compound selection are as follows: Z′=0.75, signal to noise=14.8, signal window=11.3. The high signal-to-noise ratio, favorable dynamic range, and reversibility of luminescence-based signals make LCA the ideal for HTS of potential regulators, as well as rapid dose-dependency studies (≥8 concentrations) to help guide subsequent in-depth hit validation using more labor-intensive approaches. The HEK293 cell line co-expressing CLuc-FGF14 and CD4-Nav1.6-NLuc was referred to as the LCA cell line.

Counter screening assays: Cell viability assay. The CellTiter-Blue® (CTB) Cell Viability Assay is a widely used, reliable fluorescent method for monitoring cell viability in HTS. The assay is based on the ability of living cells to convert a redox dye (resazurin) into a fluorescent end-product (resorufin). Nonviable cells rapidly lose metabolic capacity and thus do not generate a fluorescent signal. The assay is used in our pipeline as the first counter screening assay to eliminate toxic compounds. It is initiated by dispensing the proper amount of CTB reagent (i.e., 10 μL) per well in 384-well plates. Fluorescence is then read after 16 hrs, and cut-offs are set at a Z-score of <−3 to identify and exclude toxic compounds (FIG. 1B). The assay was typically run at a single, maximal concentration of the test compound calculated from the IC₅₀. Additional details about the use of this assay can be found in previous publications.

Full-length luciferase assay. To account for false positive results, compounds were tested against cells transfected with Photinus firefly luciferase control and selected on the basis of rigorous statistical criteria (FIG. 1C). The effect of compounds on the full-length luciferase assay was expressed as percent luminescence normalized to per-plate DMSO controls (n=16 per plate); compounds were selected based on (Z-score ≥3.11).

Dose responses. Non-toxic active compounds were tested using a 10-point dose response (n=4 per concentration) and selected based on estimated potency, efficacy, and structural diversity (IC₅₀). Blinded test-retest of compounds from the HTS library showed reliability with a R²≥0.75 (n=8 compounds, IC₅₀ value range across a 100-fold range). The HTS campaign provides evidence of throughput of >10 compounds per week, run with sufficient replicates (n=4) to produce robust and reproducible 10-point dose-response curves (FIG. 1D).

Secondary bioactivity assays: Surface plasmon resonance (SPR). Analogues were then tested using SPR against FGF14 and Nav1.6-C-terminal tail purified proteins, the gold standard for protein binding studies. SPR experiments were used to quantitatively assess the binding affinity of analogues, serving as both a secondary bioactivity assay to determine accurate binding affinities for protein:ligand interactions via K_{D}, Kₒₙ and K_{off}, as well as to assess the compound's ability to disrupt PPI. The reliability of the model, goodness of fit, and reported kinetic and binding constants were validated by varying flow rates, immobilization densities, or different chips. Statistical analysis was conducted by visually inspecting the goodness of fit of the data from the residuals. Additionally, c2 was used as a global measure for residual noise; standard deviation was used to report errors in measurements by repeating the experiment at least three times.

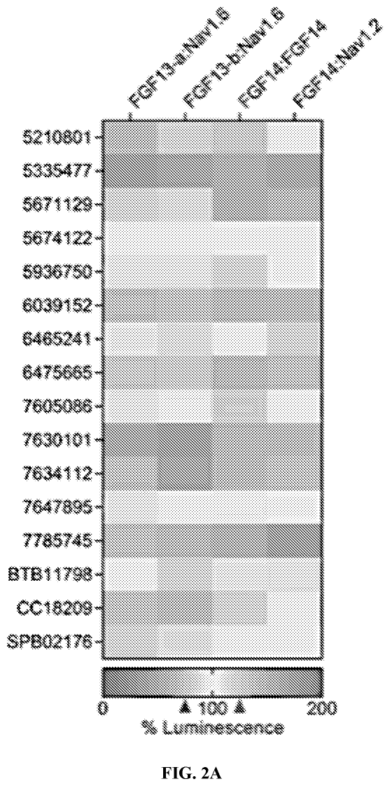

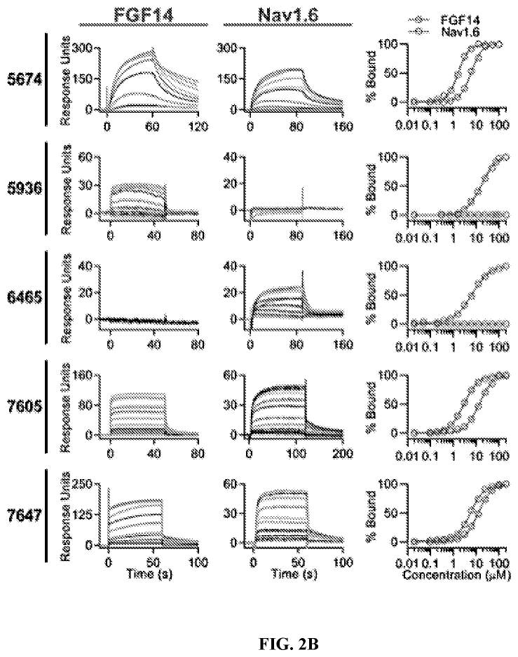

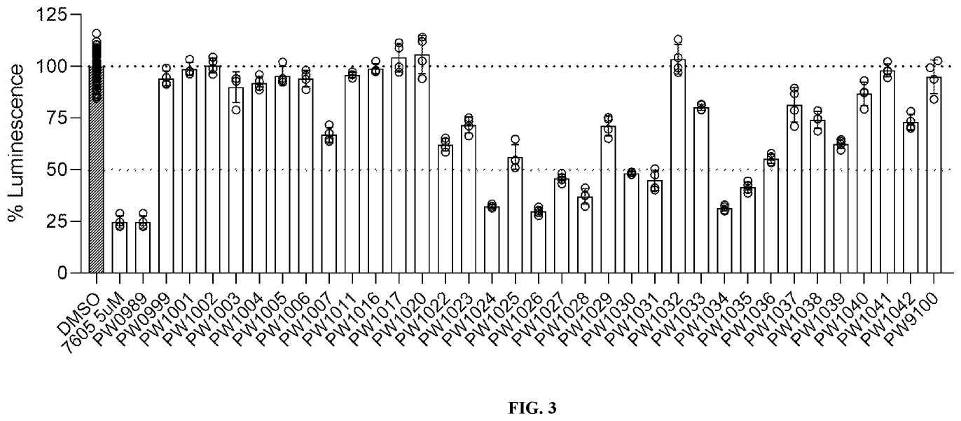

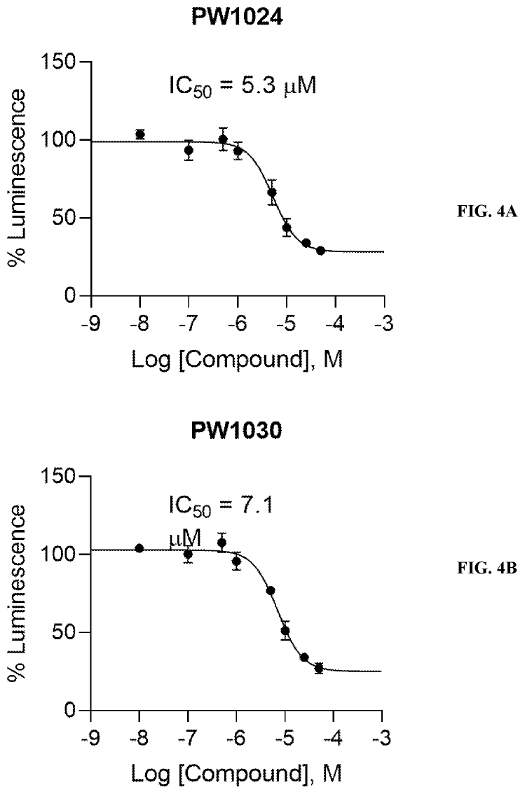

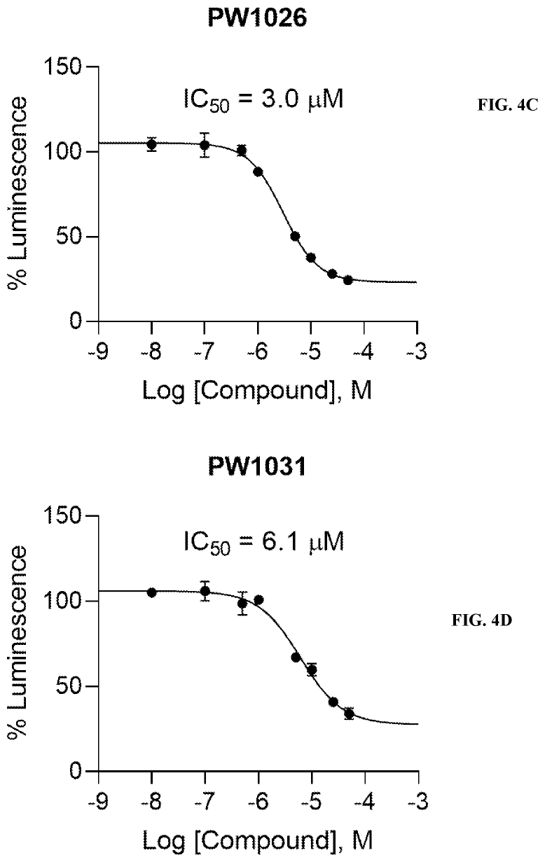

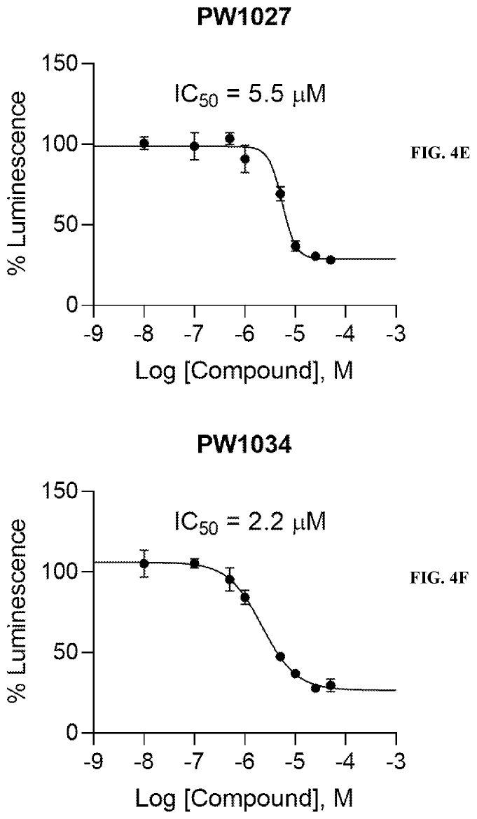

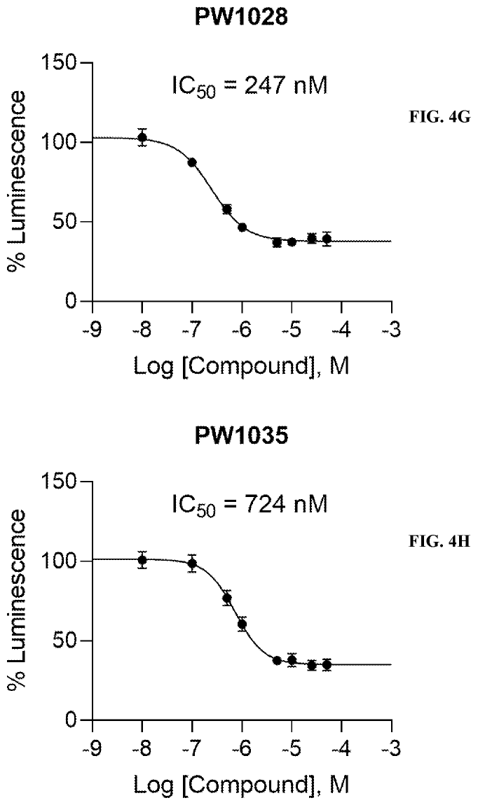

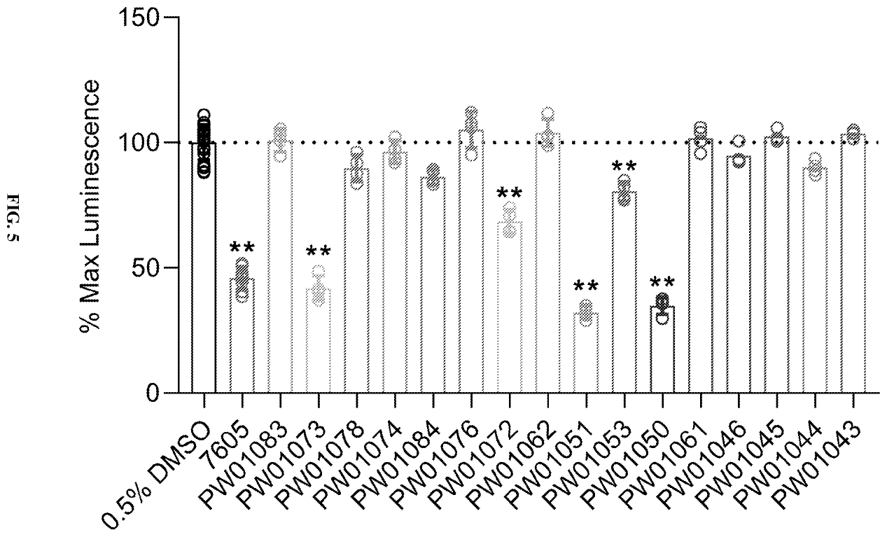

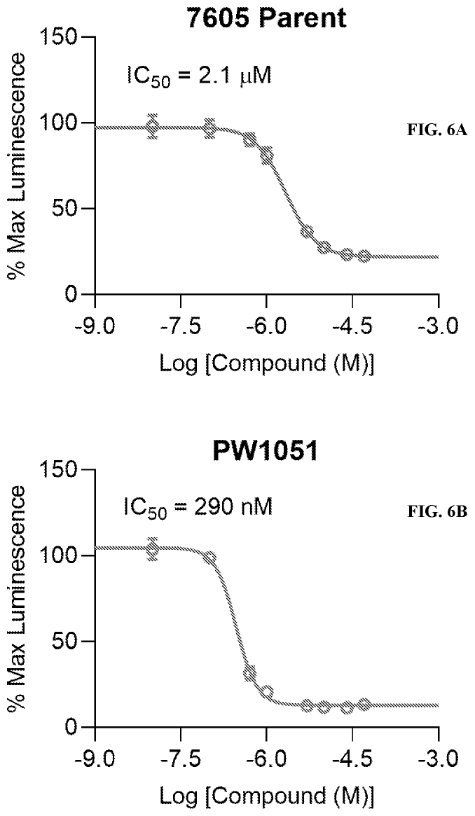

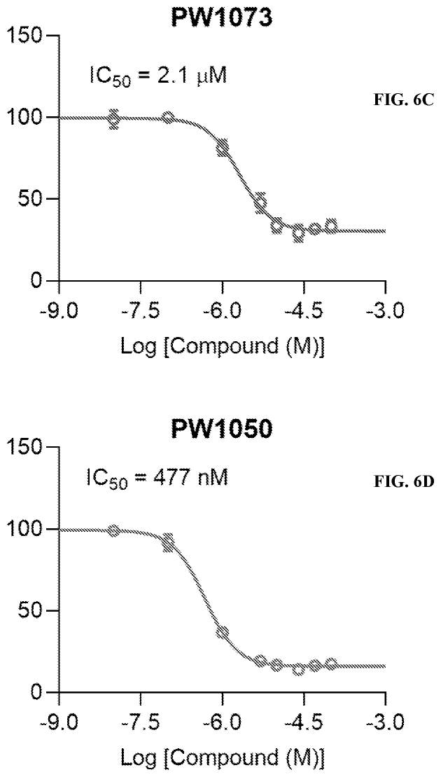

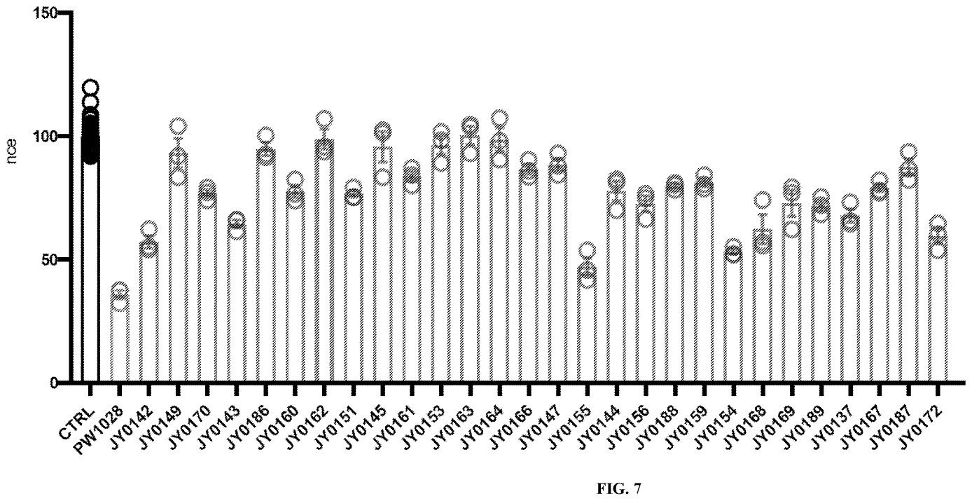

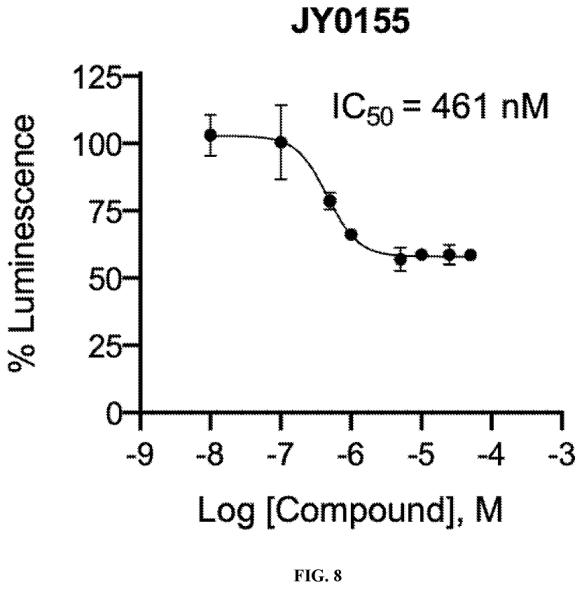

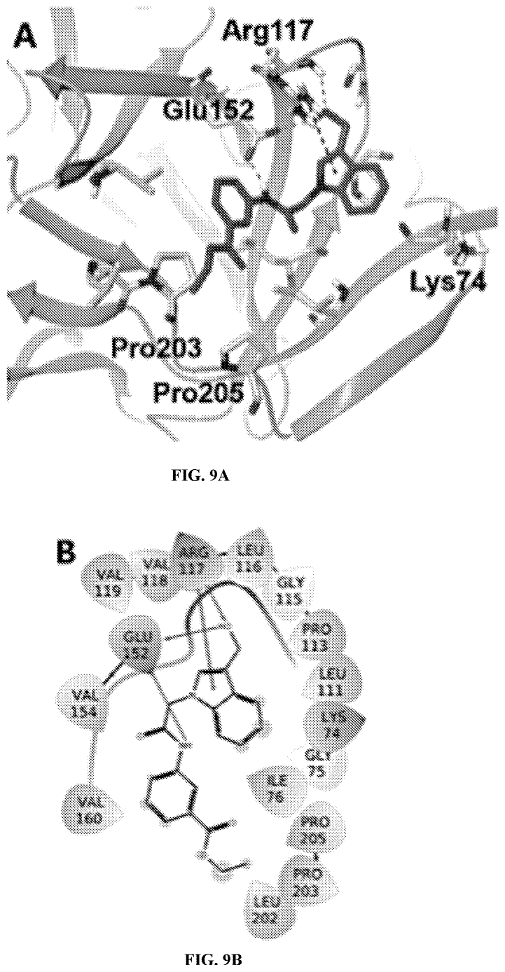

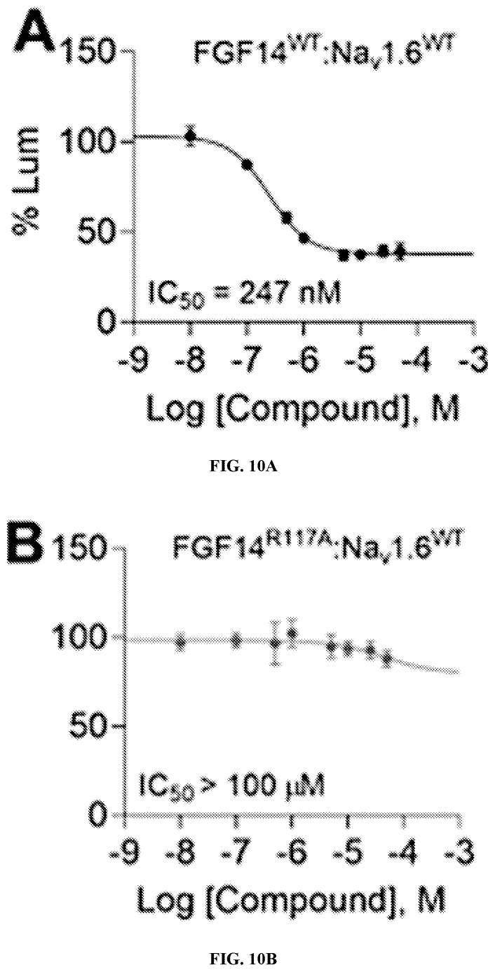

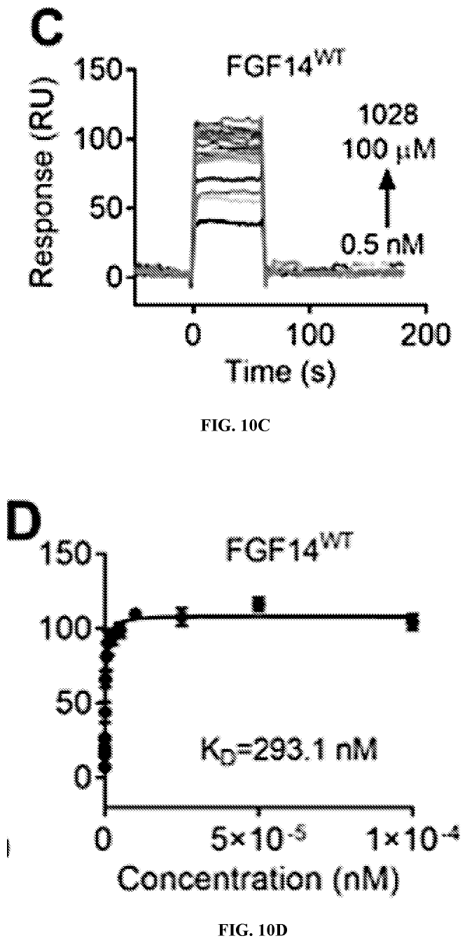

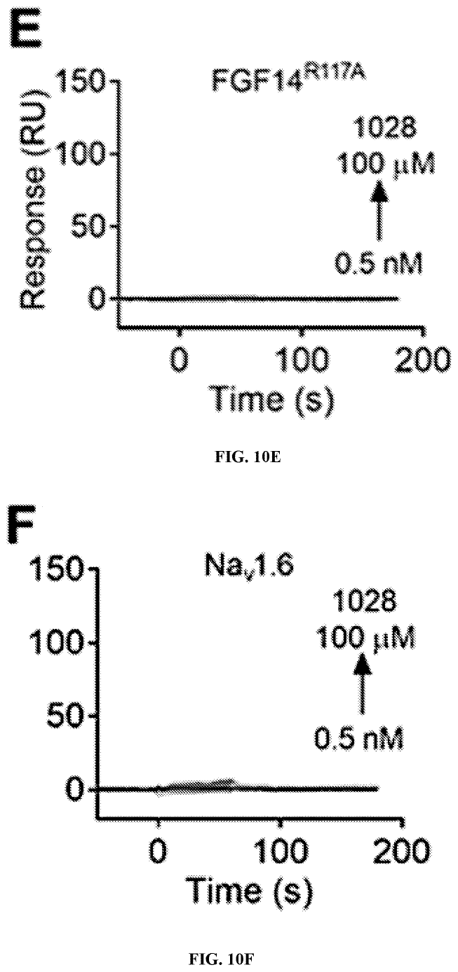

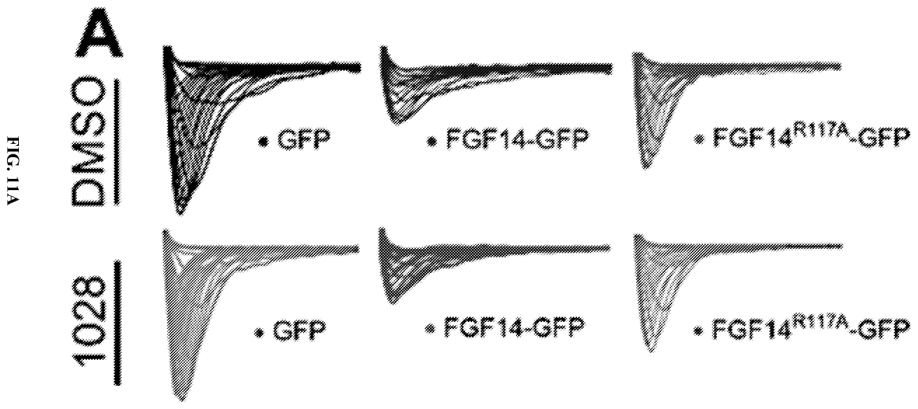

FIGS. 2A and 2B summarize selectivity and orthogonal validation of hits from the screening assay. As shown in FIG. 2A, the top 16 hits were screened (50 PM) against the FGF13-1a:Nav1.6, FGF13-1B:Nav1.6, and FGF14:Nav1.2 complexes, as well as the FGF14 dimer, using LCA. Compounds that modulated complex assembly by ≥25% were excluded from further studies. Referring to FIG. 2B, the remaining 5 selective hits were then quantitatively assessed using SPR. Binding sensorgrams are shown for FGF14 (left) and Nav1.6 (middle) and normalized steady-state saturation plots (right) reveal differences in compound:protein binding affinities. Kinetic analysis of each ligand/analyte interaction was obtained by fitting the response data to the simplest Langmuir 1:1 interaction model (K_{D}=k_{off}/kₒₙ). The K_{D} of compound 7605 was 2.9 mM for FGF14 and 13.6 mM for Nav1.6.

Confirmatory assays: Automated and manual patch-clamp electrophysiology in heterologous cells. Patch-clamp electrophysiology is a rigorous, sophisticated confirmatory and orthogonal assay that serves to assess compound functional activity, mechanism of action, specificity, and selectivity toward various Nav isoforms. It is highly predictive of in vivo efficacy of compounds. In this assay, compounds are tested in cells stably expressing FGF14 and Nav1.6 or other relevant iFGF:Nav channel pairs. Initially screened with an automated planar patch-clamp system, compounds were tested with an 8-point dose-response in 384-well plates with a throughput of >2,000 data sets/week. Compounds were then confirmed with manual patch-clamp at targeted concentrations. A multitude of physiological parameters of Nav channels including Na+ current amplitude, kinetics, voltage-dependence, use-dependency, and time-dependency reflecting the channel closed, opened, and inactivated states were automatically measured using specialized software (i.e., CLAMPEX). Data were typically derived from n=10-20 recordings, N=3 batches of cells. Data were compared using either Student's t-test or two-way ANOVA mixed model.

Patch-clamp electrophysiology in brain slices. This assay provides a critical confirmatory step from the heterologous cell to the native system. With this assay, selected compounds were tested (typically, 3 concentrations) for their modulatory effects on MSN firing and Na+ currents using current clamp and voltage clamp recordings Parameters of interest included action potential threshold, amplitude, rise time, firing frequency, amplitude of Na+ currents and passive properties (i.e., Rₛₑᵣᵢₑₛ, Rᵢₙₚᵤₜ, Vₘ). Not only is this assay predictive of a compound's in vivo activity, it provides an assessment of potential toxicity and side effects based on secondary measurements (i.e., frequency, amplitude, and kinetics of synaptic potentials, Rᵢₙₚᵤₜ, Vₘ). This assay was also used as the first target validation step in native tissue as compounds are concomitantly tested in neurons from Fgf14^{-/-} mice. Recordings were conducted from at least N=3 animals, n=10-15 recordings; effect is calculated using ANOVA mixed model.

Screening of compounds and selection of PW01028. Analogue potency was measured using a 10-point dose response in the CLuc-FGF14, CD4-Nav1.6 C-tail-NLuc double expressing cell line suitable for the LCA; counter screens against the full-length Photinus luciferase, along with the fluorescence-based cell viability test and selectivity against related targets were run in parallel using 4-point dose-response. FIGS. 2A and 2B summarize selectivity and orthogonal validation of hits from the screening assay.

As shown in FIG. 2A, the top 16 hits were screened (50 μM) against the FGF13-1a:Nav1.6, FGF13-1B:Nav1.6, and FGF14:Nav1.2 complexes, as well as the FGF14 dimer, using LCA. Compounds that modulated complex assembly by ≥25% were excluded from further studies.

Referring to FIG. 2B, the remaining 5 selective hits were then quantitatively assessed using SPR. Binding sensorgrams are shown for FGF14 (left) and Nav1.6 (middle) and normalized steady-state saturation plots (right) reveal differences in compound:protein binding affinities. Kinetic analysis of each ligand/analyte interaction was obtained by fitting the response data to the simplest Langmuir 1:1 interaction model (K_{D}=k_{off}/kₒₙ). The K_{D} of compound 7605086 was 2.9 mM for FGF14 and 13.6 mM for Nav1.6. Compound PW01028 displayed nanomolar potency in the LCA dose-response studies.

Functional validation and mechanism of action of optimized PW01028. FGF14 stands out among iFGFs as a potent, specific, and diverse modulator of Nav channels, especially Nav1.6. When expressed in recombinant cells, FGF14 suppressed or augmented Nav1.6-encoded currents and channel availability, depending on its N-terminal spliced isoform. In the native system, genetic deletion of FGF14 caused suppression of Nav1.6-mediated persistent and/or resurgent currents, while its overexpression increased Na⁺ current peak amplitude and channel availability. This suggested a complex functional role for FGF14 in modulating native Na⁺ currents that can be utilized to develop allosteric Nav1.6 modulators as probes for excitability.

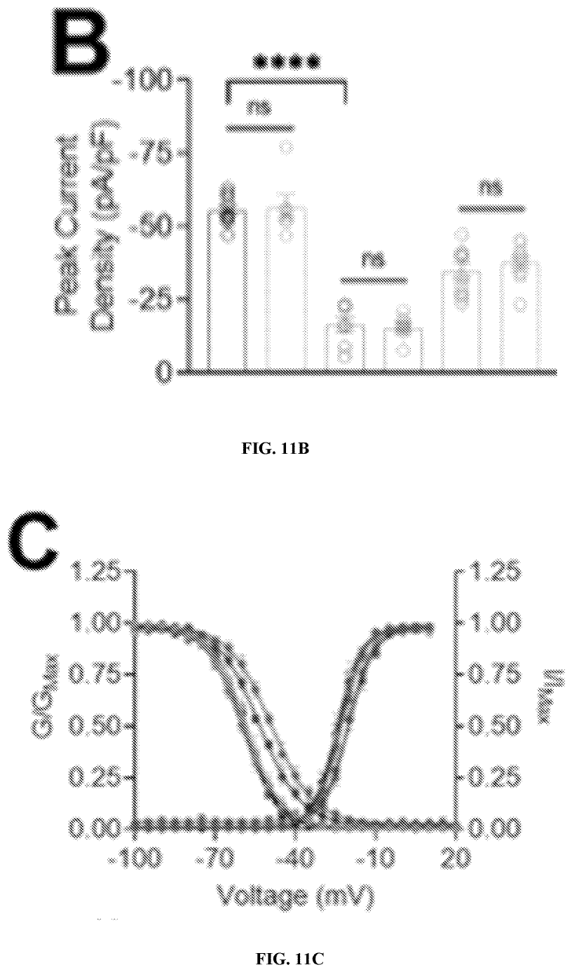

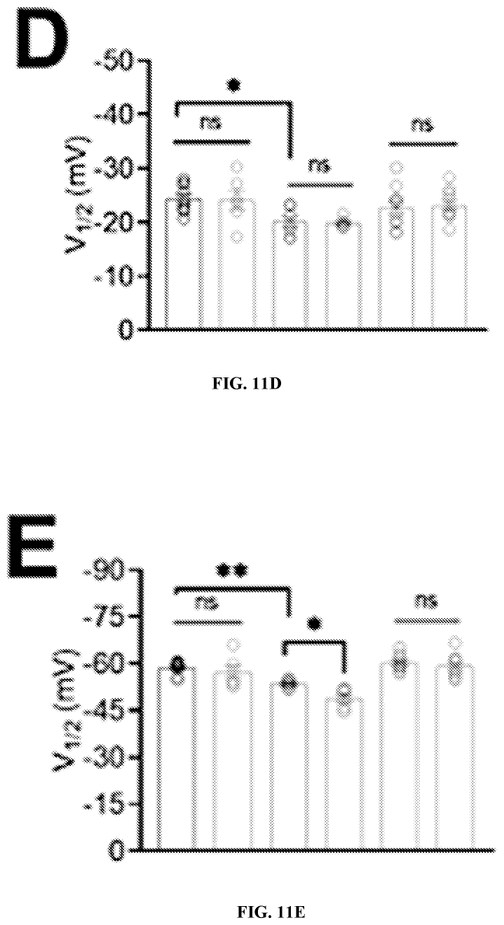

Functional activity of Compound PW01028. To study compound PW01028 functional activity against Nav1.6-encoded currents were studied in heterologous cells stably expressing Nav1.6 and/or FGF14-GFP (FIGURE). From these studies it was concluded that compound PW01028 markedly shifted V_{1/2} and steady-state inactivation to a more depolarized potential. If reproduced in MSN, this phenotype is expected to increase intrinsic firing. Notably, compound PW01028 had no functional effects in HEK-Nav1.6 expressing the GFP control construct which provides evidence of target validation of compound PW01028 (FIG. 11). Importantly, these results demonstrate that probes targeting the FGF14:Nav1.6 PPI interface act on non-conserved allosteric surfaces within the Nav channel complex, and as such, may exhibit a higher degree of specificity compared to any other known Nav channel modulators.

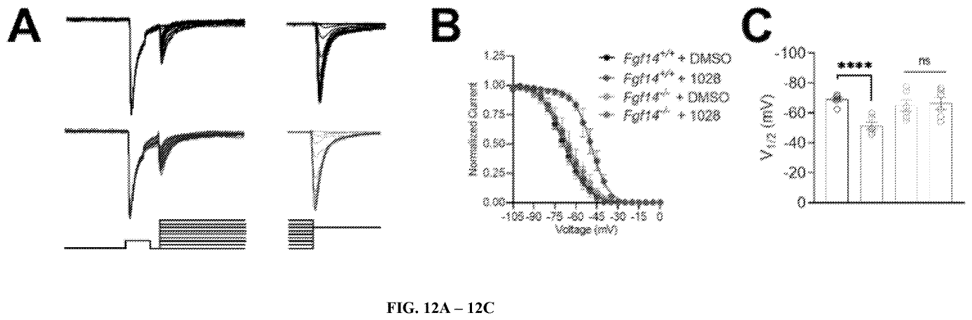

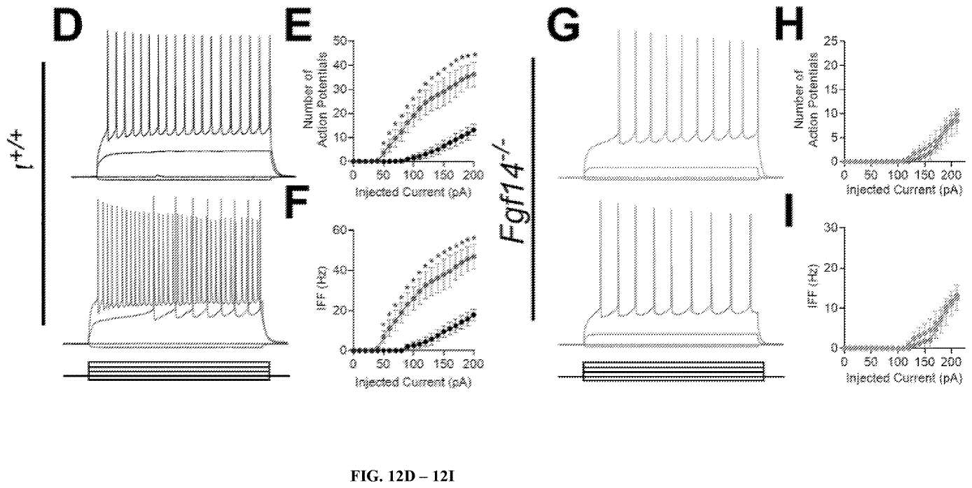

Target validation of compound PW01028 in NAc slices. As expected from heterologous cell studies (FIG. 12), treatment with compound PW01028 (1 micromolar) potentiated MSN firing and transient Na⁺ currents (I_{NaP}) (FIG. 12) in Fgf14^{+/+} mice but was inactive in MSN from Fgf14^{-/-} mice (FIG. 12G-I), providing evidence for target validation of compound PW01028.

### 4.1. Chemistry

All commercially available starting materials and solvents were reagent grade and used without further purification. Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). Visualization of the developed chromatograms was performed with detection by UV (254 nm). NMR spectra were recorded on a Brucker-300 (¹H, 300 MHz; ¹³C, 75 MHz; ¹⁹F, 282 MHz) spectrometer. ¹H and ¹³C NMR spectra were recorded with TMS as an internal reference. Chemical shifts were expressed in ppm, and J values were given in Hz. High-resolution mass spectra (HRMS) were obtained from Thermo Fisher LTQ Orbitrap Elite mass spectrometer. Parameters include the following: Nano ESI spray voltage was 1.8 kV; Capillary temperature was 275° C. and the resolution was 60,000; Ionization was achieved by positive mode. Melting points were measured on a Thermo Scientific Electrothermal Digital Melting Point Apparatus and uncorrected. Purities of final compounds were established by analytical HPLC, which was carried out on a Shimadzu HPLC system (model: CBM-20A LC-20AD SPD-20A UV/VIS). HPLC analysis conditions: Waters μBondapak C18 (300×3.9 mm); flow rate 0.5 mL/min; UV detection at 270 and 254 nm; linear gradient from 10% acetonitrile in water to 100% acetonitrile in water in 20 min followed by 30 min of the last-named solvent (0.1% TFA was added into both acetonitrile and water). All biologically evaluated compounds were >95% pure.

### Synthetic Experimental Procedures for Synthesis of PW01028 Analogs

### Synthesis of 7605086 (Also Known as PW0989)

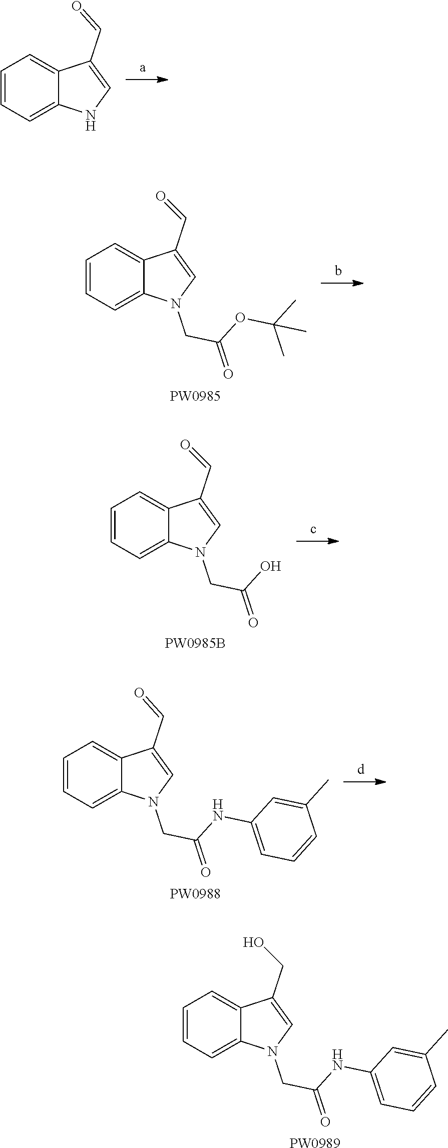

Reagents and conditions: (a) tert-butyl 2-bromoacetate, K₂CO₃, DMF, rt, overnight, 96%. (b) TFA, rt, 4 h, quant. (c) m-toluidine, EDCI, DMAP, DMF, rt, overnight, 82%. (d) NaBH₄, MeOH, 0° C., 1 h, 79%.

tert-Butyl 2-(3-formyl-1H-indol-1-yl)acetate (PW0985). A solution of tert-butyl 2-bromoacetate (4.7 g, 24 mmol) in DMF (20 mL) was slowly added to a stirred mixture of 1H-indole-3-carbaldehyde (2.9 g, 20 mmol) and K₂CO₃ (5.5 g, 40 mmol) in 20 mL of dry DMF. The reaction solution was stirred at rt overnight until TLC indicated the end of reaction. Then poured the solution into ice water (200 mL) and then extracted with EtOAc (100 mL×3). The organic phase was washed with brine, dried over Na₂SO₄, and then concentrated under reduced pressure. The residue was purified by flash column chromatography (DCM/MeOH=100/1) to get the PW0985 as a white solid (5.0 g, 96%). ¹H NMR (300 MHz, Chloroform-d) δ 10.02 (s, 1H), 8.38-8.27 (m, 1H), 7.74 (s, 1H), 7.31 (dddd, J=16.8, 6.1, 3.5, 1.7 Hz, 3H), 4.80 (s, 2H), 1.46 (s, 9H).

2-(3-Formyl-1H-indol-1-yl)acetic acid. In a round-bottom flask, compound (2.6 g, 10 mmol) was dissolved in TFA (15 mL). The reaction solution was stirred at rt for 2 h until TLC indicated the end of reaction. After concentrated under reduced pressure to remove of the solvent, the residue was freeze-dried on a lyophilizer to get a white solid, which was used directly to the next step without further purification. ¹H NMR (300 MHz, DMSO-d₆) δ 9.93 (s, 1H), 8.28 (s, 1H), 8.16-8.06 (m, 1H), 7.58-7.52 (m, 1H), 7.37-7.20 (m, 2H), 5.18 (s, 2H).

2-(3-Formyl-1H-indol-1-yl)-N-(m-tolyl)acetamide (PW0988). In a round-bottom flask, compound (1.8 g, 7 mmol) was dissolved in 25 mL of anhydrous DMF, and the solution was cooled to 0° C. with an ice bath. m-Toluidine (1.1 g, 10.5 mmol), EDCI (2.7 g, 14 mmol), DMAP (1.7 g, 14 mmol) were added to the solution at 0° C. Then removed the ice bath and the mixture solution was stirred at rt overnight until TLC indicated the end of reaction. Then poured the solution into ice water (100 mL) and extracted with EtOAc (50 mL×3). The organic phase was washed with brine, dried over Na₂SO₄, and then concentrated under reduced pressure. The residue was purified by flash column chromatography (DCM/MeOH=50/1) to get the PW0988 as a white solid (1.4 g, 82%). ¹H NMR (300 MHz, DMSO-d₆) δ 10.43 (s, 1H), 9.96 (s, 1H), 8.34 (s, 1H), 8.13 (dd, J=7.8, 1.4 Hz, 1H), 7.59-7.53 (m, 1H), 7.44 (d, J=2.0 Hz, 1H), 7.38 (d, J=8.4 Hz, 1H), 7.30 (dtd, J=13.8, 7.1, 1.3 Hz, 2H), 7.21 (t, J=7.8 Hz, 1H), 6.90 (d, J=7.5 Hz, 1H), 5.21 (s, 2H), 2.27 (s, 3H).

2-(3-(Hydroxymethyl)-1H-indol-1-yl)-N-(m-tolyl)acetamide (PW0989). In a round-bottom flask, compound (1.5 g, 5 mmol) was dissolved in 20 mL of MeOH, and the solution was cooled to 0° C. with an ice bath. NaBH₄ (190 mg, 5 mmol) was slowly added to solution in a small portion and the solution was continually stirred for 1 h until TLC indicated the end of reaction. Then poured the solution into ice water (100 mL) and extracted with EtOAc (50 mL×3). The organic phase was washed with brine, dried over Na₂SO₄, and then concentrated under reduced pressure. The residue was purified by flash column chromatography (DCM/MeOH=50/1) to get the PW0989 as a white solid (1.2 g, 82%). ¹H NMR (300 MHz, DMSO-d₆) δ 10.27 (s, 1H), 7.62 (dt, J=7.7, 1.0 Hz, 1H), 7.47-7.34 (m, 3H), 7.29 (s, 1H), 7.24-7.09 (m, 2H), 7.04 (ddd, J=7.9, 7.0, 1.1 Hz, 1H), 6.89 (d, J=7.5 Hz, 1H), 4.99 (s, 2H), 4.82 (t, J=5.4 Hz, 1H), 4.72-4.60 (m, 2H), 2.27 (s, 3H).

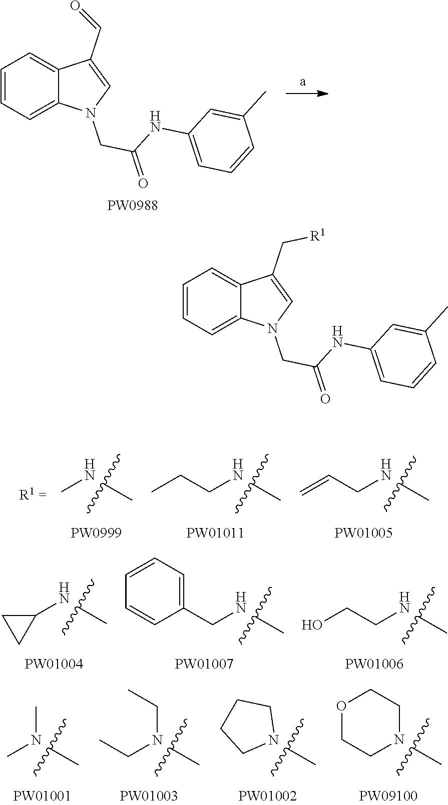

Reagents and conditions: (a) corresponding amine, NaBH(OAc)₃, 1,2-dichloroethane, 0° C.~rt, 34%-82%.

2-(3-((Methylamino)methyl)-1H-indol-1-yl)-N-(m-tolyl)acetamide (PW0999). A RB flask with magnetic stir bar was charged with 2 mL of 1,2-dichloroethane, compound PW0988(58.4 mg, 0.2 mmol), and CH₃NH₂ (27 mg, 0.4 mmol). The solution was cooled to 0° C. with an ice bath. To the stirred reaction solution under a nitrogen atmosphere was added NaBH(OAc)₃ (85 mg, 0.4 mmol) and AcOH (12 mg, 0.2 mmol). Then, removed the ice bath and the flask was sealed under nitrogen and stirred at room temperature overnight. The reaction was poured in water, and then extracted with CH₂Cl₂ (10 mL×3). The organic phase was washed with brine, dried over Na₂SO₄, and then concentrated under reduced pressure. The residue was purified by flash column chromatography with DCM/MeOH to get the PW0999 as a colorless oil (21 mg, 34%). ¹H NMR (300 MHz, Chloroform-d) δ 7.68 (dd, J=7.6, 1.3 Hz, 1H), 7.56 (s, 1H), 7.35-7.28 (m, 2H), 7.27-7.21 (m, 1H), 7.20-7.10 (m, 3H), 7.08 (s, 1H), 6.90 (d, J=7.0 Hz, 1H), 4.89 (s, 2H), 3.94 (s, 2H), 2.74 (s, 1H), 2.43 (s, 3H), 2.26 (s, 3H). ¹³C NMR (75 MHz, CDCl₃) δ 166.6, 166.6, 138.9, 136.6, 128.7, 127.7, 127.3, 125.8, 123.2, 120.9, 120.6, 119.5, 117.4, 109.5, 50.3, 46.0, 35.6, 21.3. HRMS (ESI) calcd for C₁₉H₂₂N₃O, 308.1757 (M+H)⁺, found 308.1762.

2-(3-((Propylamino)methyl)-1H-indol-1-yl)-N-(m-tolyl)acetamide (PW01011). Following the similar synthetic procedure that used to prepare compound PW0999, compound PW01011 as a colorless oil (46 mg, 69%). ¹H NMR (300 MHz, Chloroform-d) δ 7.72 (d, J=7.8 Hz, 1H), 7.31 (d, J=6.6 Hz, 2H), 7.23 (td, J=8.2, 4.0 Hz, 2H), 7.13 (d, J=3.5 Hz, 4H), 6.91 (d, J=6.6 Hz, 1H), 4.87 (s, 2H), 4.03 (s, 2H), 2.73 (t, J=7.3 Hz, 2H), 2.28 (s, 3H), 1.59 (q, J=7.3 Hz, 2H), 0.96 (t, J=7.4 Hz, 3H). ¹³C NMR (75 MHz, CDCl₃) δ 166.5, 138.9, 136.8, 136.5, 128.7, 128.0, 126.5, 125.8, 123.2, 120.9, 120.5, 119.5, 117.4, 109.4, 53.4, 51.6, 50.5, 44.4, 23.0, 21.3, 11.8. HRMS (ESI) calcd for C₂₁H₂₅N₃O, 336.2070 (M+H)⁺, found 336.2077.

2-(3-((Allylamino)methyl)-1H-indol-1-yl)-N-(m-tolyl)acetamide (PW01005). Following the similar synthetic procedure that used to prepare compound PW0999, compound PW01005 as a colorless oil (37 mg, 56%). ¹H NMR (300 MHz, Chloroform-d) δ 7.74 (d, J=7.8 Hz, 1H), 7.31 (dd, J=5.9, 1.4 Hz, 2H), 7.24 (dt, J=4.0, 2.1 Hz, 2H), 7.17-7.10 (m, 4H), 6.92 (dt, J=8.4, 4.3 Hz, 1H), 5.99 (ddt, J=16.4, 10.2, 6.0 Hz, 1H), 5.30-5.13 (m, 2H), 4.85 (s, 2H), 4.03 (s, 2H), 3.39 (dt, J=5.9, 1.5 Hz, 2H), 2.28 (s, 3H). ¹³C NMR (75 MHz, CDCl₃) δ 166.5, 138.9, 136.8, 136.6, 136.5, 128.8, 128.0, 126.5, 125.8, 123.2, 121.0, 120.5, 119.6, 117.5, 116.3, 109.4, 52.1, 50.4, 43.9, 21.3. HRMS (ESI) calcd for C₂₁H₂₄N₃O, 334.1914 (M+H)⁺, found 334.1922.

2-(3-((Cyclopropylamino)methyl)-1H-indol-1-yl)-N-(m-tolyl)acetamide (PW01004). Following the similar synthetic procedure that used to prepare compound PW0999, compound PW01004 as a colorless oil (46 mg, 69%). ¹H NMR (300 MHz, Chloroform-d) δ 7.83-7.69 (m, 1H), 7.37-7.28 (m, 2H), 7.24 (ddd, J=8.0, 5.7, 2.4 Hz, 1H), 7.20-7.04 (m, 5H), 6.93 (dd, J=6.9, 2.1 Hz, 1H), 4.85 (s, 2H), 4.10 (s, 2H), 2.29 (s, 4H), 0.50 (ddt, J=8.2, 4.0, 2.0 Hz, 4H). ¹³C NMR (75 MHz, CDCl₃) δ 166.4, 138.9, 136.8, 136.5, 128.8, 128.2, 126.3, 125.8, 123.2, 120.9, 120.4, 119.7, 117.4, 117.0, 109.3, 50.5, 44.3, 30.5, 21.4, 6.5. HRMS (ESI) calcd for C₂₁H₂₄N₃O, 334.1914 (M+H)⁺, found 334.1922.

2-(3-((Benzylamino)methyl)-1H-indol-1-yl)-N-(m-tolyl)acetamide (PW01007). Following the similar synthetic procedure that used to prepare compound PW0999, compound PW01007 as a colorless oil (60 mg, 79%). ¹H NMR (300 MHz, Chloroform-d) δ 7.74 (dd, J=7.7, 1.3 Hz, 1H), 7.46-7.28 (m, 7H), 7.27-7.18 (m, 2H), 7.18-7.12 (m, 3H), 7.10 (s, 1H), 7.00-6.87 (m, 1H), 4.85 (s, 2H), 4.06 (s, 2H), 3.94 (s, 2H), 2.29 (s, 3H). ¹³C NMR (75 MHz, CDCl₃) δ 166.5, 140.2, 138.9, 136.9, 136.5, 128.8, 128.5, 128.2, 128.1, 127.1, 126.5, 125.8, 123.2, 120.9, 120.5, 119.7, 117.5, 116.5, 109.3, 53.6, 50.5, 44.1, 21.3. HRMS (ESI) calcd for C₂₅H₂₆N₃O, 384.2070 (M+H)⁺, found 384.2079.

2-(3-(((2-Hydroxyethyl)amino)methyl)-1H-indol-1-yl)-N-(m-tolyl)acetamide (PW01006). Following the similar synthetic procedure that used to prepare compound PW0999, compound PW01006 as a white solid (49 mg, 73%). ¹H NMR (300 MHz, DMSO-d₆) δ 10.24 (s, 1H), 7.63 (d, J=7.8 Hz, 1H), 7.49-7.32 (m, 3H), 7.28 (s, 1H), 7.16 (dt, J=19.1, 7.7 Hz, 2H), 7.03 (t, J=7.4 Hz, 1H), 6.88 (d, J=7.5 Hz, 1H), 4.99 (s, 2H), 4.48 (s, 1H), 3.90 (s, 2H), 3.57-3.44 (m, 2H), 2.68 (t, J=5.7 Hz, 2H), 2.26 (s, 3H). ¹³C NMR (75 MHz, DMSO) δ 166.8, 139.1, 138.5, 137.3, 129.1, 128.6, 127.9, 124.6, 121.7, 120.2, 119.5, 119.1, 116.8, 113.6, 110.1, 60.6, 51.6, 49.6, 44.5, 21.6. HRMS (ESI) calcd for C₂₀H₂₄N₃O₂ 338.1863 (M+H)⁺, found 338.1870.

2-(3-((Dimethylamino)methyl)-1H-indol-1-yl)-N-(m-tolyl)acetamide (PW01001). Following the similar synthetic procedure that used to prepare compound PW0999, compound PW01001 as a white solid (51 mg, 82%). ¹H NMR (300 MHz, Chloroform-d) δ 7.80-7.71 (m, 1H), 7.43-7.26 (m, 3H), 7.22 (ddd, J=8.0, 6.7, 1.5 Hz, 1H), 7.19-7.05 (m, 4H), 6.96-6.86 (m, 1H), 4.88 (s, 2H), 3.72 (s, 2H), 2.36 (s, 6H), 2.27 (s, 3H). ¹³C NMR (75 MHz, CDCl₃) δ 166.4, 138.9, 136.6, 128.8, 128.6, 128.1, 125.8, 123.1, 120.9, 120.6, 119.8, 117.4, 113.3, 109.4, 54.1, 50.4, 45.0, 21.3. HRMS (ESI) calcd for C₂₀H₂₄N₃O, 322.1914 (M+H)⁺, found 322.1921.

2-(3-((Diethylamino)methyl)-1H-indol-1-yl)-N-(m-tolyl)acetamide (PW01003). Following the similar synthetic procedure that used to prepare compound PW0999, compound PW01003 as a white solid (41 mg, 59%). ¹H NMR (300 MHz, Chloroform-d) δ 7.84 (d, J=7.8 Hz, 1H), 7.40-7.28 (m, 2H), 7.28-7.14 (m, 2H), 7.12 (dd, J=4.5, 2.9 Hz, 3H), 7.00 (s, 1H), 6.92 (d, J=7.0 Hz, 1H), 4.89 (s, 2H), 3.84 (s, 2H), 2.63 (q, J=7.1 Hz, 4H), 2.29 (s, 3H), 1.14 (t, J=7.1 Hz, 6H). ¹³C NMR (75 MHz, CDCl₃) δ 166.5, 138.9, 136.7, 136.5, 129.0, 128.8, 127.0, 125.8, 123.1, 120.7, 120.4, 117.2, 115.7, 109.1, 50.5, 48.1, 46.9, 21.3, 12.0. HRMS (ESI) calcd for C₂₂H₂₈N₃O, 350.2227 (M+H)⁺, found 350.2234.

2-(3-(Pyrrolidin-1-ylmethyl)-1H-indol-1-yl)-N-(m-tolyl)acetamide (PW01002). Following the similar synthetic procedure that used to prepare compound PW0999, compound PW01002 as a white solid (53 mg, 74%). ¹H NMR (300 MHz, Chloroform-d) δ 7.87-7.61 (m, 2H), 7.37-7.30 (m, 1H), 7.30-7.09 (m, 6H), 6.90 (d, J=7.2 Hz, 1H), 4.84 (s, 2H), 3.93 (s, 2H), 2.88-2.70 (m, 4H), 2.26 (s, 3H), 1.83 (q, J=3.4 Hz, 4H). ¹³C NMR (75 MHz, CDCl₃) δ 166.4, 138.9, 136.8, 136.6, 128.8, 128.7, 128.2, 125.7, 123.1, 120.9, 120.7, 119.4, 117.4, 111.4, 109.6, 53.8, 50.3, 49.8, 23.3, 21.3. HRMS (ESI) calcd for C₂₂H₂₆N₃O, 348.2070 (M+H)⁺, found 348.2079.

2-(3-(Morpholinomethyl)-1H-indol-1-yl)-N-(m-tolyl)acetamide (PW09100). Following the similar synthetic procedure that used to prepare compound PW0999, compound PW09100 as a white solid (57 mg, 79%). ¹H NMR (300 MHz, Chloroform-d) δ 7.84 (d, J=7.8 Hz, 1H), 7.38-7.29 (m, 2H), 7.27-7.19 (m, 2H), 7.14 (dd, J=7.4, 5.0 Hz, 4H), 6.93 (dd, J=6.7, 2.1 Hz, 1H), 4.88 (s, 2H), 3.81-3.70 (m, 6H), 2.56 (dd, J=5.7, 3.6 Hz, 4H), 2.29 (s, 3H). ¹³C NMR (75 MHz, CDCl₃) δ 166.4, 139.0, 136.8, 136.5, 128.8, 127.6, 125.9, 123.2, 120.9, 120.5, 120.3, 117.4, 113.5, 109.3, 67.0, 53.8, 53.6, 50.4, 21.4. HRMS (ESI) calcd for C₂₂H₂₆N₃O₂ 364.2020 (M+H)⁺, found 364.2029.

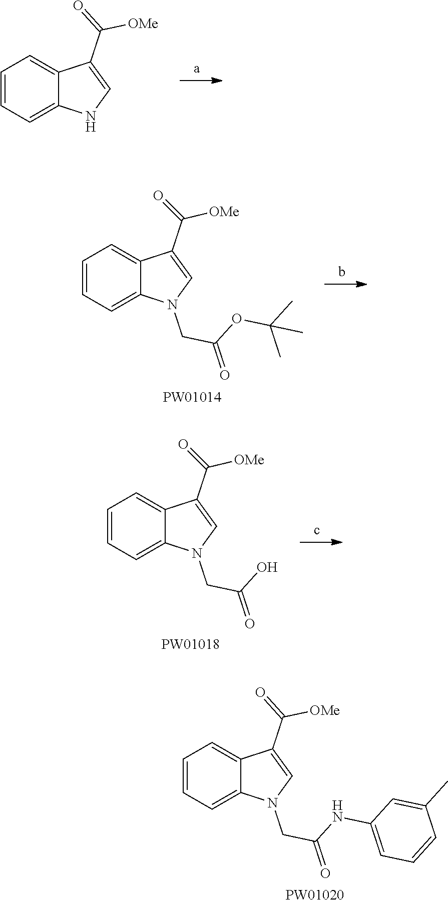

Reagents and conditions: (a) tert-butyl 2-bromoacetate, K₂CO₃, DMF, rt, overnight, 93%. (b) TFA, rt, 4 h, quant. (c) (1) m-toluidine, EDCI, DMAP, DMF, rt, overnight, 86%.

Methyl 1-(2-(tert-butoxy)-2-oxoethyl)-1H-indole-3-carboxylate (PW01014). Following the similar synthetic procedure that used to prepare compound PW0985, compound PW01014 as a white solid (540 mg, 93%). ¹H NMR (300 MHz, Chloroform-d) δ 8.25-8.17 (m, 1H), 7.84 (s, 1H), 7.35-7.28 (m, 3H), 4.78 (s, 2H), 3.93 (s, 3H), 1.46 (s, 9H).

2-(3-(Methoxycarbonyl)-1H-indol-1-yl)acetic acid (PW01018). Following the similar synthetic procedure that used to prepare compound PW0985, compound PW01018 as a white solid (quant.). ¹H NMR (300 MHz, DMSO-d₆) δ 13.15 (s, 1H), 8.14 (s, 1H), 8.06-7.95 (m, 1H), 7.55-7.47 (m, 1H), 7.30-7.18 (m, 2H), 5.14 (s, 2H), 3.82 (s, 3H).

Methyl 1-(2-oxo-2-(m-tolylamino)ethyl)-1H-indole-3-carboxylate (PW01020). Following the similar synthetic procedure that used to prepare compound PW0985, compound PW01020 as a white solid (55 mg, 89%). ¹H NMR (300 MHz, DMSO-d₆) δ 10.36 (s, 1H), 8.18 (s, 1H), 8.08-7.99 (m, 1H), 7.54-7.48 (m, 1H), 7.43 (t, J=1.8 Hz, 1H), 7.37 (d, J=8.3 Hz, 1H), 7.30-7.16 (m, 3H), 6.89 (d, J=7.5 Hz, 1H), 5.16 (s, 2H), 3.83 (s, 3H), 2.27 (s, 3H). ¹³C NMR (75 MHz, DMSO) δ 165.8, 164.9, 138.9, 138.5, 137.4, 137.3, 129.1, 126.5, 124.8, 123.0, 122.1, 121.1, 120.2, 116.8, 111.2, 106.2, 51.2, 49.9, 21.6. HRMS (ESI) calcd for C₁₉H₁₉N₂O₃ 323.1390 (M+H)⁺, found 323.1398.

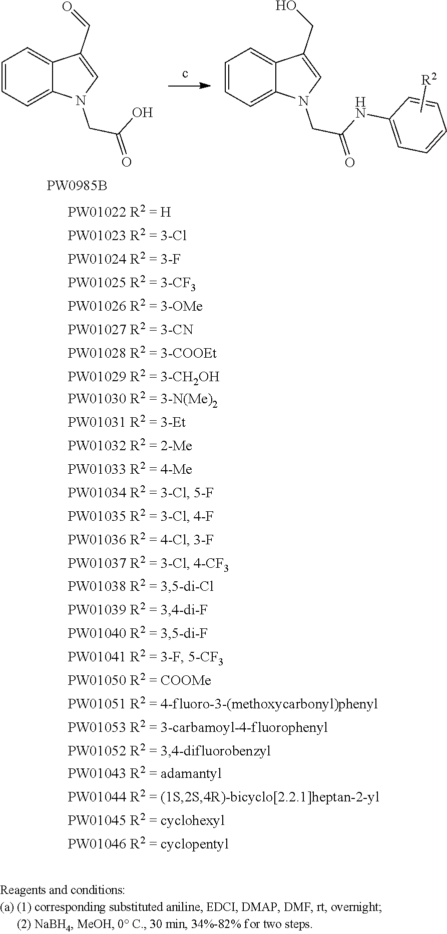

2-(3-(Hydroxymethyl)-1H-indol-1-yl)-N-phenylacetamide (PW01022). In a round-bottom flask, compound PW0985B (52 mg, 0.2 mmol) was dissolved in 2 mL of anhydrous DMF, and the solution was cooled to 0° C. with an ice bath. aniline (37 mg, 0.4 mmol), EDCI (58 mg, 0.3 mmol), DMAP (49 mg, 0.4 mmol) were added to the solution at 0° C. Then removed the ice bath and the mixture solution was stirred at rt overnight until TLC indicated the end of reaction. Then poured the solution into ice water (10 mL) and extracted with EtOAc (10 mL×3). The organic phase was washed with 1N HCl, 1N NaHCO₃, and brine, dried over Na₂SO₄, and then concentrated under reduced pressure. The residue was dissolved in 5 mL of MeOH, and the solution was cooled to 0° C. with an ice bath. NaBH₄ (15 mg, 0.4 mmol) was added to solution and the solution was continually stirred for 30 min until TLC indicated the end of reaction. Then poured the solution into ice water (10 mL) and extracted with EtOAc (10 mL×3). The organic phase was washed with brine, dried over Na₂SO₄, and then concentrated under reduced pressure. The residue was purified by flash column chromatography (DCM/MeOH=30/1) to get the PW01022 as a white solid (48 mg, 86%). ¹H NMR (300 MHz, Methanol-d₄) δ 7.71 (dt, J=7.8, 1.0 Hz, 1H), 7.56-7.51 (m, 2H), 7.39-7.26 (m, 4H), 7.24-7.17 (m, 1H), 7.15-7.08 (m, 2H), 4.99 (s, 2H), 4.83 (s, 2H). ¹³C NMR (75 MHz, MeOD) δ 167.5, 137.9, 137.4, 128.4, 127.7, 127.6, 124.1, 121.7, 120.0, 119.2, 118.8, 115.5, 109.0, 55.6, 49.0. HRMS (ESI) calcd for C₁₇H₁₇N₂O₂ 281.1285 (M+H)⁺, found 281.1285.

N-(3-Chlorophenyl)-2-(3-(hydroxymethyl)-1H-indol-1-yl)acetamide (PW01023). Following the similar synthetic procedure that used to prepare compound PW01022, compound PW01023 as a white solid (47 mg, 76%). ¹H NMR (300 MHz, DMSO-d₆) δ 10.59 (s, 1H), 7.79 (s, 1H), 7.61 (d, J=7.7 Hz, 1H), 7.48 (d, J=8.1 Hz, 1H), 7.41-7.31 (m, 2H), 7.28 (s, 1H), 7.12 (d, J=7.7 Hz, 2H), 7.03 (t, J=7.4 Hz, 1H), 5.03 (s, 2H), 4.80 (t, J=5.4 Hz, 1H), 4.65 (d, J=5.3 Hz, 2H). ¹³C NMR (75 MHz, DMSO) δ 167.3, 140.6, 137.3, 133.6, 131.0, 128.2, 127.5, 123.7, 121.8, 119.6, 119.2, 119.1, 118.0, 116.3, 110.1, 55.7, 49.5. HRMS (ESI) calcd for C₁₇H₁₆ClN₂O₂Na, 337.0714 (M+Na)⁺, found 337.0708.

N-(3-Fluorophenyl)-2-(3-(hydroxymethyl)-1H-indol-1-yl)acetamide (PW01024). Following the similar synthetic procedure that used to prepare compound PW01022, compound PW01024 as a white solid (44 mg, 73%). ¹H NMR (300 MHz, DMSO-d₆) δ 10.56 (s, 1H), 7.69-7.50 (m, 2H), 7.44-7.25 (m, 4H), 7.13 (ddd, J=8.2, 7.0, 1.3 Hz, 1H), 7.03 (ddd, J=8.0, 7.0, 1.1 Hz, 1H), 6.95-6.83 (m, 1H), 5.02 (s, 2H), 4.81 (t, J=5.4 Hz, 1H), 4.71-4.56 (m, 2H). ¹³C NMR (75 MHz, DMSO) δ 167.3, 162.6 (d, J=241.5 Hz), 140.9 (d, J=11.0 Hz), 137.4, 131.0 (d, J=9.5 Hz), 128.2, 127.5, 121.8, 119.6, 119.2, 116.3, 115.4 (d, J=2.7 Hz), 110.4 (d, J=21.2 Hz), 110.1, 106.41 (d, J=26.2 Hz), 55.8, 49.5. HRMS (ESI) calcd for C₁₇H₁₆FN₂O₂Na, 321.1010 (M+Na)⁺, found 321.1012.

2-(3-(Hydroxymethyl)-1H-indol-1-yl)-N-(3-(trifluoromethyl)phenyl)acetamide (PW01025). Following the similar synthetic procedure that used to prepare compound PW01022, compound PW01025 as a white solid (43 mg, 61%). ¹H NMR (300 MHz, Methanol-d₄) δ 7.97 (s, 1H), 7.73 (dd, J=11.4, 7.9 Hz, 2H), 7.47 (t, J=8.0 Hz, 1H), 7.41-7.31 (m, 2H), 7.27-7.16 (m, 2H), 7.11 (t, J=7.4 Hz, 1H), 4.97 (s, 2H), 4.83 (s, 2H). ¹³C NMR (75 MHz, MeOD) δ 138.8, 137.4, 130.8 (d, J=32.3 Hz), 129.4, 127.7, 127.6, 123.0, 121.8, 120.3 (q, J=3.4 Hz), 119.3, 118.9, 116.2 (d, J=3.8 Hz), 115.5, 108.9, 55.6, 48.9. HRMS (ESI) calcd for C₁₈H₁₆F3N₂O₂ 349.1158 (M+H)⁺, found 349.1154.

2-(3-(Hydroxymethyl)-1H-indol-1-yl)-N-(3-methoxyphenyl)acetamide (PW01026). Following the similar synthetic procedure that used to prepare compound PW01022, compound PW01026 as a white solid (46 mg, 74%). ¹H NMR (300 MHz, Methanol-d₄) δ 7.70 (d, J=7.8 Hz, 1H), 7.35 (d, J=8.1 Hz, 1H), 7.29-7.16 (m, 4H), 7.14-7.01 (m, 2H), 6.68 (dd, J=8.3, 2.6 Hz, 1H), 4.94 (s, 2H), 4.81 (s, 2H), 3.74 (s, 3H). ¹³C NMR (75 MHz, MeOD) δ 167.5, 160.1, 139.0, 137.4, 129.2, 127.7, 127.6, 121.7, 119.2, 118.8, 115.4, 112.0, 109.7, 109.0, 105.7, 55.6, 54.3, 49.0. HRMS (ESI) calcd for C₁₈H₁₉N₂O₃ 311.1390 (M+H)⁺, found 311.1393.

N-(3-Cyanophenyl)-2-(3-(hydroxymethyl)-1H-indol-1-yl)acetamide (PW01027). Following the similar synthetic procedure that used to prepare compound PW01022, compound PW01027 as a white solid (41 mg, 67%). ¹H NMR (300 MHz, Methanol-d₄) δ 7.97 (t, J=1.7 Hz, 1H), 7.78-7.72 (m, 1H), 7.69 (d, J=7.8 Hz, 1H), 7.42 (d, J=7.6 Hz, 2H), 7.33 (d, J=8.1 Hz, 1H), 7.24-7.15 (m, 2H), 7.11 (d, J=7.5 Hz, 1H), 4.96 (s, 2H), 4.81 (s, 2H). ¹³C NMR (75 MHz, MeOD) δ 167.8, 139.0, 137.4, 129.7, 129.7, 127.7, 127.6, 127.3, 124.0, 122.6, 121.8, 119.3, 118.9, 118.0, 115.5, 112.3, 108.9, 55.6, 48.9. HRMS (ESI) calcd for C₁₈H₁₆N₃O₂ 306.1237 (M+H)⁺, found 306.1241.

Ethyl 3-(2-(3-(hydroxymethyl)-1H-indol-1-yl)acetamido)benzoate (PW01028). Following the similar synthetic procedure that used to prepare compound PW01022, compound PW01028 as a white solid (52 mg, 74%). ¹H NMR (300 MHz, DMSO-d₆) δ 10.58 (s, 1H), 8.25 (d, J=2.0 Hz, 1H), 7.85 (d, J=8.1 Hz, 1H), 7.64 (dd, J=12.3, 7.7 Hz, 2H), 7.47 (t, J=7.9 Hz, 1H), 7.39 (d, J=8.1 Hz, 1H), 7.29 (s, 1H), 7.13 (t, J=7.4 Hz, 1H), 7.03 (t, J=7.3 Hz, 1H), 5.03 (s, 2H), 4.81 (t, J=5.3 Hz, 1H), 4.65 (d, J=5.4 Hz, 2H), 4.30 (q, J=7.2 Hz, 2H), 1.31 (t, J=7.0 Hz, 3H). ¹³C NMR (75 MHz, DMSO) δ 167.2, 165.9, 139.5, 137.4, 130.9, 129.8, 128.2, 127.5, 124.5, 124.0, 121.8, 120.0, 119.6, 119.2, 116.3, 110.1, 61.3, 55.8, 49.5, 14.6. HRMS (ESI) calcd for C₂₀H₂₀FN₂O₄Na, 375.1315 (M+Na)⁺, found 375.1321.

2-(3-(Hydroxymethyl)-1H-indol-1-yl)-N-(3-(hydroxymethyl)phenyl)acetamide (PW01029). Following the similar synthetic procedure that used to prepare compound PW01022, compound PW01029 as a white solid (39 mg, 63%). ¹H NMR (300 MHz, DMSO-d₆) δ 10.32 (s, 1H), 7.67-6.94 (m, 9H), 5.17 (t, J=5.7 Hz, 1H), 4.99 (s, 2H), 4.79 (d, J=6.0 Hz, 1H), 4.64 (d, J=5.5 Hz, 2H), 4.45 (d, J=5.6 Hz, 2H). ¹³C NMR (75 MHz, DMSO) δ 166.8, 143.8, 139.0, 137.3, 128.9, 128.3, 127.5, 122.0, 121.7, 119.6, 119.2, 117.9, 117.7, 116.2, 110.1, 63.2, 55.8, 49.6. HRMS (ESI) calcd for C₁₈H₁₉N₂O₃ 311.1390 (M+H)⁺, found 311.1395.

N-(3-(Dimethylamino)phenyl)-2-(3-(hydroxymethyl)-1H-indol-1-yl)acetamide (PW01030). Following the similar synthetic procedure that used to prepare compound PW01022, compound PW01030 as a white solid (27 mg, 42%). ¹H NMR (300 MHz, DMSO-d₆) δ 10.16 (s, 1H), 7.61 (d, J=7.6 Hz, 1H), 7.38 (d, J=8.1 Hz, 1H), 7.27 (s, 1H), 7.07 (dq, J=18.0, 7.6, 7.1 Hz, 4H), 6.87 (d, J=8.0 Hz, 1H), 6.44 (d, J=8.4 Hz, 1H), 4.97 (s, 2H), 4.79 (t, J=5.0 Hz, 1H), 4.64 (d, J=5.4 Hz, 2H), 2.85 (s, 6H). ¹³C NMR (75 MHz, DMSO) δ 166.7, 151.3, 139.9, 137.3, 129.6, 128.3, 127.5, 121.7, 119.6, 119.2, 116.1, 110.1, 108.4, 107.8, 103.6, 55.7, 49.6, 40.5. HRMS (ESI) calcd for C₁₉H₂₂N₃O₂ 324.1707 (M+H)⁺, found 324.1713.

N-(3-Ethylphenyl)-2-(3-(hydroxymethyl)-1H-indol-1-yl)acetamide (PW01031). Following the similar synthetic procedure that used to prepare compound PW01022, compound PW01031 as a white solid (52 mg, 84%). ¹H NMR (300 MHz, DMSO-d₆) δ 10.27 (s, 1H), 7.61 (d, J=7.8 Hz, 1H), 7.40 (dd, J=16.7, 10.5 Hz, 3H), 7.28 (s, 1H), 7.16 (dt, J=25.0, 7.8 Hz, 2H), 7.02 (t, J=7.3 Hz, 1H), 6.91 (d, J=7.5 Hz, 1H), 4.98 (s, 2H), 4.79 (d, J=5.4 Hz, 1H), 4.65 (d, J=5.4 Hz, 2H), 2.56 (d, J=7.4 Hz, 2H), 1.15 (t, J=7.6 Hz, 3H). ¹³C NMR (75 MHz, DMSO) δ 166.8, 144.8, 139.2, 137.3, 129.2, 128.3, 127.5, 123.5, 121.7, 119.6, 119.2, 119.0, 117.0, 116.2, 110.1, 55.8, 49.6, 28.7, 15.9. HRMS (ESI) calcd for C₁₉H₂₂N₃O₂ 331.1417 (M+H)⁺, found 331.1424.

2-(3-(Hydroxymethyl)-1H-indol-1-yl)-N-(o-tolyl)acetamide (PW01032). Following the similar synthetic procedure that used to prepare compound PW01022, compound PW01032 as a white solid (35 mg, 59%). ¹H NMR (300 MHz, Methanol-d₄) δ 7.73 (d, J=7.8 Hz, 1H), 7.47-7.35 (m, 2H), 7.30 (s, 1H), 7.24 (t, J=7.6 Hz, 1H), 7.20-7.09 (m, 4H), 5.03 (s, 2H), 4.83 (s, 2H), 2.05 (s, 3H). ¹³C NMR (75 MHz, MeOD) δ 168.1, 137.2, 134.9, 132.2, 130.1, 127.7, 127.6, 126.0, 125.9, 124.9, 121.9, 119.5, 119.0, 115.9, 109.1, 55.6, 49.0, 16.4. HRMS (ESI) calcd for C₁₈H₁₈N₂O₂Na, 317.1260 (M+Na)⁺, found 317.1266.

2-(3-(Hydroxymethyl)-1H-indol-1-yl)-N-(p-tolyl)acetamide (PW01033). Following the similar synthetic procedure that used to prepare compound PW01022, compound PW01033 as a white solid (40 mg, 68%). ¹H NMR (300 MHz, Methanol-d₄) δ 7.71 (d, J=8.0 Hz, 1H), 7.38 (t, J=9.6 Hz, 3H), 7.30-7.04 (m, 5H), 4.97 (s, 2H), 4.83 (s, 2H), 2.30 (s, 3H). ¹³C NMR (75 MHz, MeOD) δ 167.3, 137.4, 135.2, 134.0, 128.9, 127.7, 121.7, 120.0, 119.2, 118.8, 115.5, 109.0, 55.6, 49.0, 19.5. HRMS (ESI) calcd for C₁₈H₁₈N₂O₂Na, 317.1260 (M+Na)⁺, found 317.1266.

N-(3-Chloro-5-fluorophenyl)-2-(3-(hydroxymethyl)-1H-indol-1-yl)acetamide (PW01034). Following the similar synthetic procedure that used to prepare compound PW01022, compound PW01034 as a white solid (31 mg, 47%). ¹H NMR (300 MHz, Methanol-d₄) δ 7.70 (d, J=7.8 Hz, 1H), 7.41 (dt, J=12.4, 2.1 Hz, 2H), 7.33 (d, J=8.1 Hz, 1H), 7.26-7.16 (m, 2H), 7.15-7.07 (m, 1H), 6.92 (dd, J=8.5, 2.1 Hz, 1H), 4.97 (s, 2H), 4.82 (s, 2H). ¹³C NMR (75 MHz, MeOD) δ 167.8, 164.4, 161.1, 140.6 (d, J=12.3 Hz), 137.4, 134.8 (d, J=12.5 Hz), 127.6, 127.5, 121.8, 119.3, 118.8, 115.5, 115.3 (d, J=3.3 Hz), 111.0 (d, J=25.5 Hz), 108.9, 105.1 (d, J=26.5 Hz), 55.6, 48.9. HRMS (ESI) calcd for C₁₇H₁₅ClFN₂O₂ 333.0801 (M+H)⁺, found 333.0809.

N-(3-Chloro-4-fluorophenyl)-2-(3-(hydroxymethyl)-1H-indol-1-yl)acetamide (PW01035). Following the similar synthetic procedure that used to prepare compound PW01022, compound PW01035 as a white solid (28 mg, 42%). ¹H NMR (300 MHz, Methanol-d₄) δ 7.80 (dd, J=6.7, 2.6 Hz, 1H), 7.71 (dt, J=7.8, 1.0 Hz, 1H), 7.43 (ddd, J=9.0, 4.2, 2.6 Hz, 1H), 7.38-7.32 (m, 1H), 7.26 (s, 1H), 7.24-7.15 (m, 2H), 7.11 (ddd, J=8.0, 7.0, 1.1 Hz, 1H), 4.98 (s, 2H), 4.82 (s, 2H). ¹³C NMR (75 MHz, MeOD) δ 167.6, 156.2, 152.9, 137.4, 135.0, 127.6 (d, J=5.5 Hz), 121.8 (d, J=3.8 Hz), 120.2 (d, J=18.6 Hz), 119.9 (d, J=7.0 Hz), 119.3, 118.8, 116.2 (d, J=22.2 Hz), 115.5, 108.9, 55.6, 48.9. HRMS (ESI) calcd for C₁₇H₁₅ClFN₂O₂ 333.0801 (M+H)⁺, found 333.0809.

N-(4-Chloro-3-fluorophenyl)-2-(3-(hydroxymethyl)-1H-indol-1-yl)acetamide (PW01036). Following the similar synthetic procedure that used to prepare compound PW01022, compound PW01036 as a white solid (37 mg, 56%). ¹H NMR (300 MHz, Methanol-d₄) δ 7.75-7.63 (m, 2H), 7.46-7.32 (m, 2H), 7.31-7.24 (m, 2H), 7.20 (ddd, J=8.3, 7.0, 1.3 Hz, 1H), 7.11 (ddd, J=8.0, 7.0, 1.1 Hz, 1H), 5.00 (s, 2H), 4.83 (s, 2H). ¹³C NMR (75 MHz, MeOD) δ 167.7, 166.6, 137.4, 130.2, 127.6 (d, J=6.4 Hz), 121.8, 119.2, 118.8, 116.1 (d, J=3.5 Hz), 115.5, 108.9, 108.0 (d, J=26.0 Hz), 55.6, 48.9. HRMS (ESI) calcd for C₁₇H₁₅ClFN₂O₂ 333.0801 (M+H)⁺, found 333.0809.

N-(3-Chloro-4-(trifluoromethyl)phenyl)-2-(3-(hydroxymethyl)-1H-indol-1-yl)acetamide (PW01037). Following the similar synthetic procedure that used to prepare compound PW01022, compound PW01037 as a white solid (33 mg, 43%). ¹H NMR (300 MHz, Methanol-d₄) δ 7.95 (d, J=1.9 Hz, 1H), 7.71 (dd, J=8.4, 1.8 Hz, 2H), 7.63 (dd, J=8.8, 2.0 Hz, 1H), 7.38-7.32 (m, 1H), 7.27 (s, 1H), 7.21 (ddd, J=8.2, 7.0, 1.3 Hz, 1H), 7.12 (ddd, J=8.0, 7.0, 1.1 Hz, 1H), 5.04 (s, 2H), 4.83 (s, 2H). ¹³C NMR (75 MHz, MeOD) δ 168.0, 166.6, 142.6, 137.4, 132.3-132.1 (m), 127.9 (d, J=5.4 Hz), 127.6 (2C), 124.8, 123.0, 122.8 (d, J=32.0 Hz), 121.5, 121.2, 119.3, 118.8, 117.2, 115.6, 108.9, 55.6, 48.9. HRMS (ESI) calcd for C₁₈H₁₅ClF₃N₂O₂ 383.0769 (M+H)⁺, found 383.0776.

N-(3,5-Dichlorophenyl)-2-(3-(hydroxymethyl)-1H-indol-1-yl)acetamide (PW01038). Following the similar synthetic procedure that used to prepare compound PW01022, compound PW01038 as a white solid (57 mg, 81%). ¹H NMR (300 MHz, Methanol-d₄) δ 7.70 (dd, J=7.8, 1.2 Hz, 1H), 7.60 (d, J=1.8 Hz, 2H), 7.34 (d, J=8.1 Hz, 1H), 7.26-7.17 (m, 2H), 7.16-7.06 (m, 2H), 4.98 (s, 2H), 4.82 (s, 2H). ¹³C NMR (75 MHz, MeOD) δ 167.8, 140.3, 137.4, 134.8, 127.6, 127.5, 123.4, 121.8, 119.3, 118.8, 117.8, 115.6, 108.9, 55.6, 48.9. HRMS (ESI) calcd for C₁₇H₁₄Cl₂N₂O₂Na, 371.0325 (M+Na)⁺, found 371.0332.

N-(3,4-Difluorophenyl)-2-(3-(hydroxymethyl)-1H-indol-1-yl)acetamide (PW01039). Following the similar synthetic procedure that used to prepare compound PW01022, compound PW01039 as a white solid (36 mg, 57%). ¹H NMR (300 MHz, Methanol-d₄) δ 7.68 (dd, J=13.7, 7.0 Hz, 2H), 7.22 (ddt, J=28.1, 19.1, 8.0 Hz, 6H), 4.97 (s, 2H), 4.82 (s, 2H). ¹³C NMR (75 MHz, MeOD) δ 167.5, 137.4, 127.6 (d, J=6.5 Hz), 121.8, 119.2, 118.8, 116.8 (d, J=18.9 Hz), 115.8 (dd, J=5.6, 3.3 Hz), 115.5, 109.1 (d, J=22.0 Hz), 108.9, 55.6, 48.9. HRMS (ESI) calcd for C₁₇H₁₄F2N₂O₂Na, 339.0916 (M+Na)⁺, found 339.0916.

N-(3,5-Difluorophenyl)-2-(3-(hydroxymethyl)-1H-indol-1-yl)acetamide (PW01040). Following the similar synthetic procedure that used to prepare compound PW01022, compound PW01040 as a white solid (24 mg, 38%). ¹H NMR (300 MHz, Methanol-d₄) δ 7.76-7.67 (m, 1H), 7.38-7.32 (m, 1H), 7.30-7.17 (m, 4H), 7.11 (td, J=7.4, 1.1 Hz, 1H), 6.68 (tt, J=9.2, 2.4 Hz, 1H), 5.00 (s, 2H), 4.83 (s, 2H). ¹³C NMR (75 MHz, MeOD) δ 167.7, 164.8 (d, J=14.5 Hz), 161.5 (d, J=14.8 Hz), 140.7, 137.4, 127.6 (2C), 121.8, 119.2, 118.8, 115.5, 108.9, 102.4 (d, J=29.4 Hz), 98.6 (t, J=26.1 Hz), 55.6, 48.9. HRMS (ESI) calcd for C₁₇H₁₅F2N₂O₂ 317.1096 (M+H)⁺, found 317.1100.

N-(3-Fluoro-5-(trifluoromethyl)phenyl)-2-(3-(hydroxymethyl)-1H-indol-1-yl)acetamide (PW01041). Following the similar synthetic procedure that used to prepare compound PW01022, compound PW01041 as a white solid (26 mg, 36%). ¹H NMR (300 MHz, Methanol-d₄) δ 7.79-7.66 (m, 3H), 7.35 (d, J=8.1 Hz, 1H), 7.26 (s, 1H), 7.24-7.08 (m, 3H), 5.02 (s, 2H), 4.83 (s, 2H). ¹³C NMR (75 MHz, MeOD) δ 167.9, 164.3, 161.1, 141.6-140.5 (m), 140.8, 137.4, 127.6, 127.6, 121.8, 119.3, 118.8, 115.6, 112.0-111.7 (m), 110.0, 109.6, 108.9, 107.6-107.3 (m), 107.10 (d, J=3.7 Hz), 55.6, 48.9. HRMS (ESI) calcd for C₁₈H₁₄F₄N₂O₂Na, 389.0884 (M+Na)⁺, found 389.0891.

Methyl 3-(2-(3-(hydroxymethyl)-1H-indol-1-yl)acetamido)benzoate (PW01050). Following the similar synthetic procedure that used to prepare compound PW01022, compound PW01050 as a white solid (50 mg, 74%). ¹H NMR (300 MHz, DMSO-d₆) δ 10.57 (s, 1H), 8.27 (s, 1H), 7.83 (d, J=8.1 Hz, 1H), 7.63 (dd, J=12.3, 7.8 Hz, 2H), 7.47 (t, J=8.0 Hz, 1H), 7.39 (d, J=8.2 Hz, 1H), 7.29 (s, 1H), 7.13 (t, J=7.6 Hz, 1H), 7.03 (t, J=7.5 Hz, 1H), 5.02 (s, 2H), 4.80 (s, 1H), 4.65 (d, J=5.2 Hz, 2H), 3.83 (s, 3H). ¹³C NMR (75 MHz, DMSO) δ 167.2, 166.4, 139.5, 137.4, 130.6, 129.8, 128.2, 127.5, 124.5, 124.0, 121.8, 120.1, 119.6, 119.2, 116.3, 110.1, 55.8, 52.6, 49.5. HRMS (ESI) calcd for C₁₉H₁₉N₂O₄ 339.1339 (M+H)⁺, found 339.1343.

Methyl 2-fluoro-5-(2-(3-(hydroxymethyl)-1H-indol-1-yl)acetamido)benzoate (PW01051). Following the similar synthetic procedure that used to prepare compound PW01022, compound PW01051 as a white solid (52 mg, 73%). ¹H NMR (300 MHz, DMSO-d₆) δ 10.59 (s, 1H), 8.19 (dd, J=6.5, 2.8 Hz, 1H), 7.90-7.77 (m, 1H), 7.62 (d, J=7.8 Hz, 1H), 7.43-7.30 (m, 2H), 7.29 (s, 1H), 7.13 (t, J=7.8 Hz, 1H), 7.03 (t, J=7.4 Hz, 1H), 5.01 (s, 2H), 4.81 (t, J=5.3 Hz, 1H), 4.65 (d, J=5.3 Hz, 2H), 3.84 (s, 3H). ¹³C NMR (75 MHz, DMSO) δ 167.1, 164.2 (d, J=3.4 Hz), 157.2 (d, J=254.2 Hz), 137.4, 135.5 (d, J=3.0 Hz), 128.2, 127.5, 125.9 (d, J=8.7 Hz), 122.2, 121.8, 119.6, 119.2, 118.4 (d, J=11.0 Hz), 118.0 (d, J=23.6 Hz), 116.3, 110.1, 55.8, 52.9, 49.4. HRMS (ESI) calcd for C₁₉H₁₇FN₂O₄Na, 379.1065 (M+Na)⁺, found 379.1071.

3-(2-(3-(Hydroxymethyl)-1H-indol-1-yl)acetamido)benzamide (PW01053). Following the similar synthetic procedure that used to prepare compound PW01022, compound PW01053 as a white solid (23 mg, 36%). ¹H NMR (300 MHz, DMSO-d₆) δ 10.47 (s, 1H), 8.03 (s, 1H), 7.92 (s, 1H), 7.77 (d, J=8.1 Hz, 1H), 7.58 (dd, J=18.7, 7.8 Hz, 2H), 7.35 (dd, J=25.1, 8.8 Hz, 4H), 7.13 (t, J=7.5 Hz, 1H), 7.03 (t, J=7.3 Hz, 1H), 5.01 (s, 2H), 4.81 (d, J=5.3 Hz, 1H), 4.65 (d, J=5.4 Hz, 2H). ¹³C NMR (75 MHz, DMSO) δ 168.2, 167.0, 139.2, 137.3, 135.6, 129.1, 128.3, 127.5, 122.7, 122.3, 121.7, 119.6, 119.2, 119.2, 116.2, 110.1, 55.8, 49.5. HRMS (ESI) calcd for C₁₈H₁₈N₃O₃ 324.1343 (M+H)⁺, found 324.1347.

N-((3s,5s,7s)-Adamantan-1-yl)-2-(3-(hydroxymethyl)-1H-indol-1-yl)acetamide (PW01043). Following the similar synthetic procedure that used to prepare compound PW01022, compound PW01043 as a white solid (51 mg, 76%). ¹H NMR (300 MHz, Methanol-d₄) δ 7.69 (d, J=7.8 Hz, 1H), 7.31 (d, J=8.1 Hz, 1H), 7.24-7.15 (m, 3H), 7.14-7.07 (m, 1H), 4.81 (s, 2H), 4.72 (s, 2H), 2.06-1.98 (m, 9H), 1.71 (d, J=3.0 Hz, 6H). ¹³C NMR (75 MHz, MeOD) δ 168.1, 166.6, 137.1, 127.6, 121.7, 119.2, 118.8, 115.4, 109.0, 55.6, 51.9, 49.2, 40.9, 36.0, 29.5. HRMS (ESI) calcd for C₂₁H₂₆N₂O₂Na, 361.1886 (M+Na)⁺, found 361.1890.

N-((1S,2S,4R)-Bicyclo[2.2.1]heptan-2-yl)-2-(3-(hydroxymethyl)-1H-indol-1-yl)acetamide (PW01044). Following the similar synthetic procedure that used to prepare compound PW01022, compound PW01044 as a white solid (26 mg, 44%). ¹H NMR (300 MHz, Methanol-d₄) δ 7.69 (dt, J=7.8, 1.0 Hz, 1H), 7.31 (dt, J=8.2, 1.0 Hz, 1H), 7.26-7.14 (m, 3H), 7.10 (ddd, J=8.1, 7.0, 1.2 Hz, 1H), 4.80 (s, 2H), 4.77 (s, 2H), 3.65-3.59 (m, 1H), 2.26 (t, J=3.8 Hz, 1H), 2.16 (d, J=3.8 Hz, 1H), 1.72 (ddd, J=13.1, 8.1, 2.4 Hz, 1H), 1.50 (tdd, J=6.0, 5.0, 4.5, 2.5 Hz, 2H), 1.39 (dt, J=10.0, 1.9 Hz, 1H), 1.26-1.12 (m, 4H). ¹³C NMR (75 MHz, MeOD) δ 168.3, 137.2, 127.6, 121.6, 119.2, 118.8, 115.4, 109.0, 55.6, 53.0, 48.6, 42.1, 38.7, 35.5, 34.7, 27.8, 25.9. HRMS (ESI) calcd for C₁₈H₂₂N₂O₂Na 321.1573 (M+Na)⁺, found 321.1582.

N-Cyclohexyl-2-(3-(hydroxymethyl)-1H-indol-1-yl)acetamide (PW01045). Following the similar synthetic procedure that used to prepare compound PW01022, compound PW01045 as a white solid (33 mg, 58%). ¹H NMR (300 MHz, DMSO-d₆) δ 8.06 (d, J=7.8 Hz, 1H), 7.59 (d, J=7.8 Hz, 1H), 7.31 (d, J=8.1 Hz, 1H), 7.20 (s, 1H), 7.11 (ddd, J=8.3, 7.0, 1.3 Hz, 1H), 7.05-6.96 (m, 1H), 4.78 (s, 1H), 4.72 (s, 2H), 4.63 (s, 2H), 3.61-3.46 (m, 1H), 1.71 (d, J=31.4 Hz, 4H), 1.54 (d, J=11.1 Hz, 1H), 1.22 (q, J=12.8, 12.1 Hz, 5H). ¹³C NMR (75 MHz, DMSO) δ 166.7, 137.1, 128.2, 127.5, 121.6, 119.5, 119.1, 115.9, 110.1, 55.7, 49.2, 48.1, 32.8, 25.6, 24.8. HRMS (ESI) calcd for C₁₇H₂₃N₂O₂ 287.1754 (M+H)⁺, found 287.1757.

N-Cyclopentyl-2-(3-(hydroxymethyl)-1H-indol-1-yl)acetamide (PW01046). Following the similar synthetic procedure that used to prepare compound PW01022, compound PW01046 as a white solid (30 mg, 56%). ¹H NMR (300 MHz, Chloroform-d and Methanol-d₄) δ 7.69 (d, J=7.8 Hz, 1H), 7.30-7.10 (m, 3H), 7.00 (s, 1H), 5.87 (d, J=6.2 Hz, 1H), 4.79 (s, 2H), 4.63 (s, 2H), 4.11 (q, J=7.1 Hz, 1H), 1.92-1.72 (m, 2H), 1.50-1.39 (m, 4H), 1.21-1.06 (m, 2H). ¹³C NMR (75 MHz, Chloroform-d and Methanol-d₄) δ 168.1, 136.8, 127.4, 127.0, 122.8, 120.3, 119.5, 116.7, 109.2, 56.4, 51.3, 49.7, 32.5, 32.5, 23.4, 23.4. HRMS (ESI) calcd for C₁₆H₂₁N₂O₂ 273.1598 (M+H)⁺, found 273.1602.

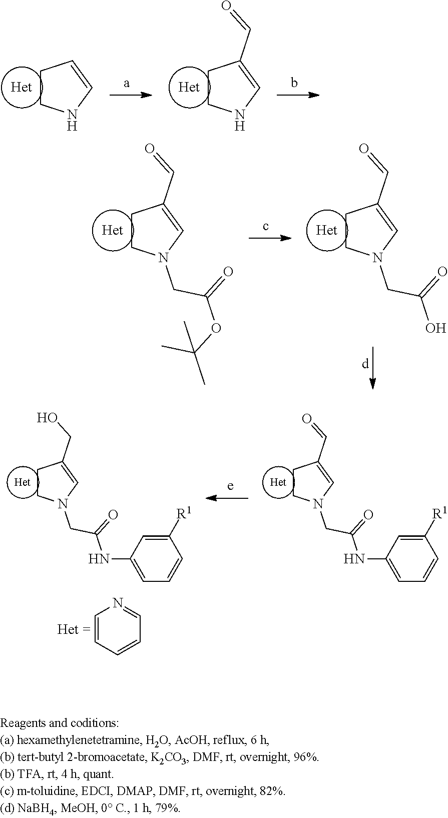

Azaindole-3-carbaldehyde. To a mixture of hexamethylenetetramine (2.1 g, 15 mmol) and azaindole (1.2 g, 10 mmol) in water (6 mL) was added acetic acid (3 mL) at room temperature. The mixture was heated to reflux and stirred at reflux for 2 h until TLC indicated the end of reaction. The mixture was diluted with water at room temperature and cooled to 0° C. The precipitates were collected by filtration, washed with cold water, and dried to get the compound.

1H-Pyrrolo[2,3-b]pyridine-3-carbaldehyde. Yellow solid (760 mg, 52%). ¹H NMR (300 MHz, DMSO-d₆) δ 12.69 (s, 1H), 9.92 (s, 1H), 8.46 (s, 1H), 8.43-8.33 (m, 2H), 7.28 (dd, J=7.8, 4.7 Hz, 1H).

1H-Pyrrolo[3,2-b]pyridine-3-carbaldehyde. Yellow solid (730 mg, 50%). ¹H NMR (300 MHz, DMSO-d₆) δ 12.35 (s, 1H), 10.18 (s, 1H), 8.50 (dd, J=4.7, 1.5 Hz, 1H), 8.42 (s, 1H), 7.91 (dd, J=8.2, 1.5 Hz, 1H), 7.27 (dd, J=8.3, 4.6 Hz, 1H).

1H-Pyrrolo[3,2-c]pyridine-3-carbaldehyde. Yellow solid (804 mg, 55%). ¹H NMR (300 MHz, DMSO-d₆) δ 12.42 (s, 1H), 9.99 (s, 1H), 9.28 (d, J=1.1 Hz, 1H), 8.42 (s, 1H), 8.34 (d, J=5.7 Hz, 1H), 7.52 (dd, J=5.6, 1.2 Hz, 1H).

1H-Pyrrolo[2,3-c]pyridine-3-carbaldehyde. Yellow solid (570 mg, 39%). ¹H NMR (300 MHz, DMSO-d₆) δ 12.55 (s, 1H), 10.00 (s, 1H), 8.87 (d, J=1.2 Hz, 1H), 8.50 (s, 1H), 8.32 (d, J=5.4 Hz, 1H), 7.99 (dd, J=5.4, 1.2 Hz, 1H).

tert-Butyl 2-(3-formyl-1H-pyrrolo[2,3-b]pyridin-1-yl)acetate (PW01009). Following the similar synthetic procedure that used to prepare compound PW0985, compound PW01009 as a light yellow solid (410 mg, 79%). ¹H NMR (300 MHz, Chloroform-d) δ 10.03 (s, 1H), 8.58 (dd, J=7.8, 1.6 Hz, 1H), 8.42 (dd, J=4.8, 1.6 Hz, 1H), 7.95 (s, 1H), 7.34-7.24 (m, 2H), 5.06 (s, 2H), 1.50 (s, 9H).

tert-Butyl 2-(3-formyl-1H-pyrrolo[3,2-b]pyridin-1-yl)acetate (PW01056). Following the similar synthetic procedure that used to prepare compound PW0985, compound PW01056 as a light yellow solid (630 mg, 81%). ¹H NMR (300 MHz, DMSO-d₆) δ 10.19 (s, 1H), 8.54 (dd, J=4.7, 1.4 Hz, 1H), 8.43 (s, 1H), 8.00 (dd, J=8.4, 1.4 Hz, 1H), 7.32 (dd, J=8.4, 4.6 Hz, 1H), 5.22 (s, 2H), 1.42 (d, J=2.2 Hz, 9H).

tert-Butyl 2-(3-formyl-1H-pyrrolo[3,2-c]pyridin-1-yl)acetate (PW01065). Following the similar synthetic procedure that used to prepare compound PW0985, compound PW01065 as a light brown solid (600 mg, 77%). ¹H NMR (300 MHz, DMSO-d₆) δ 10.00 (s, 1H), 9.30 (d, J=1.0 Hz, 1H), 8.40 (d, J=6.2 Hz, 2H), 7.61 (dd, J=5.8, 1.1 Hz, 1H), 5.23 (s, 2H), 1.43 (s, 9H).

tert-Butyl 2-(3-formyl-1H-pyrrolo[2,3-c]pyridin-1-yl)acetate (PW01066). Following the similar synthetic procedure that used to prepare compound PW0985, compound PW01065 as a light brown solid (560 mg, 72%). ¹H NMR (300 MHz, DMSO-d₆) δ 10.00 (s, 1H), 8.92 (d, J=1.1 Hz, 1H), 8.46 (s, 1H), 8.37 (d, J=5.4 Hz, 1H), 8.01 (dd, J=5.4, 1.1 Hz, 1H), 5.31 (s, 2H), 1.43 (s, 9H).

2-(3-Formyl-1H-pyrrolo[2,3-b]pyridin-1-yl)acetic acid (PW01012). Following the similar synthetic procedure that used to prepare compound PW0985B, compound PW01012 as a yellow solid. ¹H NMR (300 MHz, DMSO-d₆) δ 13.18 (s, 1H), 9.96 (s, 1H), 8.51 (s, 1H), 8.44 (dd, J=7.8, 1.6 Hz, 1H), 8.40 (dd, J=4.8, 1.7 Hz, 1H), 7.35 (dd, J=7.8, 4.8 Hz, 1H), 5.17 (s, 2H).

2-(3-Formyl-1H-pyrrolo[3,2-b]pyridin-1-yl)acetic acid (PW01059). Following the similar synthetic procedure that used to prepare compound PW0985B, compound PW01059 as a yellow solid. ¹H NMR (300 MHz, DMSO-d₆) δ 10.16 (s, 1H), 8.72 (s, 1H), 8.65 (dd, J=5.2, 1.3 Hz, 1H), 8.49 (dd, J=8.4, 1.2 Hz, 1H), 7.61 (dd, J=8.4, 5.2 Hz, 1H), 5.34 (s, 2H).

2-(3-Formyl-1H-pyrrolo[3,2-c]pyridin-1-yl)acetic acid (PW01067). Following the similar synthetic procedure that used to prepare compound PW0985B, compound PW01067 as a yellow solid. ¹H NMR (300 MHz, DMSO-d₆) δ 10.14 (s, 1H), 9.50 (s, 1H), 8.86 (s, 1H), 8.72 (d, J=6.7 Hz, 1H), 8.36 (d, J=6.8 Hz, 1H), 5.46 (s, 2H).

2-(3-Formyl-1H-pyrrolo[2,3-c]pyridin-1-yl)acetic acid (PW01082). Following the similar synthetic procedure that used to prepare compound PW0985B, compound PW01082 as a yellow solid.

2-(3-(Hydroxymethyl)-1H-pyrrolo[2,3-b]pyridin-1-yl)-N-(m-tolyl)acetamide (PW01016). In a round-bottom flask, compound PW01012 (41 mg, 0.2 mmol) was dissolved in 2 mL of anhydrous DMF, and the solution was cooled to 0° C. with an ice bath. m-Toluidine (43 mg, 0.4 mmol), EDCI (77 mg, 0.4 mmol), DMAP (49 mg, 0.4 mmol) were added to the solution at 0° C. Then removed the ice bath and the mixture solution was stirred at rt overnight until TLC indicated the end of reaction. Then poured the solution into ice water (10 mL) and extracted with EtOAc (10 mL×3). The organic phase was washed with brine, dried over Na₂SO₄, and then concentrated under reduced pressure. The residue was used directly to the next step. In a round-bottom flask, the residue was dissolved in 2 mL of MeOH, and the solution was cooled to 0° C. with an ice bath. NaBH₄ (8 mg, 0.4 mmol) was slowly added to solution in a small portion and the solution was continually stirred for 1 h until TLC indicated the end of reaction. Then poured the solution into ice water (10 mL) and extracted with EtOAc (10 mL×3). The organic phase was washed with brine, dried over Na₂SO₄, and then concentrated under reduced pressure. The residue was purified by flash column chromatography (DCM/MeOH=50/1) to get the PW01016 as a white solid (22 mg, 39%). ¹H NMR (300 MHz, DMSO-d₆) δ 10.27 (s, 1H), 8.21 (d, J=4.6 Hz, 1H), 8.03 (d, J=7.8 Hz, 1H), 7.40 (dd, J=20.6, 9.1 Hz, 3H), 7.25-7.03 (m, 2H), 6.87 (d, J=7.5 Hz, 1H), 5.09 (s, 2H), 4.93 (t, J=5.4 Hz, 1H), 4.66 (d, J=5.5 Hz, 2H), 2.26 (s, 3H). ¹³C NMR (75 MHz, DMSO) δ 166.8, 166.6, 148.2, 142.7, 139.2, 138.4, 129.1, 128.4, 127.9, 124.5, 120.1, 119.6, 116.7, 115.8, 114.6, 55.8, 47.3, 21.6. HRMS (ESI) calcd for C₁₇H₁₈N₃O₂ 296.1394 (M+H)⁺, found 296.1399.

2-(3-(Hydroxymethyl)-1H-pyrrolo[3,2-b]pyridin-1-yl)-N-(m-tolyl)acetamide (PW01061). Following the similar synthetic procedure that used to prepare compound PW01016, compound PW01061 as a white solid (33 mg, 56%). ¹H NMR (300 MHz, DMSO-d₆) δ 10.29 (s, 1H), 8.42-8.24 (m, 1H), 7.80 (d, J=8.2 Hz, 1H), 7.57 (s, 1H), 7.47-7.28 (m, 2H), 7.15 (dt, J=12.9, 6.3 Hz, 2H), 6.87 (d, J=7.5 Hz, 1H), 5.05 (s, 2H), 4.85 (s, 1H), 4.73 (d, J=5.0 Hz, 2H), 2.25 (s, 3H). ¹³C NMR (75 MHz, DMSO) δ 166.5, 144.8, 142.4, 139.1, 138.5, 131.8, 130.1, 129.1, 124.7, 120.2, 117.5, 116.8, 116.7, 54.5, 49.7, 21.6. HRMS (ESI) calcd for C₁₇H₁₈N₃O₂ 296.1394 (M+H)⁺, found 296.1395.

Ethyl 3-(2-(3-(hydroxymethyl)-1H-pyrrolo[3,2-b]pyridin-1-yl)acetamido)benzoate (PW01062). Following the similar synthetic procedure that used to prepare compound PW01016, compound PW01062 as a white solid (26 mg, 37%). ¹H NMR (300 MHz, DMSO-d₆) δ 10.61 (s, 1H), 8.39-8.19 (m, 2H), 7.82 (d, J=8.0 Hz, 2H), 7.70-7.54 (m, 2H), 7.46 (t, J=8.1 Hz, 1H), 7.12 (dd, J=8.3, 4.7 Hz, 1H), 5.10 (s, 2H), 4.86 (t, J=5.5 Hz, 1H), 4.73 (d, J=4.8 Hz, 2H), 4.29 (q, J=7.1 Hz, 2H), 1.29 (t, J=7.2 Hz, 3H). ¹³C NMR (75 MHz, DMSO) δ 167.0, 165.9, 144.8, 142.4, 139.5, 131.7, 130.9, 130.2, 129.8, 124.6, 124.0, 120.0, 117.5, 116.8, 116.7, 61.3, 54.5, 49.6, 14.6. HRMS (ESI) calcd for C₁₉H₁₉N₃O₄Na, 376.1268 (M+Na)⁺, found 376.1274.

2-(3-(Hydroxymethyl)-1H-pyrrolo[3,2-c]pyridin-1-yl)-N-(m-tolyl)acetamide (PW01076). Following the similar synthetic procedure that used to prepare compound PW01016, compound PW01076 as a white solid (44 mg, 75%). ¹H NMR (300 MHz, DMSO-d₆) δ 10.30 (s, 1H), 8.88 (s, 1H), 8.19 (d, J=5.8 Hz, 1H), 7.49-7.29 (m, 4H), 7.19 (t, J=7.8 Hz, 1H), 6.88 (d, J=7.5 Hz, 1H), 5.03 (d, J=3.8 Hz, 3H), 4.69 (d, J=5.3 Hz, 2H), 2.26 (s, 3H). ¹³C NMR (75 MHz, DMSO) δ 166.3, 142.6, 141.0, 140.8, 139.0, 138.5, 129.1, 128.9, 124.7, 124.2, 120.1, 116.8, 116.5, 105.8, 55.5, 49.2, 21.6. HRMS (ESI) calcd for C₁₇H₁₈N₃O₂ 296.1394 (M+H)⁺, found 296.1395.

Ethyl 3-(2-(3-(Hydroxymethyl)-1H-pyrrolo[3,2-c]pyridin-1-yl)acetamido)benzoate (PW01074). Following the similar synthetic procedure that used to prepare compound PW01016, compound PW01074 as a white solid (43 mg, 61%). ¹H NMR (300 MHz, DMSO-d₆) δ 10.61 (s, 1H), 8.89 (s, 1H), 8.30-8.16 (m, 2H), 7.89-7.78 (m, 1H), 7.66 (dt, J=7.8, 1.3 Hz, 1H), 7.53-7.40 (m, 2H), 7.36 (s, 1H), 5.08 (s, 2H), 5.03 (t, J=5.4 Hz, 1H), 4.70 (d, J=5.4 Hz, 2H), 4.30 (q, J=7.1 Hz, 2H), 1.30 (t, J=7.1 Hz, 3H). ¹³C NMR (75 MHz, DMSO) δ 166.8, 165.9, 142.6, 141.0, 140.8, 139.5, 130.9, 129.8, 128.9, 124.6, 124.2, 124.0, 120.0, 116.5, 105.8, 61.3, 55.5, 49.1, 14.6. HRMS (ESI) calcd for C₁₉H₂₀N₃O₄ 354.1448 (M+H)⁺, found 354.1460.

2-(3-(Hydroxymethyl)-1H-pyrrolo[2,3-c]pyridin-1-yl)-N-(m-tolyl)acetamide (PW01083). Following the similar synthetic procedure that used to prepare compound PW01016, compound PW01083 as a white solid (24 mg, 41%). ¹H NMR (300 MHz, DMSO) δ 10.35 (s, 1H), 8.79 (s, 1H), 8.14 (s, 1H), 7.83-7.09 (m, 5H), 6.90 (s, 1H), 5.45-4.84 (m, 3H), 4.66 (s, 2H), 2.27 (s, 3H). ¹³C NMR (75 MHz, DMSO) δ 166.4, 139.0, 138.5, 138.1, 134.4, 133.6, 132.0, 131.8, 129.2, 124.7, 120.2, 116.8, 115.9, 114.2, 55.3, 49.7, 21.6. HRMS (ESI) calcd for C₁₇H₁₈N₃O₂ 296.1394 (M+H)⁺, found 296.1395.

Ethyl 3-(2-(3-(hydroxymethyl)-1H-pyrrolo[2,3-c]pyridin-1-yl)acetamido)benzoate (PW01084). Following the similar synthetic procedure that used to prepare compound PW01016, compound PW01084 as a white solid (28 mg, 40%). ¹H NMR (300 MHz, DMSO) δ 10.62 (s, 1H), 8.77 (s, 1H), 8.37-7.99 (m, 2H), 7.94-7.36 (m, 5H), 5.33-4.84 (m, 3H), 4.63 (s, 2H), 4.27 (s, 2H), 1.27 (s, 3H). ¹³C NMR (75 MHz, DMSO) δ 166.9, 165.9, 139.5, 138.1, 134.4, 133.6, 132.0, 131.8, 130.9, 129.8, 124.6, 124.0, 120.1, 116.0, 114.2, 61.3, 55.3, 49.6, 14.6. HRMS (ESI) calcd for C₁₉H₂₀N₃O₄ 354.1448 (M+H)⁺, found 354.1460.

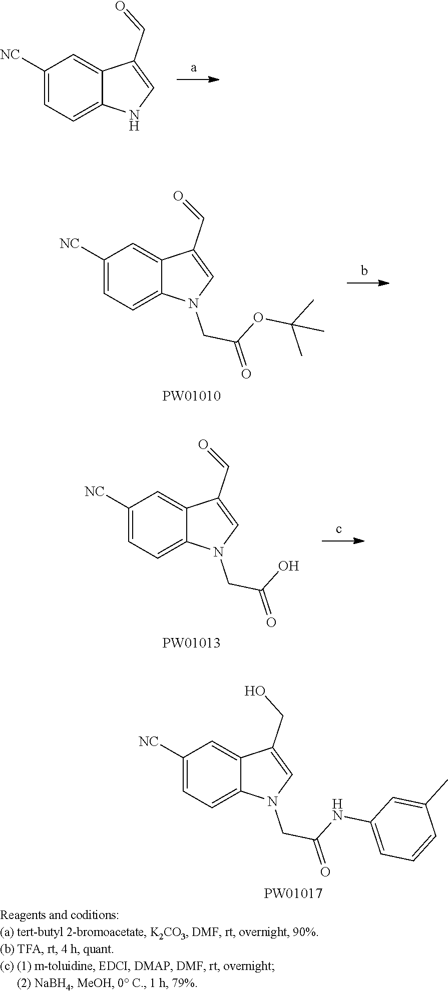

tert-Butyl 2-(5-cyano-3-formyl-1H-indol-1-yl)acetate (PW01010). Following the similar synthetic procedure that used to prepare compound PW0985, compound PW01010 as a white solid (510 mg, 90%). ¹H NMR (300 MHz, Chloroform-d) δ 10.07 (s, 1H), 8.71 (dd, J=1.6, 0.8 Hz, 1H), 7.88 (s, 1H), 7.60 (dd, J=8.6, 1.6 Hz, 1H), 7.38 (dd, J=8.6, 0.7 Hz, 1H), 4.86 (s, 2H), 1.48 (s, 9H).

2-(5-Cyano-3-formyl-1H-indol-1-yl)acetic acid (PW01012). Following the similar synthetic procedure that used to prepare compound PW0985B, compound PW01012 as a white solid (quant.). ¹H NMR (300 MHz, DMSO-d₆) δ 13.18 (s, 1H), 9.96 (s, 1H), 8.51 (s, 1H), 8.44 (dd, J=7.8, 1.6 Hz, 1H), 8.40 (dd, J=4.8, 1.7 Hz, 1H), 7.35 (dd, J=7.8, 4.8 Hz, 1H), 5.17 (s, 2H).

2-(5-Cyano-3-(hydroxymethyl)-1H-indol-1-yl)-N-(m-tolyl)acetamide (PW01017). Following the similar synthetic procedure that used to prepare compound PW0989, compound PW01017 as a white solid (41 mg, 64%). ¹H NMR (300 MHz, DMSO-d₆) δ 10.29 (s, 1H), 8.13 (s, 1H), 7.60 (d, J=8.6 Hz, 1H), 7.49 (d, J=6.7 Hz, 2H), 7.42 (s, 1H), 7.36 (d, J=8.4 Hz, 1H), 7.19 (t, J=7.8 Hz, 1H), 6.88 (d, J=7.5 Hz, 1H), 5.09 (s, 2H), 5.00 (t, J=5.6 Hz, 1H), 4.68 (d, J=5.5 Hz, 2H), 2.26 (s, 3H). ¹³C NMR (75 MHz, DMSO) δ 166.2, 139.1, 139.0, 138.5, 130.6, 129.1, 127.2, 125.3, 124.7, 124.5, 121.1, 120.2, 117.4, 116.8, 111.7, 101.3, 55.4, 49.6, 21.6. HRMS (ESI) calcd for C₁₉H₁₈N₃O₂ 320.1394 (M+H)⁺, found 320.1398.

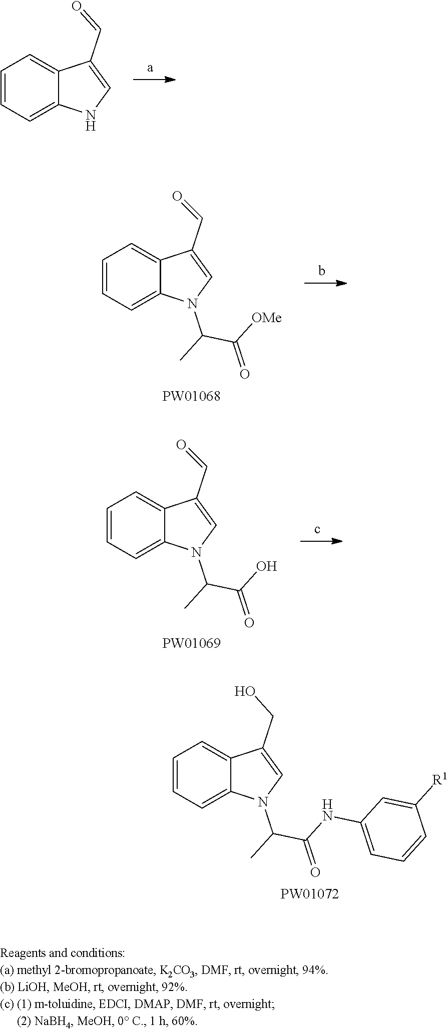

Methyl 2-(3-formyl-1H-indol-1-yl)propanoate (PW01068). Following the similar synthetic procedure that used to prepare compound PW0985, compound PW01068 as a white solid (2.17 g, 94%). ¹H NMR (300 MHz, Chloroform-d) δ 10.08 (s, 1H), 8.42-8.28 (m, 1H), 7.95 (s, 1H), 7.42-7.31 (m, 3H), 5.23 (q, J=7.3 Hz, 1H), 3.77 (s, 3H), 1.92 (d, J=7.3 Hz, 3H).

2-(3-Formyl-1H-indol-1-yl)propanoic acid (PW01069). Following the similar synthetic procedure that used to prepare compound PW0985, compound PW01069 as a white solid (1.0 g, 90%). ¹H NMR (300 MHz, DMSO-d₆) δ 13.23 (s, 1H), 9.95 (s, 1H), 8.47 (s, 1H), 8.26-8.01 (m, 1H), 7.71-7.49 (m, 1H), 7.41-7.16 (m, 2H), 5.50 (q, J=7.2 Hz, 1H), 1.81 (d, J=7.3 Hz, 3H).

2-(3-(Hydroxymethyl)-1H-indol-1-yl)-N-(m-tolyl)propanamide (PW01072). Following the similar synthetic procedure that used to prepare compound PW0989, compound PW01072 as a white solid (35 mg, 60%). ¹H NMR (300 MHz, DMSO-d₆) δ 10.30 (s, 1H), 7.60 (d, J=7.9 Hz, 1H), 7.50 (d, J=8.2 Hz, 1H), 7.40 (dd, J=18.4, 8.9 Hz, 3H), 7.23-6.99 (m, 3H), 6.87 (d, J=7.5 Hz, 1H), 5.30 (q, J=6.6, 5.9 Hz, 1H), 4.82 (t, J=5.5 Hz, 1H), 4.65 (d, J=5.3 Hz, 2H), 2.25 (s, 3H), 1.71 (d, J=7.1 Hz, 3H). ¹³C NMR (75 MHz, DMSO) δ 169.6, 138.9, 138.5, 136.7, 129.1, 127.6, 124.8, 124.6, 121.7, 120.4, 119.7, 119.4, 117.0, 116.4, 110.0, 55.9, 54.5, 21.6, 18.1. HRMS (ESI) calcd for C₁₉H₂₀N₂O₂Na 331.1417 (M+Na)⁺, found 331.1422.

Ethyl 3-(2-(3-(hydroxymethyl)-1H-indol-1-yl)propanamido)benzoate (PW01073). Following the similar synthetic procedure that used to prepare compound PW0989, compound PW01073 as a white solid (38 mg, 52%). ¹H NMR (300 MHz, DMSO-d₆) δ 10.62 (s, 1H), 8.23 (s, 1H), 7.87 (d, J=8.1 Hz, 1H), 7.63 (dd, J=12.9, 7.8 Hz, 2H), 7.47 (q, J=8.2, 7.6 Hz, 3H), 7.14 (t, J=7.7 Hz, 1H), 7.03 (t, J=7.4 Hz, 1H), 5.32 (d, J=7.4 Hz, 1H), 4.82 (s, 1H), 4.65 (d, J=4.2 Hz, 2H), 4.29 (q, J=7.1 Hz, 2H), 1.73 (d, J=6.9 Hz, 3H), 1.30 (t, J=7.1 Hz, 3H). ¹³C NMR (75 MHz, DMSO) δ 170.0, 165.9, 139.4, 136.7, 130.9, 129.8, 127.6, 124.8, 124.7, 124.2, 121.8, 120.2, 119.8, 119.5, 116.5, 110.0, 61.3, 55.9, 54.6, 18.0, 14.6. HRMS (ESI) calcd for C₁₉H₂₀N₂O₂Na, 389.1472 (M+Na)⁺, found 389.1479.

It is to be understood that the foregoing descriptions are exemplary, and thus do not restrict the scope of the invention.

### LIST OF EMBODIMENTS

The following is a non-limiting list of embodiments:
1. A compound of Formula I or a pharmaceutically acceptable salt thereof, wherein:

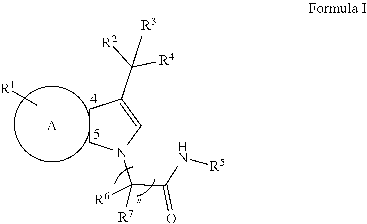

R¹ is H, alkyl, alkoxy, halogen, cyano, amino, hydroxyl, NO₂, CF₃ or —OCF₃;
A is an aryl ring or a heteroaryl ring (e.g., pyridine), wherein ring A is fused to the
C4 and C5 of the 5-membered N-heterocycle ring moiety of Formula I, e.g., as shown the compounds exemplified below;
R² is H, alkyl, alkoxy, halogen, —CO₂R¹⁰, —CO₂Me or hydroxyl;
R³ is H, alkyl, alkoxy, halogen or hydroxyl;
   or R² and R³ together form a 3-6 membered cycloalkyl ring;
R⁴ is H, alkyl, alkoxy, halogen, cyano, hydroxyl or NT¹T²;
T¹ is H, alkyl;
T² is independently chosen from: H, alkyl, cycloalkyl, benzyl, allyl, hydroxyl-alkyl;
   or T¹ and T² together form a 4-12 membered cycloalkyl ring or cycloheteroalkyl ring, wherein the 4-12 membered cycloalkyl ring or cycloheteroalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
n=0, 1, 2, 3, or 4;
R⁵ is H, alkyl, aryl, cycloalkyl (e.g., is a bridged 6-14-membered bicyclic cycloalkyl, with 1-3 carbon length “bridge”); or heteroaryl, wherein each ring is optionally substituted with one or more groups selected independently from: H, alkyl, cycloalkyl, alkenyl, aryl, heteroaryl, benzyl, alkoxy (e.g., OMe, 3-OMe), halogen, cyano (e.g., 3-CN), nitro, amino, amido (e.g., 3-amido hydroxyl, —COOR⁸ (e.g., —COOMe), —CONHR⁹, CHF₂, CF₃ or —OCF₃, wherein alkyl is optionally substituted with one or more substituents chosen from: hydroxyl, cyan, amino, or halogen;
R⁶, and R⁷ are independently chosen from H, alkyl, F, CHF₂, CF₃, etc., or R⁶ and R⁷ taken together form a 3-7 membered ring (e.g., cyclopropane);
R⁸ is independently chosen from: H, alkyl, aryl, cycloalkyl, benzyl; and
R⁹ is independently chosen from: H, alkyl, aryl, cycloalkyl, benzyl, or substituted benzyl.
R¹⁰ is alkyl (e.g., C₁-C₆-alkyl), aryl, or alkylaryl.
2. The compound of embodiment 1, wherein R¹ is H and CN.
3. The compound of embodiment 1, wherein R² is H and —CO₂Me.
4. The compound of embodiment 1, wherein R² and R³ are H.
5. The compound of embodiment 1, wherein R⁴ is OH.
6. The compound of Formula (I) wherein R⁴ is NT¹N².
7. The compound of embodiment 1, wherein n=0, 1, 2, or 3.
8. The compound of embodiment 1, wherein: R¹, R² and R³ are H.
9. The compound of embodiment 1, wherein A is the aryl ring or pyridyl.
10. The compound of embodiment 1, wherein the compound structure is any one of the following:

| Compound | Structure |
|---|---|
| PW0999 | 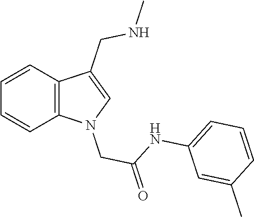 [SMILES: CNCc1cn(CC(=O)Nc2cccc(C)c2)c2ccccc12] |
| PW09100 | 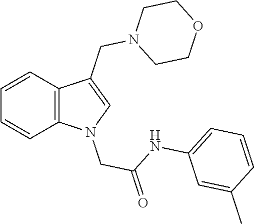 [SMILES: Cc1cccc(NC(=O)Cn2cc(CN3CCOCC3)c3ccccc32)c1] |
| PW01001 | 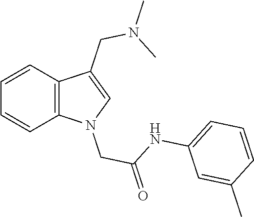 [SMILES: Cc1cccc(NC(=O)Cn2cc(CN(C)C)c3ccccc32)c1] |
| PW01002 | 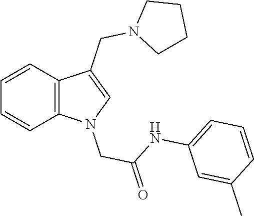 [SMILES: Cc1cccc(NC(=O)Cn2cc(CN3CCCC3)c3ccccc32)c1] |
| PW01003 | 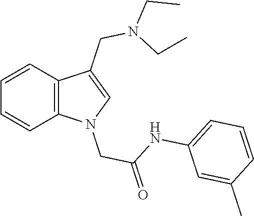 [SMILES: CCN(CC)Cc1cn(CC(=O)Nc2cccc(C)c2)c2ccccc12] |
| PW01004 | 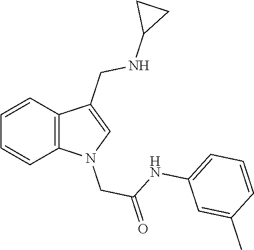 [SMILES: Cc1cccc(NC(=O)Cn2cc(CNC3CC3)c3ccccc32)c1] |
| PW01005 | 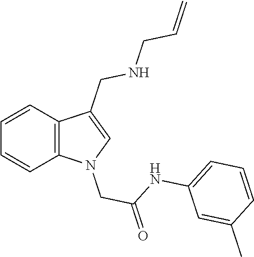 [SMILES: C=CCNCc1cn(CC(=O)Nc2cccc(C)c2)c2ccccc12] |
| PW01006 | 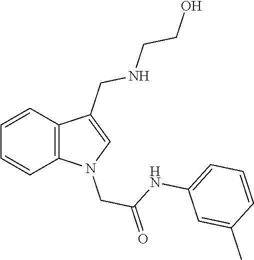 [SMILES: Cc1cccc(NC(=O)Cn2cc(CNCCO)c3ccccc32)c1] |
| PW01007 | 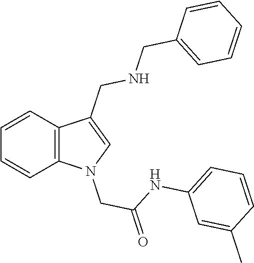 [SMILES: Cc1cccc(NC(=O)Cn2cc(CNCc3ccccc3)c3ccccc32)c1] |
| PW01011 | 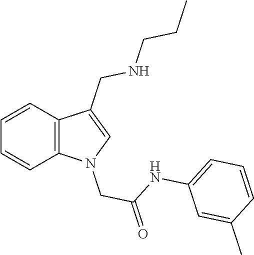 [SMILES: CCCNCc1cn(CC(=O)Nc2cccc(C)c2)c2ccccc12] |
| PW01016 | 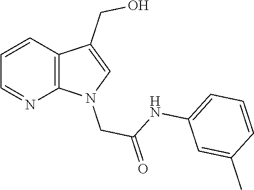 [SMILES: Cc1cccc(NC(=O)Cn2cc(CO)c3cccnc32)c1] |
| PW01017 | 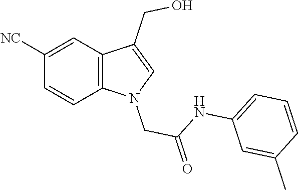 [SMILES: Cc1cccc(NC(=O)Cn2cc(CO)c3cc(C#N)ccc32)c1] |
| PW01020 | 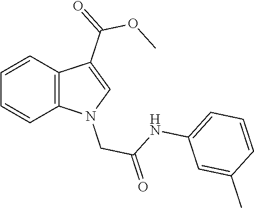 [SMILES: COC(=O)c1cn(CC(=O)Nc2cccc(C)c2)c2ccccc12] |
| PW01022 | 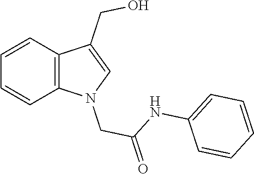 [SMILES: O=C(Cn1cc(CO)c2ccccc21)Nc1ccccc1] |
| PW01023 | 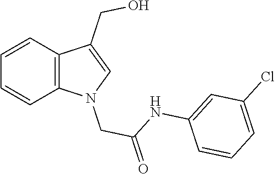 [SMILES: O=C(Cn1cc(CO)c2ccccc21)Nc1cccc(Cl)c1] |
| PW01024 | 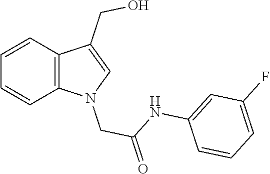 [SMILES: O=C(Cn1cc(CO)c2ccccc21)Nc1cccc(F)c1] |
| PW01025 | 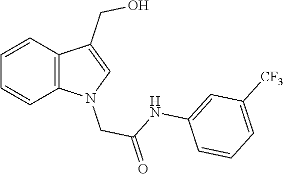 [SMILES: O=C(Cn1cc(CO)c2ccccc21)Nc1cccc(C(F)(F)F)c1] |
| PW01026 | 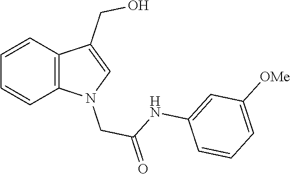 [SMILES: COc1cccc(NC(=O)Cn2cc(CO)c3ccccc32)c1] |
| PW01027 | 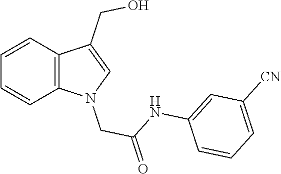 [SMILES: N#Cc1cccc(NC(=O)Cn2cc(CO)c3ccccc32)c1] |
| PW01028 | 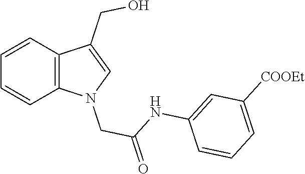 [SMILES: CCOC(=O)c1cccc(NC(=O)Cn2cc(CO)c3ccccc32)c1] |
| PW01029 | 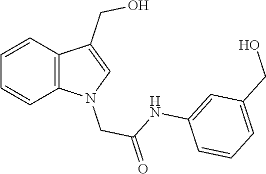 [SMILES: O=C(Cn1cc(CO)c2ccccc21)Nc1cccc(CO)c1] |
| PW01030 | 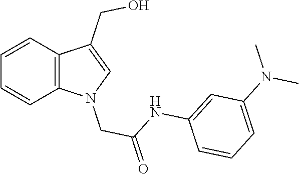 [SMILES: CN(C)c1cccc(NC(=O)Cn2cc(CO)c3ccccc32)c1] |
| PW01031 | 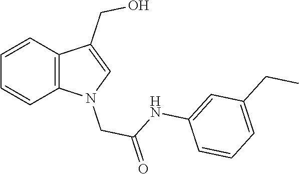 [SMILES: CCc1cccc(NC(=O)Cn2cc(CO)c3ccccc32)c1] |
| PW01032 | 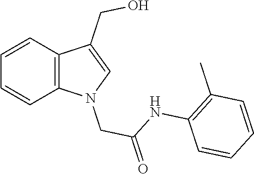 [SMILES: Cc1ccccc1NC(=O)Cn1cc(CO)c2ccccc21] |
| PW01033 | 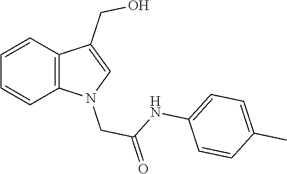 [SMILES: Cc1ccc(NC(=O)Cn2cc(CO)c3ccccc32)cc1] |
| PW01034 | 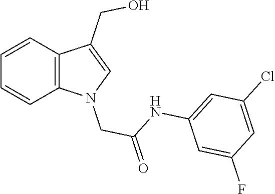 [SMILES: O=C(Cn1cc(CO)c2ccccc21)Nc1cc(F)cc(Cl)c1] |
| PW01035 | 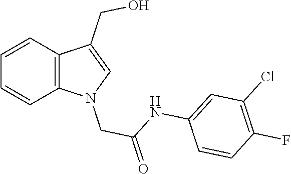 [SMILES: O=C(Cn1cc(CO)c2ccccc21)Nc1ccc(F)c(Cl)c1] |
| PW01036 | 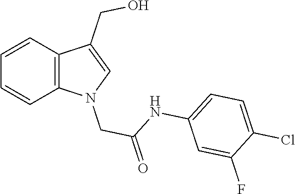 [SMILES: O=C(Cn1cc(CO)c2ccccc21)Nc1ccc(Cl)c(F)c1] |
| PW01037 | 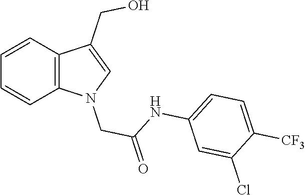 [SMILES: O=C(Cn1cc(CO)c2ccccc21)Nc1ccc(C(F)(F)F)c(Cl)c1] |
| PW01038 | 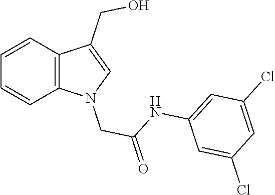 [SMILES: O=C(Cn1cc(CO)c2ccccc21)Nc1cc(Cl)cc(Cl)c1] |
| PW01039 | 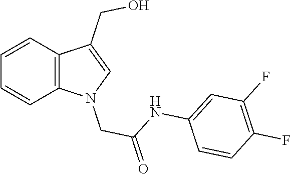 [SMILES: O=C(Cn1cc(CO)c2ccccc21)Nc1ccc(F)c(F)c1] |
| PW01040 | 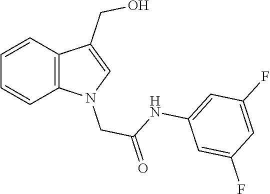 [SMILES: O=C(Cn1cc(CO)c2ccccc21)Nc1cc(F)cc(F)c1] |
| PW01041 | 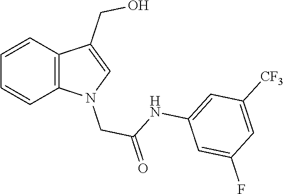 [SMILES: O=C(Cn1cc(CO)c2ccccc21)Nc1cc(F)cc(C(F)(F)F)c1] |
| PW01042 | 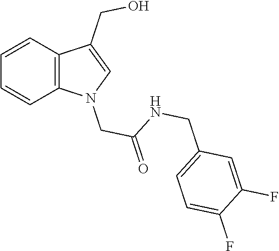 [SMILES: O=C(Cn1cc(CO)c2ccccc21)NCc1ccc(F)c(F)c1] |
| PW01043 | 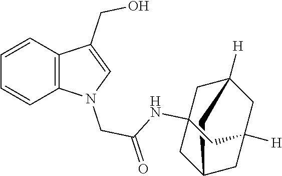 [SMILES: O=C(Cn1cc(CO)c2ccccc21)NC12C[C@H]3C[C@@H](C1)C[C@@H](C2)C3] |
| PW01044 | 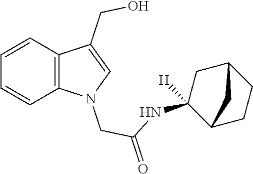 [SMILES: O=C(Cn1cc(CO)c2ccccc21)N[C@H]1C[C@@H]2CC[C@H]1C2] |
| PW01045 | 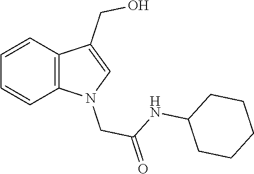 [SMILES: O=C(Cn1cc(CO)c2ccccc21)NC1CCCCC1] |
| PW01046 | 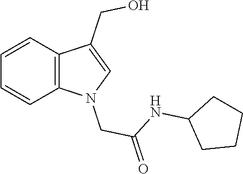 [SMILES: O=C(Cn1cc(CO)c2ccccc21)NC1CCCC1] |
| PW01050 | 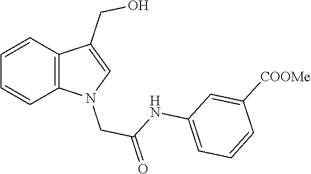 [SMILES: COC(=O)c1cccc(NC(=O)Cn2cc(CO)c3ccccc32)c1] |
| PW01051 | 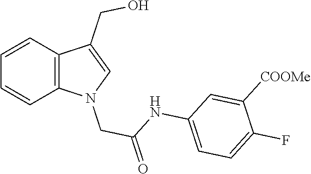 [SMILES: COC(=O)c1cc(NC(=O)Cn2cc(CO)c3ccccc32)ccc1F] |
| PW01053 | 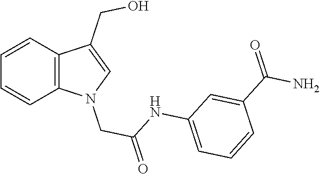 [SMILES: NC(=O)c1cccc(NC(=O)Cn2cc(CO)c3ccccc32)c1] |
| PW01061 | 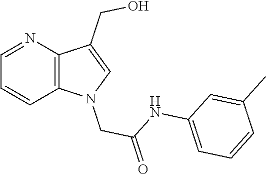 [SMILES: Cc1cccc(NC(=O)Cn2cc(CO)c3ncccc32)c1] |
| PW01062 | 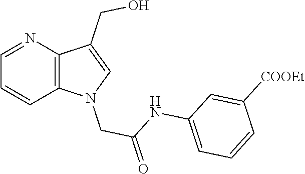 [SMILES: CCOC(=O)c1cccc(NC(=O)Cn2cc(CO)c3ncccc32)c1] |
| PW01072 | 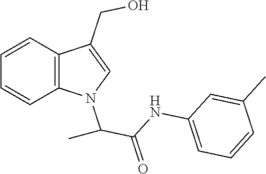 [SMILES: Cc1cccc(NC(=O)C(C)n2cc(CO)c3ccccc32)c1] |
| PW01073 | 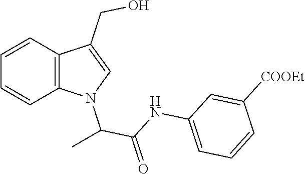 [SMILES: CCOC(=O)c1cccc(NC(=O)C(C)n2cc(CO)c3ccccc32)c1] |
| PW01074 | 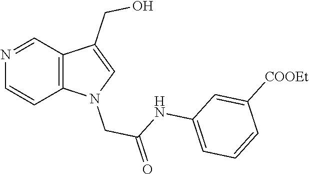 [SMILES: CCOC(=O)c1cccc(NC(=O)Cn2cc(CO)c3cnccc32)c1] |
| PW01076 | 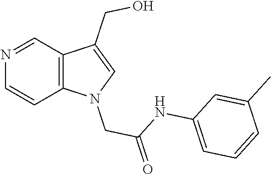 [SMILES: Cc1cccc(NC(=O)Cn2cc(CO)c3cnccc32)c1] |
| PW01083 | 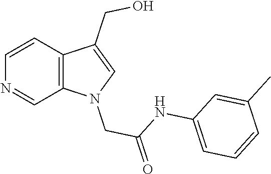 [SMILES: Cc1cccc(NC(=O)Cn2cc(CO)c3ccncc32)c1] |
| PW01084 | 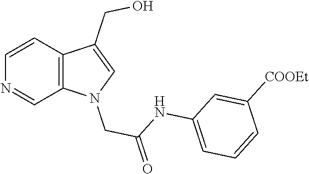 [SMILES: CCOC(=O)c1cccc(NC(=O)Cn2cc(CO)c3ccncc32)c1] |
| JY0137 | 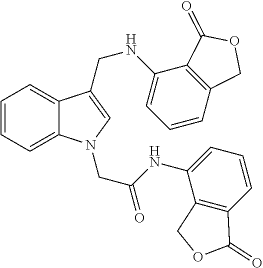 [SMILES: O=C(Cn1cc(CNc2cccc3c2C(=O)OC3)c2ccccc21)Nc1cccc2c1COC2=O] |
| JY0142 | 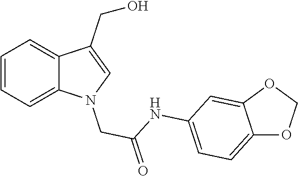 [SMILES: O=C(Cn1cc(CO)c2ccccc21)Nc1ccc2c(c1)OCO2] |
| JY0143 | 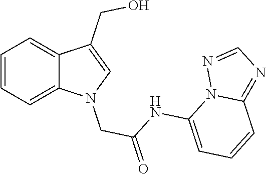 [SMILES: O=C(Cn1cc(CO)c2ccccc21)Nc1cccc2ncnn12] |
| JY0144 | 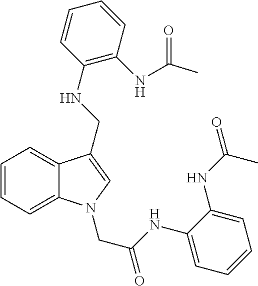 [SMILES: CC(=O)Nc1ccccc1NCc1cn(CC(=O)Nc2ccccc2NC(C)=O)c2ccccc12] |
| JY0145 | 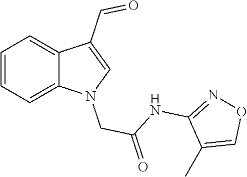 [SMILES: Cc1conc1NC(=O)Cn1cc(C=O)c2ccccc21] |
| JY0147 | 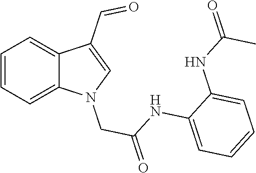 [SMILES: CC(=O)Nc1ccccc1NC(=O)Cn1cc(C=O)c2ccccc21] |
| JY0149 | 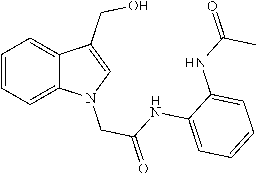 [SMILES: CC(=O)Nc1ccccc1NC(=O)Cn1cc(CO)c2ccccc21] |
| JY0151 | 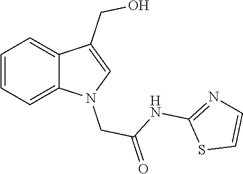 [SMILES: O=C(Cn1cc(CO)c2ccccc21)Nc1nccs1] |
| JY0153 | 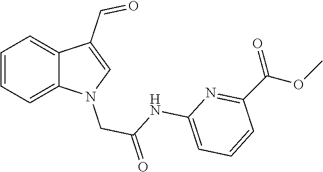 [SMILES: COC(=O)c1cccc(NC(=O)Cn2cc(C=O)c3ccccc32)n1] |
| JY0154 | 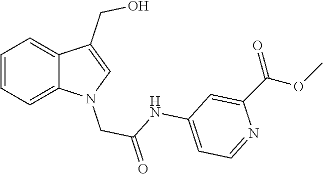 [SMILES: COC(=O)c1cc(NC(=O)Cn2cc(CO)c3ccccc32)ccn1] |
| JY0155 | 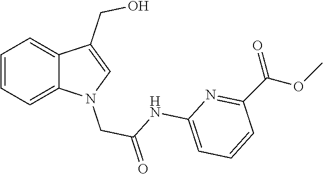 [SMILES: COC(=O)c1cccc(NC(=O)Cn2cc(CO)c3ccccc32)n1] |
| JY0156 | 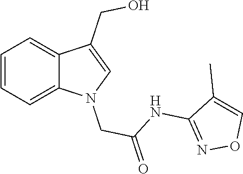 [SMILES: Cc1conc1NC(=O)Cn1cc(CO)c2ccccc21] |
| JY0159 | 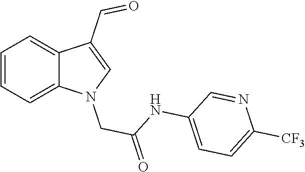 [SMILES: O=Cc1cn(CC(=O)Nc2ccc(C(F)(F)F)nc2)c2ccccc12] |
| JY0160 | 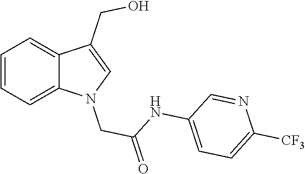 [SMILES: O=C(Cn1cc(CO)c2ccccc21)Nc1ccc(C(F)(F)F)nc1] |
| JY0161 | 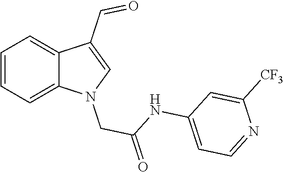 [SMILES: O=Cc1cn(CC(=O)Nc2ccnc(C(F)(F)F)c2)c2ccccc12] |
| JY0162 | 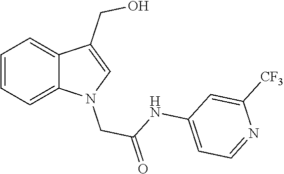 [SMILES: O=C(Cn1cc(CO)c2ccccc21)Nc1ccnc(C(F)(F)F)c1] |
| JY0163 | 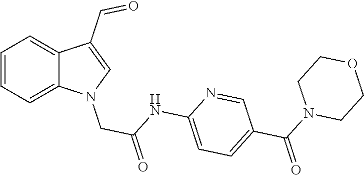 [SMILES: O=Cc1cn(CC(=O)Nc2ccc(C(=O)N3CCOCC3)cn2)c2ccccc12] |
| JY0164 | 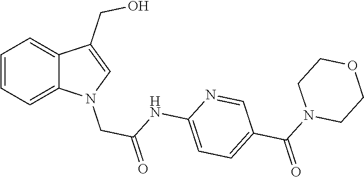 [SMILES: O=C(Cn1cc(CO)c2ccccc21)Nc1ccc(C(=O)N2CCOCC2)cn1] |
| JY0166 | 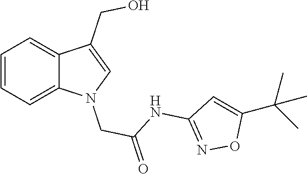 [SMILES: CC(C)(C)c1cc(NC(=O)Cn2cc(CO)c3ccccc32)no1] |
| JY0167 | 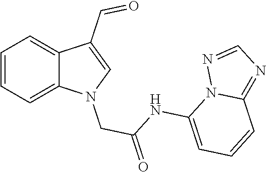 [SMILES: O=Cc1cn(CC(=O)Nc2cccc3ncnn23)c2ccccc12] |
| JY0168 | 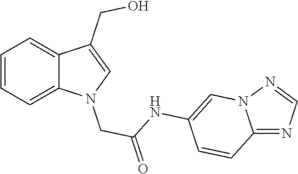 [SMILES: O=C(Cn1cc(CO)c2ccccc21)Nc1ccc2ncnn2c1] |
| JY0169 | 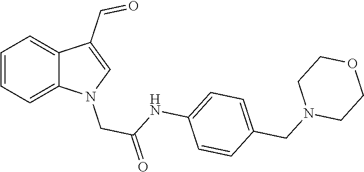 [SMILES: O=Cc1cn(CC(=O)Nc2ccc(CN3CCOCC3)cc2)c2ccccc12] |
| JY0170 | 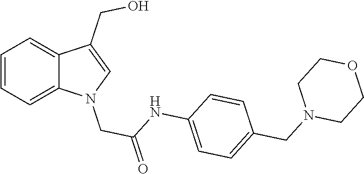 [SMILES: O=C(Cn1cc(CO)c2ccccc21)Nc1ccc(CN2CCOCC2)cc1] |
| JY0172 | 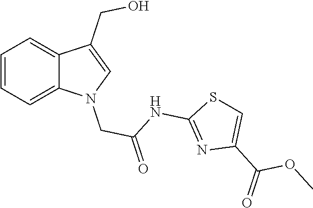 [SMILES: COC(=O)c1csc(NC(=O)Cn2cc(CO)c3ccccc32)n1] |
| JY0186 | 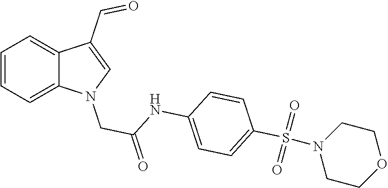 [SMILES: O=Cc1cn(CC(=O)Nc2ccc(S(=O)(=O)N3CCOCC3)cc2)c2ccccc12] |
| JY0187 | 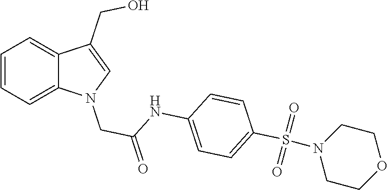 [SMILES: O=C(Cn1cc(CO)c2ccccc21)Nc1ccc(S(=O)(=O)N2CCOCC2)cc1] |
| JY0188 | 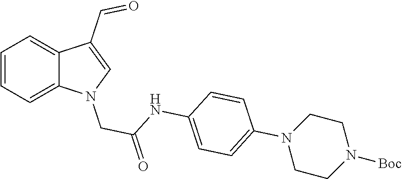 [SMILES: CC(C)(C)OC(=O)N1CCN(c2ccc(NC(=O)Cn3cc(C=O)c4ccccc43)cc2)CC1] |
| JY0189 | 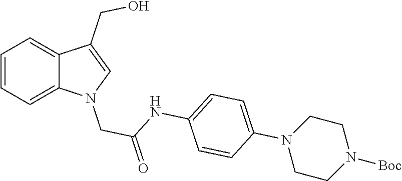 [SMILES: CC(C)(C)OC(=O)N1CCN(c2ccc(NC(=O)Cn3cc(CO)c4ccccc43)cc2)CC1] |
| CZ01 | 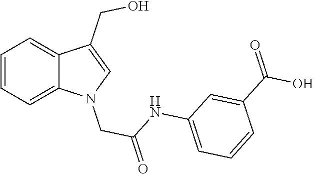 [SMILES: O=C(Cn1cc(CO)c2ccccc21)Nc1cccc(C(=O)O)c1] |
| CZ02 | 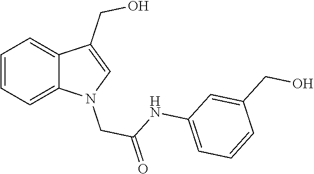 [SMILES: O=C(Cn1cc(CO)c2ccccc21)Nc1cccc(CO)c1] |
| CZ03 | 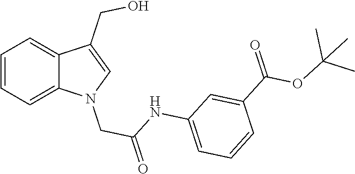 [SMILES: CC(C)(C)OC(=O)c1cccc(NC(=O)Cn2cc(CO)c3ccccc32)c1] |
| CZ04 | 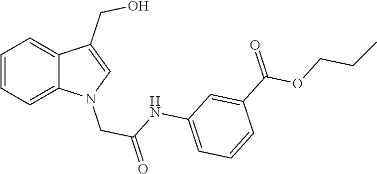 [SMILES: CCCOC(=O)c1cccc(NC(=O)Cn2cc(CO)c3ccccc32)c1] |
| CZ05 | 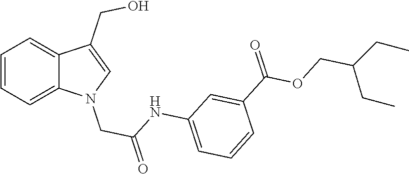 [SMILES: CCC(CC)COC(=O)c1cccc(NC(=O)Cn2cc(CO)c3ccccc32)c1] |
| CZ06 | 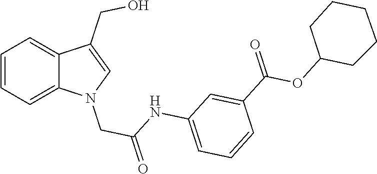 [SMILES: O=C(Cn1cc(CO)c2ccccc21)Nc1cccc(C(=O)OC2CCCCC2)c1] |
| CZ07 | 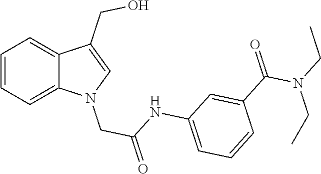 [SMILES: CCN(CC)C(=O)c1cccc(NC(=O)Cn2cc(CO)c3ccccc32)c1] |
| CZ08 | 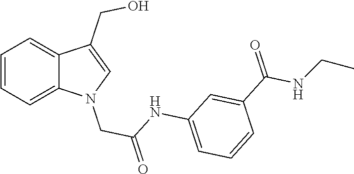 [SMILES: CCNC(=O)c1cccc(NC(=O)Cn2cc(CO)c3ccccc32)c1] |
| CZ09 | 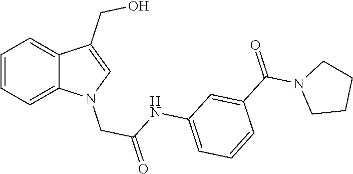 [SMILES: O=C(Cn1cc(CO)c2ccccc21)Nc1cccc(C(=O)N2CCCC2)c1] |
| CZ10 | 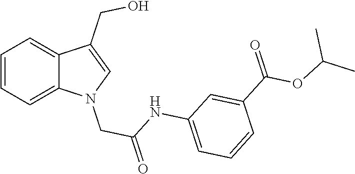 [SMILES: CC(C)OC(=O)c1cccc(NC(=O)Cn2cc(CO)c3ccccc32)c1] |
| CZ11 | 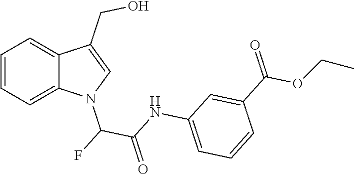 [SMILES: CCOC(=O)c1cccc(NC(=O)C(F)n2cc(CO)c3ccccc32)c1] |
| CZ12 | 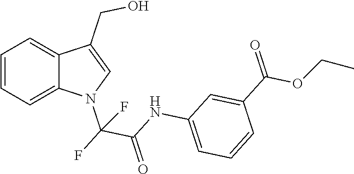 [SMILES: CCOC(=O)c1cccc(NC(=O)C(F)(F)n2cc(CO)c3ccccc32)c1] |
| CZ13 | 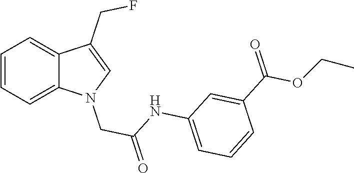 [SMILES: CCOC(=O)c1cccc(NC(=O)Cn2cc(CF)c3ccccc32)c1] |
| CZ14 | 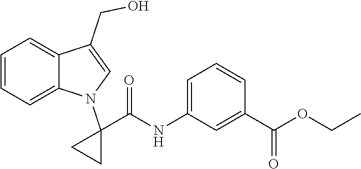 [SMILES: CCOC(=O)c1cccc(NC(=O)C2(n3cc(CO)c4ccccc43)CC2)c1] |
| CZ15 | 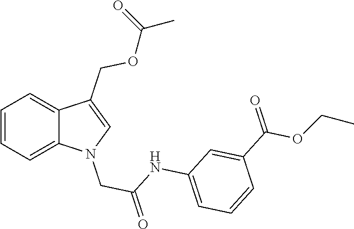 [SMILES: CCOC(=O)c1cccc(NC(=O)Cn2cc(COC(C)=O)c3ccccc32)c1] |
| CZ16 | 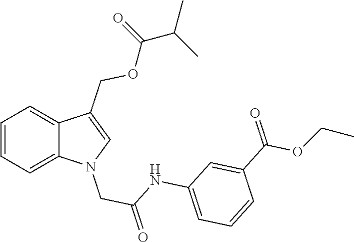 [SMILES: CCOC(=O)c1cccc(NC(=O)Cn2cc(COC(=O)C(C)C)c3ccccc32)c1] |
| CZ17 | 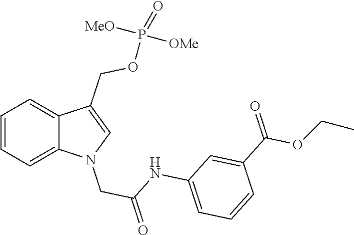 [SMILES: CCOC(=O)c1cccc(NC(=O)Cn2cc(COP(=O)(OC)OC)c3ccccc32)c1] |
| CZ18 | 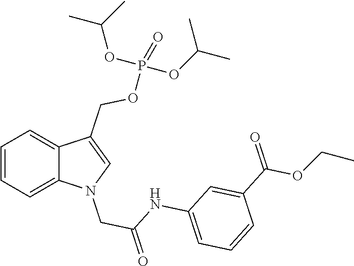 [SMILES: CCOC(=O)c1cccc(NC(=O)Cn2cc(COP(=O)(OC(C)C)OC(C)C)c3ccccc32)c1] |

11. A compound of the Formula (II) or a pharmaceutically acceptable salt thereof, wherein.

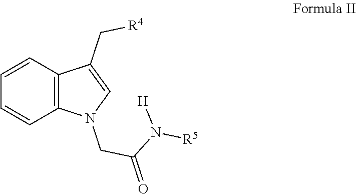

R⁴ is hydroxyl or NT¹T²; wherein T¹ is H or alkyl; and wherein T² is independently chosen from H, alkyl, cycloalkyl, benzyl, allyl, hydroxyl-alkyl;
   or T¹ and T² together form a 4-12 membered cycloalkyl ring or cycloheteroalkyl ring, wherein the 4-12 membered cycloalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
R⁵ is an aryl ring or a cycloalkyl ring, wherein each ring is optionally substituted with one or more groups selected independently from: H, alkyl, cycloalkyl, alkenyl, aryl, heteroaryl, benzyl, alkoxy, halogen, cyano, nitro, amino, hydroxyl, —COOR⁸, —CONR⁹, CHF₂, CF₃ or —OCF₃, wherein alkyl is optionally substituted with one or more chosen substituents chosen from: hydroxyl, cyano, amino, or halogen;
R⁸ is independently chosen from: H, alkyl, aryl, cycloalkyl, or benzyl; and
R⁸ is independently chosen from: H, alkyl, aryl, cycloalkyl, benzyl, or substituted benzyl.
12. The compound of embodiment 11, wherein R⁴ is hydroxyl.
13. The compound of embodiment 11, wherein R⁴ is NT¹T².
14. The compound of embodiment 13, wherein T¹ is H.
15. The compound of embodiment 11, wherein R⁵ is an aryl ring.
16. The compound of embodiment 15, wherein the aryl ring is substituted with one or more substituents selected independently from: H, alkyl, benzyl, alkoxy, halogen, cyano, nitro, amino, hydroxyl, —COOR⁸, —CONR⁹, CHF₂, CF₃ or —OCF₃, wherein alkyl is optionally substituted with one or more chosen substituents chosen from: hydroxyl, cyano, amino, or halogen;
   R⁸ is H, alkyl; and
   R⁹ is independently chosen from: H, alkyl, aryl, cycloalkyl, benzyl, or substituted benzyl.
17. The compound of embodiment 15, wherein the R⁵ aryl ring is substituted with alkyl (e.g., at the 2-, 3-, or 4-position of the aryl ring, such as 3-Me, 3-Et, 2-Me, or 4-Me).
18. A compound of Formula III, or a pharmaceutically acceptable salt thereof, wherein:

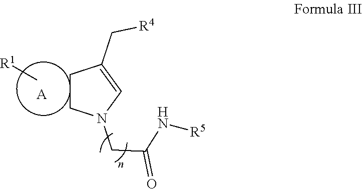

wherein A, n, R¹, R⁴, and R⁵ are as defined for the compound of Formula I.
19. A compound of Formula IV, or a pharmaceutically acceptable salt thereof, wherein:

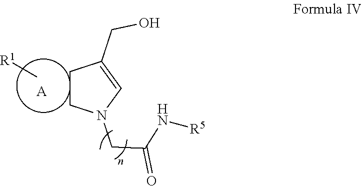

wherein A, n, R¹, and R⁵ are defined as for the compound of Formula I.
20. A compound of Formula V, or a pharmaceutically acceptable salt thereof, wherein:

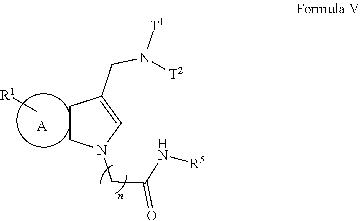

wherein A, n, R¹, R⁵, T¹, and T² are defined as for the compound of Formula I.
21. A compound of Formula VI, or a pharmaceutically acceptable salt thereof, wherein:

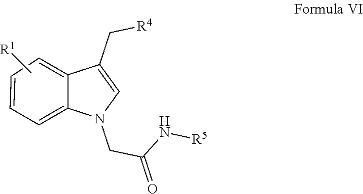

wherein R¹, R⁴, and R⁵ are defined as for the compound of Formula I.
22. A compound of Formula VII, or a pharmaceutically acceptable salt thereof, wherein:

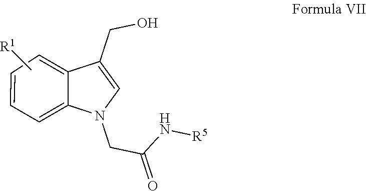

wherein R¹ and R⁵ are defined as for the compound of Formula I.
23. A compound of Formula VIII, or a pharmaceutically acceptable salt thereof, wherein:

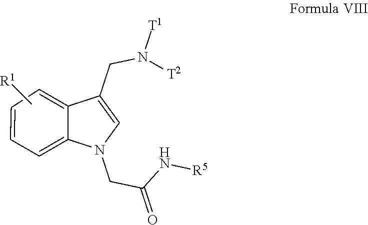

wherein R¹, R¹, T¹, and T² are defined as for the compound of Formula I.
24. A compound of Formula IX, or a pharmaceutically acceptable salt thereof, wherein:

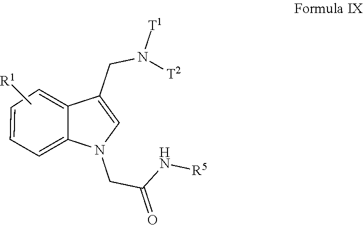

wherein R¹, R⁵, T¹, and T² are defined as for the compound of Formula I.
25. A compound of Formula X, or a pharmaceutically acceptable salt thereof, wherein.

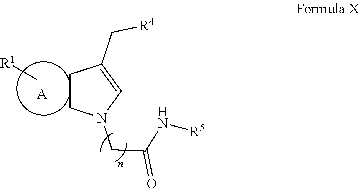

wherein A is a heteroaryl ring, n, R¹, R⁴ and R⁵ are defined as for the compound of Formula I.
26. A method of treating a disease or condition in a patient comprising administering to the patient a therapeutically effective amount of a compound of any of the preceding embodiments or a pharmaceutically acceptable salt thereof.
27. The method of embodiment 26, wherein treatment of the disease or condition involves modulating a FGF14:NAV1.6 channel complex protein-protein interaction.
28. The method of embodiment 26, wherein said compound modulates voltage-gated Na+ (Nav) channels and their isoforms (such as Nav1.6).
29. The method of embodiment 26, wherein said compound modulates voltage-gated sodium channels and their accessory regulator proteins complex protein-protein interactions.
30. The method of embodiment 26, wherein said compound modulates voltage-gated sodium channel accessory regulator proteins (such as intracellular fibroblast growth factors (e.g., FGF14, FGF13, FGF12 and FGF11)).
31. The method of embodiment 26, said compound modulates FGF14:Nav1.6 channel complex formation and increases Nav1.6 channel availability (and medium spiny neuron (MSN) firing).
32. The method of embodiment 26, said compound modulates reward-related behaviors within the mesocorticolimbic circuit.
33. The method of embodiment 26, wherein the disease or condition is a central nervous system chosen from any of brain disorders, neurological disorders, psychiatric disorders, substance abuse disorders, neuroinflammation, or pain.
34. The method of embodiment 26, wherein the compound of Formula (I) is compound PW0989.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
1 Cuthbert, B. N. The role of RDoC in future classification of mental disorders Dialogues in clinical neuroscience 22, 81-85, doi:10.31887/DCNS.2020.22.1/bcuthbert (2020). PubMed Central ID:PMCPMC7365298I.
2 Emmett, M. R., Kroes, R. A., Moskal, J. R., Conrad, C. A., Priebe, W., Laezza, F., Meyer-Baese, A. & Nilsson, C. L. Integrative biological analysis for neuropsychopharmacology. Neuropsychopharmacology 39, 5-23, doi:10.1038/npp.2013.156 (2014). PubMed Central ID:PMC3857644I.
3 Wildburger, N. C., Ali, S. R., Hsu, W. C. J., Shavkunov, A. S., Nenov, M. N., Lichti, C. F., LeDuc, R. D., Mostovenko, E., Panova-Elektronova, N. I., Emmett, M. R., Nilsson, C. L. & Laezza, F. Quantitative Proteomics Reveals Protein-Protein Interactions with Fibroblast Growth Factor 12 as a Component of the Voltage-Gated Sodium Channel 1.2 (Nav1.2) Macromolecular Complex in Mammalian Brain. Mol Cell Proteomics 14, 1288-1300, doi:10.1074/mcp.M114.040055 (2015). PubMed Central ID:PMCPMC4424400I.
4 Hsu, W. C., Nilsson, C. L. & Laezza, F. Role of the axonal initial segment in psychiatric disorders: function, dysfunction, and intervention. Frontiers in psychiatry 5, 109, doi:10.3389/fpsyt.2014.00109 (2014). PubMed Central ID:PMC4139700I.
5 Cui, Y., Yang, Y., Ni, Z., Dong, Y., Cai, G., Foncelle, A., Ma, S., Sang, K., Tang, S., Li, Y., Shen, Y., Berry, H., Wu, S. & Hu, H. Astroglial Kir4.1 in the lateral habenula drives neuronal bursts in depression. Nature 554, 323-327, doi:10.1038/nature25752 (2018)
6 Li, S. X., Han, Y., Xu, L. Z., Yuan, K., Zhang, R. X., Sun, C. Y., Xu, D. F., Yuan, M., Deng, J. H., Meng, S. Q., Gao, X. J., Wen, Q., Liu, L. J., Zhu, W. L., Xue, Y. X., Zhao, M., Shi, J. & Lu, L. Uncoupling DAPK1 from NMDA receptor GluN2B subunit exerts rapid antidepressant-like effects. Mol Psychiatry 23, 597-608, doi:10.1038/mp.2017.85 (2018). PubMed Central ID:PMCPMC5822462I.
7 Shao, L. X., Jiang, Q., Liu, X. X., Gong, D. M., Yin, Y. X., Wu, G., Sun, N. H., Wang, C. K., Chen, Q. Z., Yu, C., Shi, W. X., Fan, H. Y., Fukunaga, K., Chen, Z., Lu, Y. M. & Han, F. Functional coupling of Tmem74 and HCN1 channels regulates anxiety-like behavior in BLA neurons. Mol Psychiatry 24, 1461-1477, doi:10.1038/s41380-019-0402-8 (2019)
8 Arkin, M. R. & Wells, J. A. Small-molecule inhibitors of protein-protein interactions: progressing towards the dream. Nat Rev Drug Discov 3, 301-317 (2004)
9 Kenny, P. J., Hoyer, D. & Koob, G. F. Animal Models of Addiction and Neuropsychiatric Disorders and Their Role in Drug Discovery: Honoring the Legacy of Athina Markou. Biol Psychiatry 83, 940-946, doi:10.1016/j.biopsych.2018.02.009 (2018)
10 Salgado, S. & Kaplitt, M. G. The Nucleus Accumbens: A Comprehensive Review. Stereotact Funct Neurosurg 93, 75-93, doi:10.1159/000368279 (2015)
11 Medeiros, G. C., Rush, A. J., Jha, M., Carmody, T., Furman, J. L., Czysz, A. H., Trombello, J. M., Cooper, C. M. & Trivedi, M. H. Positive and negative valence systems in major depression have distinct clinical features, response to antidepressants, and relationships with immunomarkers. Depress Anxiety, doi:10.1002/da.23006 (2020)
12 Da Silva, S., Apatsidou, A., Saperia, S., Siddiqui, I., Jeffay, E., Voineskos, A. N., Daskalakis, Z. J., Remington, G., Zakzanis, K. K. & Foussias, G. An Examination of the Multi-Faceted Motivation System in Healthy Young Adults. Frontiers in psychiatry 9, 191, doi:10.3389/fpsyt.2018.00191 (2018). PubMed Central ID:PMCPMC5958204I.
13 Clery-Melin, M. L., Jollant, F. & Gorwood, P. Reward systems and cognitions in Major Depressive Disorder. CNS Spectr 24, 64-77, doi:10.1017/s1092852918001335 (2019)
14 Gondre-Lewis, M. C., Bassey, R. & Blum, K. Pre-clinical models of reward deficiency syndrome: A behavioral octopus. Neurosci Biobehav Rev 115, 164-188, doi:10.1016/j.neubiorev.2020.04.021 (2020)
15 Russo, S. J., Murrough, J. W., Han, M. H., Charney, D. S. & Nestler, E. J. Neurobiology of resilience. Nat Neurosci 15, 1475-1484, doi:10.1038/nn.3234 (2012). PubMed Central ID:PMCPMC3580862I.
16 Russo, S. J. & Nestler, E. J. The brain reward circuitry in mood disorders. Nat Rev Neurosci 14, 609-625, doi:10.1038/nrn3381 (2013). PubMed Central ID:PMC3867253I.
17 Tsutsui-Kimura, I., Natsubori, A., Mori, M., Kobayashi, K., Drew, M. R., de Kerchove d'Exaerde, A., Mimura, M. & Tanaka, K. F. Distinct Roles of Ventromedial versus Ventrolateral Striatal Medium Spiny Neurons in Reward-Oriented Behavior. Curr Biol 27, 3042-3048.e3044, doi:10.1016/j.cub.2017.08.061 (2017)
18 Riga, D., Theijs, J. T., De Vries, T. J., Smit, A. B. & Spijker, S. Social defeat-induced anhedonia: effects on operant sucrose-seeking behavior. Front Behav Neurosci 9, 195, doi:10.3389/fnbeh.2015.00195 (2015). PubMed Central ID:PMCPMC4528167I.
19 Natsubori, A., Tsutsui-Kimura, I., Nishida, H., Bouchekioua, Y., Sekiya, H., Uchigashima, M., Watanabe, M., de Kerchove d'Exaerde, A., Mimura, M., Takata, N. & Tanaka, K. F. Ventrolateral Striatal Medium Spiny Neurons Positively Regulate Food-Incentive, Goal-Directed Behavior Independently of D1 and D2 Selectivity. J Neurosci 37, 2723-2733, doi:10.1523/jneurosci.3377-16.2017 (2017). PubMed Central ID:PMCPMC6596637I.
20 Ali, S. R., Liu, Z., Nenov, M. N., Folorunso, O., Singh, A., Scala, F., Chen, H., James, T. F., Alshammari, M., Panova-Elektronova, N. I., White, M. A., Zhou, J. & Laezza, F. Functional Modulation of Voltage-Gated Sodium Channels by a FGF14-Based Peptidomimetic. ACS Chem Neurosci, doi:10.1021/acschemneuro.7b00399 (2018). PubMed Central ID:PMCPMC6033619I.
21 Ali, S. R., Singh, A. K. & Laezza, F. Identification of Amino Acid Residues in the Fibroblast Growth Factor 14 (FGF14) Required For Structure-Function interactions with the Voltage-Gated Sodium Channel Nav1.6. J Biol Chem, doi:10.1074/jbc.M115.703868 (2016). PubMed Central ID:PMCPMC4900273I.
22 Alshammari, M. A., Alshammari, T. K., Nenov, M. N., Scala, F. & Laezza, F. Fibroblast Growth Factor 14 Modulates the Neurogenesis of Granule Neurons in the Adult Dentate Gyrus. Molecular neurobiology, doi:10.1007/s12035-015-9568-5 (2015)
23 Alshammari, T. K., Alshammari, M. A., Nenov, M. N., Hoxha, E., Cambiaghi, M., Marcinno, A., James, T. F., Singh, P., Labate, D., Li, J., Meltzer, H. Y., Sacchetti, B., Tempia, F. & Laezza, F. Genetic deletion of fibroblast growth factor 14 recapitulates phenotypic alterations underlying cognitive impairment associated with schizophrenia. Translational psychiatry 6, e806-e806, doi:10.1038/tp.2016.66 (2016). PubMed Central ID:PMCPMC5070049I.
24 Shavkunov, A. S., Wildburger, N. C., Nenov, M. N., James, T. F., Buzhdygan, T. P., Panova-Elektronova, N. I., Green, T. A., Veselenak, R. L., Bourne, N. & Laezza, F. The Fibroblast Growth Factor 14:Voltage-gated Sodium Channel Complex Is a New Target of Glycogen Synthase Kinase 3 (GSK3). J Biol Chem 288, 19370-19385PMC3707642 (2013). PubMed Central ID:PMCPMC3707642I.
25 Wadsworth, P. A., Singh, A. K., Nguyen, N., Dvorak, N. M., Tapia, C. M., Russell, W. K., Stephan, C. & Laezza, F. JAK2 regulates Nav1.6 channel function via FGF14(Y158) phosphorylation. Biochim Biophys Acta Mol Cell Res 1867, 118786, doi:10.1016/j.bbamcr.2020.118786 (2020). PubMed Central ID:PMCIn progressI.
26 Singh, A. K., Wadsworth, P. A., Tapia, C. M., Aceto, G., Ali, S. R., Chen, H., D'Ascenzo, M., Zhou, J. & Laezza, F. Mapping of the FGF14:Nav.6 complex interface reveals FLPK as a functionally active peptide modulating excitability. Physiol Rep 8, e14505, doi:10.14814/phy2.14505 (2020). PubMed Central ID:PMCPMC7363588I.
27 Tempia, F., Hoxha, E., Negro, G., Alshammari, M. A., Alshammari, T. K., Panova-Elektronova, N. & Laezza, F. Parallel fiber to Purkinje cell synaptic impairment in a mouse model of spinocerebellar ataxia type 27. Frontiers in Cellular Neuroscience 9, doi:Artn 205
10.3389/Fncel.2015.00205 (2015)
28 Di Re, J., Wadsworth, P. A. & Laezza, F. Intracellular Fibroblast Growth Factor 14: Emerging Risk Factor for Brain Disorders. Front Cell Neurosci 11, 103, doi:10.3389/fncel.2017.00103 (2017). PubMed Central ID:PMCPMC5396478I.
29 Hsu, W. J., Wildburger, N. C., Haidacher, S. J., Nenov, M. N., Folorunso, O., Singh, A. K., Chesson, B. C., Franklin, W. F., Cortez, I., Sadygov, R. G., Dineley, K. T., Rudra, J. S., Taglialatela, G., Lichti, C. F., Denner, L. & Laezza, F. PPARgamma agonists rescue increased phosphorylation of FGF14 at S226 in the Tg2576 mouse model of Alzheimer's disease. Exp Neurol 295, 1-17, doi:10.1016/j.expneurol.2017.05.005 (2017). PubMed Central ID:PMCPMC5762202I.
30 Sowers, M. L., Re, J. D., Wadsworth, P. A., Shavkunov, A. S., Lichti, C., Zhang, K. & Laezza, F. Sex-Specific Proteomic Changes Induced by Genetic Deletion of Fibroblast Growth Factor 14 (FGF14), a Regulator of Neuronal Ion Channels. Proteomes 7, doi:10.3390/proteomes7010005 (2019). PubMed Central ID:PMCPMC6473632I.
31 Hsu, W. J., Scala, F., Nenov, M. N., Wildburger, N. C., Elferink, H., Singh, A. K., Chesson, C. B., Buzhdygan, T., Sohail, M., Shavkunov, A. S., Panova, N. I., Nilsson, C. L., Rudra, J. S., Lichti, C. F. & Laezza, F. CK2 activity is required for the interaction of FGF14 with voltage-gated sodium channels and neuronal excitability. FASEB journal: official publication of the Federation of American Societies for Experimental Biology, doi:10.1096/fj.201500161 (2016). PubMed Central ID:PMCPMC4871802I.
32 Hsu, W. C., Nenov, M. N., Shavkunov, A., Panova, N., Zhan, M. & Laezza, F. Identifying a kinase network regulating FGF14:Nav1.6 complex assembly using split-luciferase complementation. PloS one 10, e0117246, doi:10.1371/journal.pone.0117246 (2015). PubMed Central ID:PMC4319734I.
33 Dvorak, N. M., Wadsworth, P. A., Wang, P., Chen, H., Zhou, J. & Laezza, F. Bidirectional Modulation of the Voltage-Gated Sodium (Nav1.6) Channel by Rationally Designed Peptidomimetics. Molecules 25, doi:10.3390/molecules25153365 (2020). PubMed Central ID:PMCIn progressI.
34 Laezza, F., Gerber, B. R., Lou, J. Y., Kozel, M. A., Hartman, H., Craig, A. M., Ornitz, D. M. & Nerbonne, J. M. The FGF14(F145S) mutation disrupts the interaction of FGF14 with voltage-gated Na+ channels and impairs neuronal excitability. J Neurosci 27, 12033-12044 (2007)
35 Wozniak, D. F., Xiao, M., Xu, L., Yamada, K. A. & Ornitz, D. M. Impaired Spatial Learning and Defective Theta Burst Induced LTP in Mice Lacking Fibroblast Growth Factor 14. Neurobiol Dis 1, 14-26 (2007)
36 Shakkottai, V. G., Xiao, M., Xu, L., Wong, M., Nerbonne, J. M., Ornitz, D. M. & Yamada, K. A. FGF14 regulates the intrinsic excitability of cerebellar Purkinje neurons. Neurobiol Dis 33, 81-88PMC2652849 (2009). PubMed Central ID:PMCPMC2652849I.
37 Goldfarb, M., Schoorlemmer, J., Williams, A., Diwakar, S., Wang, Q., Huang, X., Giza, J., Tchetchik, D., Kelley, K., Vega, A., Matthews, G., Rossi, P., Ornitz, D. M. & D'Angelo, E. Fibroblast growth factor homologous factors control neuronal excitability through modulation of voltage-gated sodium channels. Neuron 55, 449-463 (2007)
38 Dover, K., Solinas, S., D'Angelo, E. & Goldfarb, M. Long-term inactivation particle for voltage-gated sodium channels. J Physiol 588, 3695-3711, doi:10.1113/jphysiol.2010.192559 (2010). PubMed Central ID:PMCPMC2998221I.
39 White, H. V., Brown, S. T., Bozza, T. C. & Raman, I. M. Effects of FGF14 and Na(V)β4 deletion on transient and resurgent Na current in cerebellar Purkinje neurons. J Gen Physiol 151, 1300-1318, doi:10.1085/jgp.201912390 (2019). PubMed Central ID:PMCPMC6829560I.
40 Brusse, E., de Koning, I., Maat-Kievit, A., Oostra, B. A., Heutink, P. & van Swieten, J. C. Spinocerebellar ataxia associated with a mutation in the fibroblast growth factor 14 gene (SCA27): A new phenotype. Mov Disord 21, 396-401 (2006)
41 Van Swieten, J. C., Brusse, E., De Graaf, B. M., Krieger, E., Van De Graaf, R., De Koning, I., Maat-Kievit, A., Leegwater, P., Dooijes, D., Oostra, B. A. & Heutink, P. A mutation in the fibroblast growth factor 14 gene is associated with autosomal dominant cerebellar ataxia. Am J Hum Genet 72, 191-199 (2003)
42 Verbeek, E. C., Bakker, I. M., Bevova, M. R., Bochdanovits, Z., Rizzu, P., Sondervan, D., Willemsen, G., de Geus, E. J., Smit, J. H., Penninx, B. W., Boomsma, D. I., Hoogendijk, W. J. & Heutink, P. A fine-mapping study of 7 top scoring genes from a GWAS for major depressive disorder. PloS one 7, e37384PMC3359349 (2012). PubMed Central ID:PMCPMC3359349I.
43 Vadasz, C., Smiley, J. F., Figarsky, K., Saito, M., Toth, R., Gyetvai, B. M., Oros, M., Kovacs, K. K., Mohan, P. & Wang, R. Mesencephalic dopamine neuron number and tyrosine hydroxylase content: Genetic control and candidate genes. Neuroscience 149, 561-572 (2007)
44 Paucar, M., Lundin, J., Alshammari, T., Bergendal, A., Lindefeldt, M., Alshammari, M., Solders, G., Di Re, J., Savitcheva, I., Granberg, T., Laezza, F., Iwarsson, E. & Svenningsson, P. Broader phenotypic traits and widespread brain hypometabolism in spinocerebellar ataxia 27. J Intern Med 288, 103-115, doi:10.1111/joim.13052 (2020). PubMed Central ID:PMCIn progressI.
45 Scala, F., Nenov, M. N., Crofton, E. J., Singh, A. K., Folorunso, O., Zhang, Y., Chesson, B. C., Wildburger, N. C., James, T. F., Alshammari, M. A., Alshammari, T. K., Elfrink, H., Grassi, C., Kasper, J. M., Smith, A. E., Hommel, J. D., Lichti, C. F., Rudra, J. S., D'Ascenzo, M., Green, T. A. & Laezza, F. Environmental Enrichment and Social Isolation Mediate Neuroplasticity of Medium Spiny Neurons through the GSK3 Pathway. Cell Rep 23, 555-567, doi:10.1016/j.celrep.2018.03.062 (2018). PubMed Central ID:PMCPMC6150488I.
46 Crofton, E. J., Nenov, M. N., Zhang, Y., Scala, F., Page, S. A., McCue, D. L., Li, D., Hommel, J. D., Laezza, F. & Green, T. A. Glycogen synthase kinase 3 beta alters anxiety-, depression-, and addiction-related behaviors and neuronal activity in the nucleus accumbens shell. Neuropharmacology 117, 49-60, doi:10.1016/j.neuropharm.2017.01.020 (2017). PubMed Central ID:PMCPMC5386787I.
47 Li, X. & Jope, R. S. Is glycogen synthase kinase-3 a central modulator in mood regulation?Neuropsychopharmacology 35, 2143-2154PMC3055312 (2010). PubMed Central ID:PMCPMC3055312I.
48 Weng, Y. T., Chien, T., Kuan, II & Chern, Y. The TRAX, DISC1, and GSK3 complex in mental disorders and therapeutic interventions. J Biomed Sci 25, 71, doi:10.1186/s12929-018-0473-x (2018). PubMed Central ID:PMCPMC6171312I.
49 Zanos, P., Thompson, S. M., Duman, R. S., Zarate, C. A., Jr. & Gould, T. D. Convergent Mechanisms Underlying Rapid Antidepressant Action. CNS Drugs 32, 197-227, doi:10.1007/s40263-018-0492-x (2018). PubMed Central ID:PMCPMC6005380I.
50 Ali, S., Shavkunov, A., Panova, N., Stoilova-McPhie, S. & Laezza, F. Modulation of the FGF14:FGF14 homodimer interaction through short peptide fragments. CNS & neurological disorders drug targets 13, 1559-1570PMC4809639 (2014). PubMed Central ID:PMCPMC4809639I.
51 Goetz, R., Dover, K., Laezza, F., Shtraizent, N., Huang, X., Tchetchik, D., Eliseenkova, A. V., Xu, C. F., Neubert, T. A., Ornitz, D. M., Goldfarb, M. & Mohammadi, M. Crystal structure of a fibroblast growth factor homologous factor (FHF) defines a conserved surface on FHFs for binding and modulation of voltage-gated sodium channels. J Biol Chem 284, 17883-17896PMC2719427 (2009). PubMed Central ID:PMCPMC2719427I.
52 Wadsworth, P. A., Folorunso, O., Nguyen, N., Singh, A. K., D'Amico, D., Powell, R. T., Brunell, D., Allen, J., Stephan, C. & Laezza, F. High-throughput screening against protein:protein interaction interfaces reveals anti-cancer therapeutics as potent modulators of the voltage-gated Na(+) channel complex. Sci Rep 9, 16890, doi:10.1038/s41598-019-53110-8 (2019). PubMed Central ID:PMCPMC6858373I.
53 Liu, Z., Wadsworth, P., Singh, A. K., Chen, H., Wang, P., Folorunso, O., Scaduto, P., Ali, S. R., Laezza, F. & Zhou, J. Identification of peptidomimetics as novel chemical probes modulating fibroblast growth factor 14 (FGF14) and voltage-gated sodium channel 1.6 (Nav1.6) protein-protein interactions. Bioorg Med Chem Lett 29, 413-419, doi:10.1016/j.bmcl.2018.12.031 (2019). PubMed Central ID:PMCPMC6748043I.
54 Yan, H., Pablo, J. L., Wang, C. & Pitt, G. S. FGF14 modulates resurgent sodium current in mouse cerebellar Purkinje neurons. eLife 3, e04193-e04193, doi:10.7554/eLife.04193 (2014)
55 Gribkoff, V. K. & Kaczmarek, L. K. The need for new approaches in CNS drug discovery: Why drugs have failed, and what can be done to improve outcomes. Neuropharmacology 120, 11-19, doi:10.1016/j.neuropharm.2016.03.021 (2017). PubMed Central ID:PMCPMC5820030I.
56 Luttrell, L. M., Maudsley, S. & Bohn, L. M. Fulfilling the Promise of “Biased” G Protein-Coupled Receptor Agonism. Molecular pharmacology 88, 579-588, doi:10.1124/mol.115.099630 (2015). PubMed Central ID:PMCPMC4551052I.
57 Changeux, J. P. & Christopoulos, A. Allosteric Modulation as a Unifying Mechanism for Receptor Function and Regulation. Cell 166, 1084-1102, doi:10.1016/j.cell.2016.08.015 (2016)
58 Thal, D. M., Glukhova, A., Sexton, P. M. & Christopoulos, A. Structural insights into G-protein-coupled receptor allostery. Nature 559, 45-53, doi:10.1038/s41586-018-0259-z (2018)
59 Wootten, D., Christopoulos, A., Marti-Solano, M., Babu, M. M. & Sexton, P. M. Mechanisms of signalling and biased agonism in G protein-coupled receptors. Nat Rev Mol Cell Biol 19, 638-653, doi:10.1038/s41580-018-0049-3 (2018)
60 Dean, B., Moller, H. J., Svensson, T. H., Geyer, M. A., Rujescu, D., Scarr, E. & Millan, M. J. Problems and solutions to filling the drying drug pipeline for psychiatric disorders: a report from the inaugural 2012 CINP Think Tank. Int J Neuropsychopharmacol, 1-12 (2013)
61 Scarr, E., Millan, M. J., Bahn, S., Bertolino, A., Turck, C. W., Kapur, S., Möller, H. J. & Dean, B. Biomarkers for Psychiatry: The Journey from Fantasy to Fact, a Report of the 2013 CINP Think Tank. Int J Neuropsychopharmacol 18, pyv042, doi:10.1093/ijnp/pyv042 (2015). PubMed Central ID:PMCPMC4648162I.
62 Hurko, O. & Ryan, J. L. Translational research in central nervous system drug discovery. NeuroRx 2, 671-682, doi:10.1602/neurorx.2.4.671 (2005). PubMed Central ID:PMCPMC1201324I.
63 Stoilova-McPhie, S., Ali, S. & Laezza, F. Protein-Protein Interactions as New Targets for Ion Channel Drug Discovery. Austin journal of pharmacology and therapeutics 1PMC4255474 (2013). PubMed Central ID:PMCPMC4255474I.
64 Shavkunov, A., Panova, N., Prasai, A., Veselenak, R., Bourne, N., Stoilova-McPhie, S. & Laezza, F. Bioluminescence methodology for the detection of protein-protein interactions within the voltage-gated sodium channel macromolecular complex. Assay Drug Dev Technol 10, 148-160PMC3362325 (2012). PubMed Central ID:PMCPMC3362325I.
65 Shavkunov, A. S., Ali, S. R., Panova-Elektronova, N. I. & Laezza, F. Split-Luciferase Complementation Assay to Detect Channel-Protein Interactions in Live Cells. Protein-Protein Interactions: Methods and Applications, 497-514 (2015)
66 Wang, P., Wadsworth, P. A., Dvorak, N. M., Singh, A. K., Chen, H., Liu, Z., Zhou, R., Holthauzen, L. M. F., Zhou, J. & Laezza, F. Design, Synthesis, and Pharmacological Evaluation of Analogues Derived from the PLEV Tetrapeptide as Protein-Protein Interaction Modulators of Voltage-Gated Sodium Channel 1.6. Journal of medicinal chemistry 63, 11522-11547, doi:10.1021/acs.jmedchem.0c00531 (2020)
67 Wadsworth, P. A., Singh, A. K., Nguyen, N., Stephan, C. & Laezza, F. Bioluminescence Methodology for Ion Channel Studies. Methods Mol Biol 2188, 191-228, doi:10.1007/978-1-0716-0818-0_10 (2021)
68 Laezza, F. An interview with Fernanda Laezza, MD, PhD, by Vicki Glaser. Assay Drug Dev Technol 10, 105-110 (2012)
69 Dvorak, N. M., Wadsworth, P. A., Wang, P., Zhou, J. & Laezza, F. Development of Allosteric Modulators of Voltage-Gated Na(+) Channels: A Novel Approach for an Old Target. Curr Top Med Chem 21, 841-848, doi:10.2174/1568026621666210525105359 (2021). PubMed Central ID:PMCPMC8272397I.
70 Inglese, J., Johnson, R. L., Simeonov, A., Xia, M., Zheng, W., Austin, C. P. & Auld, D. S. High-throughput screening assays for the identification of chemical probes. Nat Chem Biol 3, 466-479 (2007)
71 Thorne, N., Inglese, J. & Auld, D. S. Illuminating insights into firefly luciferase and other bioluminescent reporters used in chemical biology. Chem Biol 17, 646-657 (2010)
72 Butkiewicz, M., Wang, Y., Bryant, S. H., Lowe, E. W., Jr., Weaver, D.

C. & Meiler, J. High-Throughput Screening Assay Datasets from the PubChem Database. Chem Inform 3PMC5962024 (2017). PubMed Central ID:PMCPMC5962024I.
73 Engelke, D. S., Zhang, X. O., O'Malley, J. J., Fernandez-Leon, J. A., Li, S., Kirouac, G. J., Beierlein, M. & Do-Monte, F. H. A hypothalamic-thalamostriatal circuit that controls approach-avoidance conflict in rats. Nat Commun 12, 2517, doi:10.1038/s41467-021-22730-y (2021). PubMed Central ID:PMCPMC8097010I.
74 Do-Monte, F. H., Manzano-Nieves, G., Quinones-Laracuente, K., Ramos-Medina, L. & Quirk, G. J. Revisiting the role of infralimbic cortex in fear extinction with optogenetics. J Neurosci 35, 3607-3615, doi:10.1523/jneurosci.3137-14.2015 (2015). PubMed Central ID:PMCPMC4339362I.
75 Do-Monte, F. H., Quinones-Laracuente, K. & Quirk, G. J. A temporal shift in the circuits mediating retrieval of fear memory. Nature 519, 460-463, doi:10.1038/nature14030 (2015). PubMed Central ID:PMCPMC4376623I.
76 Do-Monte, F. H., Minier-Toribio, A., Quinones-Laracuente, K., Medina-Colón, E. M. & Quirk, G. J. Thalamic Regulation of Sucrose Seeking during Unexpected Reward Omission. Neuron 94, 388-400.e384, doi:10.1016/j.neuron.2017.03.036 (2017). PubMed Central ID:PMCPMC5484638I.
77 Strovel, J., Sittampalam, S., Coussens, N. P., Hughes, M., Inglese, J., Kurtz, A., Andalibi, A., Patton, L., Austin, C., Baltezor, M., Beckloff, M., Weingarten, M. & Weir, S. in Assay Guidance Manual (eds S. Markossian et al.) (Eli Lilly & Company and the National Center for Advancing Translational Sciences, 2004).
78 Ali, S. R., Liu, Z., Nenov, M. N., Folorunso, O., Singh, A., Scala, F., Chen, H., James, T. F., Alshammari, M., Panova-Elektronova, N. I., White, M. A., Zhou, J. & Laezza, F. Functional modulation of voltage-gated sodium channels by a FGF14-based peptidomimetic. ACS Chem. Neurosci. 9, 976-987, doi:10.1021/acschemneuro.7b00399 (2018)
79 Dvorak, N. M., Wadsworth, P. A., Wang, P., Chen, H., Zhou, J. & Laezza, F. Bidirectional modulation of the voltage-gated sodium (Nav1.6) channel by rationally designed peptidomimetics. Molecules 25, 3365, doi:10.3390/molecules25153365 (2020). PubMed Central ID:PMCPMC7435778I.
80 Wang, P., Wadsworth, P. A., Dvorak, N. M., Singh, A. K., Chen, H., Liu, Z., Zhou, R., Holthauzen, L. M. F., Zhou, J. & Laezza, F. Design, Synthesis, and Pharmacological Evaluation of Analogues Derived from the PLEV Tetrapeptide as Protein-Protein Interaction Modulators of Voltage-Gated Sodium Channel 1.6. Journal of medicinal chemistry 63, 11522-11547, doi:10.1021/acs.jmedchem.0c00531 (2020)
81 Tetko, I. V., Gasteiger, J., Todeschini, R., Mauri, A., Livingstone, D., Ertl, P., Palyulin, V. A., Radchenko, E. V., Zefirov, N. S., Makarenko, A. S., Tanchuk, V. Y. & Prokopenko, V. V. Virtual computational chemistry laboratory—design and description. J Comput Aided Mol Des 19, 453-463, doi:10.1007/s10822-005-8694-y (2005)
82 Tetko, I. V. Computing chemistry on the web. Drug discovery today 10, 1497-1500, doi:10.1016/51359-6446(05)03584-1 (2005)
83 Pires, D. E., Blundell, T. L. & Ascher, D. B. pkCSM: Predicting Small-Molecule Pharmacokinetic and Toxicity Properties Using Graph-Based Signatures. Journal of medicinal chemistry 58, 4066-4072, doi:10.1021/acs.jmedchem.5b00104 (2015). PubMed Central ID:PMC4434528I.
84 Ali, S. R., Liu, Z., Nenov, M. N., Panova-Elektro, N. I., Zhou, J., Stoilova-McPhie, S. & Laezza, F. Targeting Protein:Protein Interaction Sites for Drug Development against Voltage-Gated Sodium Channels. Biophysical Journal 108, 574a, doi:10.1016/j.bpj.2014.11.3140 (2015)
85 Laezza, F., Lampert, A., Kozel, M. A., Gerber, B. R., Rush, A. M., Nerbonne, J. M., Waxman, S. G., Dib-Hajj, S. D. & Ornitz, D. M. FGF14N-terminal splice variants differentially modulate Nav1.2 and Nav1.6-encoded sodium channels. Mol Cell Neurosci 42, 90-101PMC2832592 (2009). PubMed Central ID:PMCPMC2832592I.
86 Clutterbuck, L. A., Posada, C. G., Visintin, C., Riddall, D. R., Lancaster, B., Gane, P. J., Garthwaite, J. & Selwood, D. L. Oxadiazolylindazole sodium channel modulators are neuroprotective toward hippocampal neurones. Journal of medicinal chemistry 52, 2694-2707 (2009)
87 Hille, B. Ionic channels: molecular pores of excitable membranes. Harvey Lect 82, 47-69 (1986)
88 Lou, J. Y., Laezza, F., Gerber, B. R., Xiao, M., Yamada, K. A., Hartmann, H., Craig, A. M., Nerbonne, J. M. & Ornitz, D. M. Fibroblast Growth Factor 14 is an Intracellular Modulator of Voltage-Gated Sodium Channels. J Physiol 569, 179-193 (2005)
89 Herzog, R. I., Liu, C., Waxman, S. G. & Cummins, T. R. Calmodulin binds to the C terminus of sodium channels Nav1.4 and Nav1.6 and differentially modulates their functional properties. J Neurosci 23, 8261-8270 (2003)
90 Gardill, B. R., Rivera-Acevedo, R. E., Tung, C. C. & Van Petegem, F. Crystal structures of Ca(2+)-calmodulin bound to Na(V)C-terminal regions suggest role for EF-hand domain in binding and inactivation. Proc Natl Acad Sci USA 116, 10763-10772, doi:10.1073/pnas.1818618116 (2019). PubMed Central ID:PMCPMC6561220I.
91 Gade, A. R., Marx, S. O. & Pitt, G. S. An interaction between the III-IV linker and CTD in NaV1.5 confers regulation of inactivation by CaM and FHF. J Gen Physiol 152, doi:10.1085/jgp.201912434 (2020). PubMed Central ID:PMCPMC7062510I.
92 Chichili, V. P., Xiao, Y., Seetharaman, J., Cummins, T. R. & Sivaraman, J. Structural Basis for the Modulation of the Neuronal Voltage-Gated Sodium Channel NaV1.6 by Calmodulin. Sci Rep 3, 2435PMC3743062 (2013). PubMed Central ID:PMCPMC37430621.
93 Ahern, C. A. What activates inactivation?J Gen Physiol 142, 97-100, doi:10.1085/jgp.201311046 (2013). PubMed Central ID:PMCPMC3727303I.
94 Nguyen, H. M. & Goldin, A. L. Sodium channel carboxyl-terminal residue regulates fast inactivation. J Biol Chem 285, 9077-9089PMC2838328 (2010). PubMed Central ID:PMCPMC2838328I.
95 Wisedchaisri, G., Tonggu, L., McCord, E., El-Din, T. M. G., Wang, L., Zheng, N. & Catterall, W. A. Resting-State structure and gating mechanism of a Voltage-Gated sodium channel. Cell 178, 993-1003. e1012 (2019)
96 Ritter, A. M., Ritchie, C. & Martin, W. J. Relationship between the firing frequency of injured peripheral neurons and inhibition of firing by sodium channel blockers. J Pain 8, 287-295 (2007)
97 Chen, H., Yang, Z., Ding, C., Chu, L., Zhang, Y., Terry, K., Liu, H., Shen, Q. & Zhou, J. Fragment-based drug design and identification of HJC0123, a novel orally bioavailable STAT3 inhibitor for cancer therapy. European journal of medicinal chemistry 62, 498-507, doi:10.1016/j.ejmech.2013.01.023 (2013). PubMed Central ID:PMC3750725I.
98 Chen, H., Zhou, X., Wang, A., Zheng, Y., Gao, Y. & Zhou, J. Evolutions in fragment-based drug design: the deconstruction-reconstruction approach. Drug discovery today 20, 105-113, doi:10.1016/j.drudis.2014.09.015 (2015). PubMed Central ID:PMC4305461I.
99 Bissaro, M., Sturlese, M. & Moro, S. The rise of molecular simulations in fragment-based drug design (FBDD): an overview. Drug discovery today, doi:10.1016/j.drudis.2020.06.023 (2020). PubMed Central ID:PMCPMC7314695I.
100 Wang, Q., Bardgett, M. E., Wong, M., Wozniak, D. F., Lou, J., McNeil, B. D., Chen, C., Nardi, A., Reid, D. C., Yamada, K. & Ornitz, D. M. Ataxia and paroxysmal dyskinesia in mice lacking axonally transported FGF14. Neuron 35, 25-38, doi:10.1016/s0896-6273(02)00744-4 (2002)
101 Gallo, E. F., Meszaros, J., Sherman, J. D., Chohan, M. O., Teboul, E., Choi, C. S., Moore, H., Javitch, J. A. & Kellendonk, C. Accumbens dopamine D2 receptors increase motivation by decreasing inhibitory transmission to the ventral pallidum. Nat Commun 9, 1086, doi:10.1038/s41467-018-03272-2 (2018). PubMed Central ID:PMCPMC5852096I.
102 Donnelly, N. A., Holtzman, T., Rich, P. D., Nevado-Holgado, A. J., Fernando, A. B., Van Dijck, G., Holzhammer, T., Paul, O., Ruther, P. & Paulsen, O. Oscillatory activity in the medial prefrontal cortex and nucleus accumbens correlates with impulsivity and reward outcome. PloS one 9, el 11300 (2014)
103 Fernandez-Leon, J. A., Engelke, D. S., Aquino-Miranda, G., Goodson, A., Rasheed, M. N. & Do Monte, F. H. Neural correlates and determinants of approach-avoidance conflict in the prelimbic prefrontal cortex. Elife 10, doi:10.7554/eLife.74950 (2021)
104 Nieh, E. H., Matthews, G. A., Allsop, S. A., Presbrey, K. N., Leppla, C. A., Wichmann, R., Neve, R., Wildes, C. P. & Tye, K. M. Decoding neural circuits that control compulsive sucrose seeking. Cell 160, 528-541, doi:10.1016/j.cell.2015.01.003 (2015). PubMed Central ID:PMCPMC4312417I.
105 Lima, S. Q., Hromidka, T., Znamenskiy, P. & Zador, A. M. PINP: a new method of tagging neuronal populations for identification during in vivo electrophysiological recording. PloS one 4, e6099, doi:10.1371/journal.pone.0006099 (2009). PubMed Central ID:PMCPMC2702752I.

1. A compound of Formula I or a pharmaceutically acceptable salt thereof, wherein: 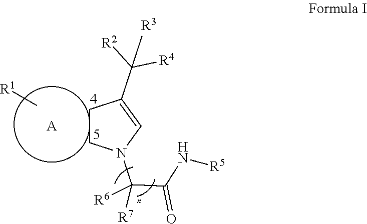
R¹ is H, alkyl, alkoxy, halogen, cyano, amino, hydroxyl, NO₂, CF₃ or —OCF₃;
A is an aryl ring or a heteroaryl ring (e.g., pyridine), wherein ring A is fused to the C4 and C5 of the 5-membered N-heterocycle ring moiety of Formula I, e.g., as shown the compounds exemplified below;
R² is H, alkyl, alkoxy, halogen, —CO₂R¹⁰, —CO₂Me or hydroxyl;
R³ is H, alkyl, alkoxy, halogen or hydroxyl;
or R² and R³ together form a 3-6 membered cycloalkyl ring;
R⁴ is H, alkyl, alkoxy, halogen, cyano, hydroxyl or NT¹T²;
T¹ is H, alkyl;
T² is independently chosen from: H, alkyl, cycloalkyl, benzyl, allyl, hydroxyl-alkyl;
or T¹ and T² together form a 4-12 membered cycloalkyl ring or cycloheteroalkyl ring, wherein the 4-12 membered cycloalkyl ring or cycloheteroalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
n=0, 1, 2, 3, or 4;
R⁵ is H, alkyl, aryl, cycloalkyl (e.g., is a bridged 6-14-membered bicyclic cycloalkyl, with 1-3 carbon length “bridge”); or heteroaryl, wherein each ring is optionally substituted with one or more groups selected independently from: H, alkyl, cycloalkyl, alkenyl, aryl, heteroaryl, benzyl, alkoxy (e.g., OMe, 3-OMe), halogen, cyano (e.g., 3-CN), nitro, amino, amido (e.g., 3-amido hydroxyl, —COOR⁸ (e.g., —COOMe), —CONHR⁹, CHF₂, CF₃ or —OCF₃, wherein alkyl is optionally substituted with one or more substituents chosen from: hydroxyl, cyan, amino, or halogen;
R⁶, and R⁷ are independently chosen from H, alkyl, F, CHF₂, CF₃, etc., or R⁶ and R⁷ taken together form a 3-7 membered ring (e.g., cyclopropane);
R⁸ is independently chosen from: H, alkyl, aryl, cycloalkyl, benzyl; and
R⁹ is independently chosen from: H, alkyl, aryl, cycloalkyl, benzyl, or substituted benzyl.
R¹⁰ is alkyl (e.g., C1-C6-alkyl), aryl, or alkylaryl.

2. (canceled)

3. (canceled)

4. The compound of claim 1, wherein R² and R³ are H.

5. The compound of claim 1, wherein R⁴ is OH.

6. (canceled)

7. (canceled)

8. The compound of claim 1, wherein: R¹, R² and R³ are H.

9. The compound of claim 1, wherein A is the aryl ring or pyridyl.

10. The compound of claim 1, wherein the compound structure is any one of the following:

| Compound | Structure |
|---|---|
| PW0999 | 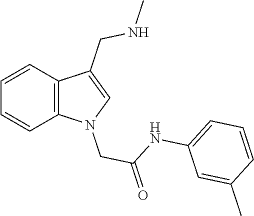 [SMILES: CNCc1cn(CC(=O)Nc2cccc(C)c2)c2ccccc12] |
| PW09100 | 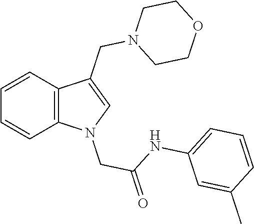 [SMILES: Cc1cccc(NC(=O)Cn2cc(CN3CCOCC3)c3ccccc32)c1] |
| PW01001 | 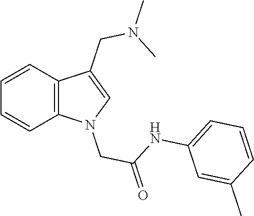 [SMILES: Cc1cccc(NC(=O)Cn2cc(CN(C)C)c3ccccc32)c1] |
| PW01002 | 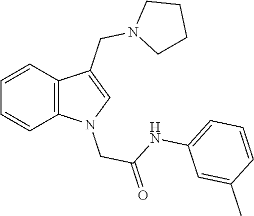 [SMILES: Cc1cccc(NC(=O)Cn2cc(CN3CCCC3)c3ccccc32)c1] |
| PW01003 | 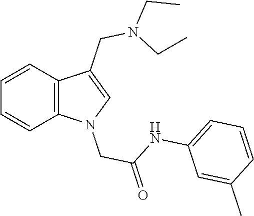 [SMILES: CCN(CC)Cc1cn(CC(=O)Nc2cccc(C)c2)c2ccccc12] |
| PW01004 | 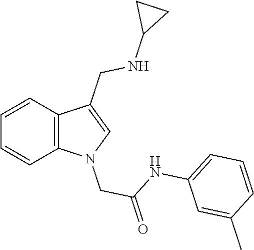 [SMILES: Cc1cccc(NC(=O)Cn2cc(CNC3CC3)c3ccccc32)c1] |
| PW01005 | 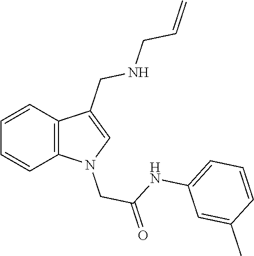 [SMILES: C=CCNCc1cn(CC(=O)Nc2cccc(C)c2)c2ccccc12] |
| PW01006 | 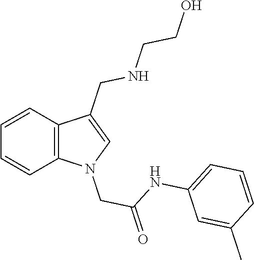 [SMILES: Cc1cccc(NC(=O)Cn2cc(CNCCO)c3ccccc32)c1] |
| PW01007 | 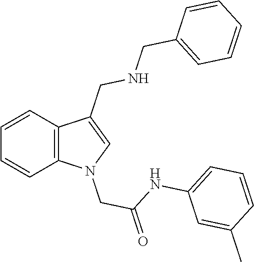 [SMILES: Cc1cccc(NC(=O)Cn2cc(CNCc3ccccc3)c3ccccc32)c1] |
| PW01011 | 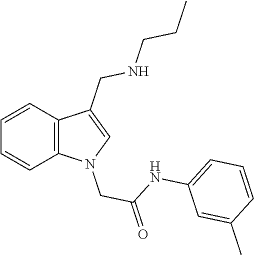 [SMILES: CCCNCc1cn(CC(=O)Nc2cccc(C)c2)c2ccccc12] |
| PW01016 | 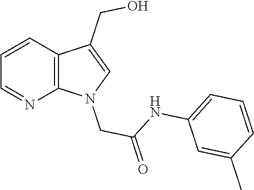 [SMILES: Cc1cccc(NC(=O)Cn2cc(CO)c3cccnc32)c1] |
| PW01017 | 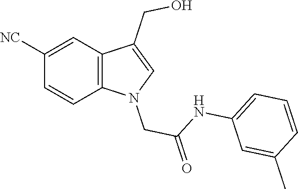 [SMILES: Cc1cccc(NC(=O)Cn2cc(CO)c3cc(C#N)ccc32)c1] |
| PW01020 | 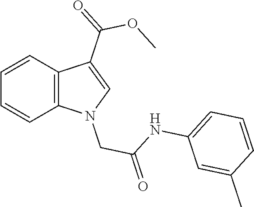 [SMILES: COC(=O)c1cn(CC(=O)Nc2cccc(C)c2)c2ccccc12] |
| PW01022 | 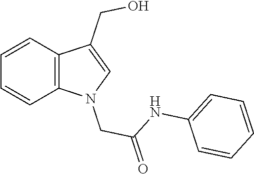 [SMILES: O=C(Cn1cc(CO)c2ccccc21)Nc1ccccc1] |
| PW01023 | 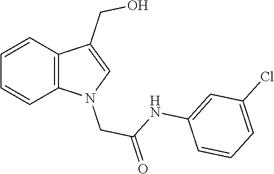 [SMILES: O=C(Cn1cc(CO)c2ccccc21)Nc1cccc(Cl)c1] |
| PW01024 | 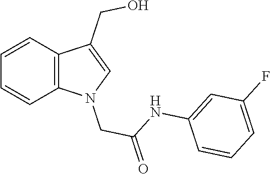 [SMILES: O=C(Cn1cc(CO)c2ccccc21)Nc1cccc(F)c1] |
| PW01025 | 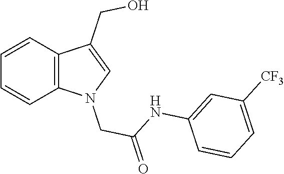 [SMILES: O=C(Cn1cc(CO)c2ccccc21)Nc1cccc(C(F)(F)F)c1] |
| PW01026 | 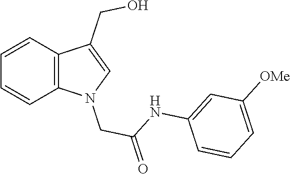 [SMILES: COc1cccc(NC(=O)Cn2cc(CO)c3ccccc32)c1] |
| PW01027 | 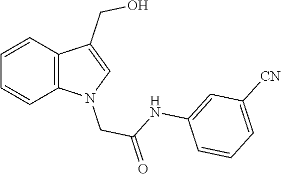 [SMILES: N#Cc1cccc(NC(=O)Cn2cc(CO)c3ccccc32)c1] |
| PW01028 | 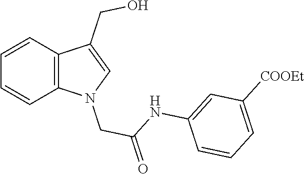 [SMILES: CCOC(=O)c1cccc(NC(=O)Cn2cc(CO)c3ccccc32)c1] |
| PW01029 | 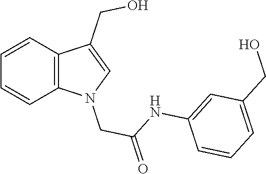 [SMILES: O=C(Cn1cc(CO)c2ccccc21)Nc1cccc(CO)c1] |
| PW01030 | 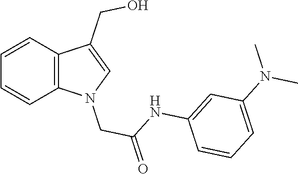 [SMILES: CN(C)c1cccc(NC(=O)Cn2cc(CO)c3ccccc32)c1] |
| PW01031 | 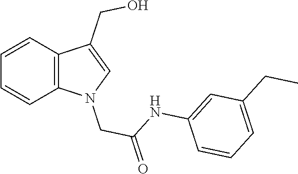 [SMILES: CCc1cccc(NC(=O)Cn2cc(CO)c3ccccc32)c1] |
| PW01032 | 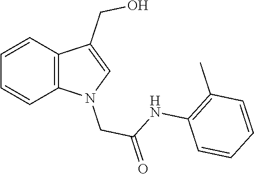 [SMILES: Cc1ccccc1NC(=O)Cn1cc(CO)c2ccccc21] |
| PW01033 | 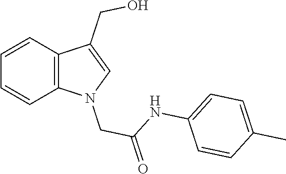 [SMILES: Cc1ccc(NC(=O)Cn2cc(CO)c3ccccc32)cc1] |
| PW01034 | 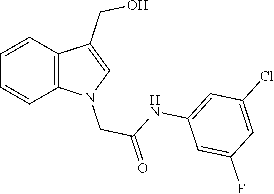 [SMILES: O=C(Cn1cc(CO)c2ccccc21)Nc1cc(F)cc(Cl)c1] |
| PW01035 | 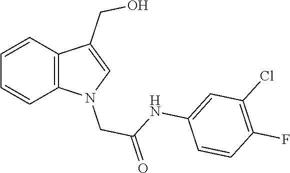 [SMILES: O=C(Cn1cc(CO)c2ccccc21)Nc1ccc(F)c(Cl)c1] |
| PW01036 | 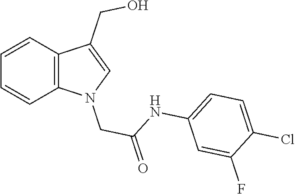 [SMILES: O=C(Cn1cc(CO)c2ccccc21)Nc1ccc(Cl)c(F)c1] |
| PW01037 | 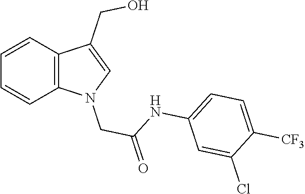 [SMILES: O=C(Cn1cc(CO)c2ccccc21)Nc1ccc(C(F)(F)F)c(Cl)c1] |
| PW01038 | 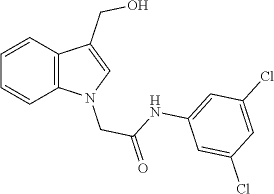 [SMILES: O=C(Cn1cc(CO)c2ccccc21)Nc1cc(Cl)cc(Cl)c1] |
| PW01039 | 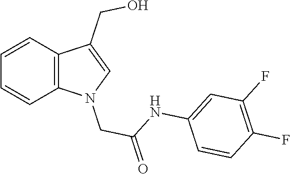 [SMILES: O=C(Cn1cc(CO)c2ccccc21)Nc1ccc(F)c(F)c1] |
| PW01040 | 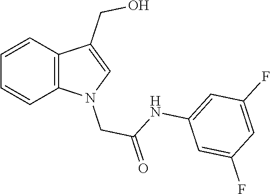 [SMILES: O=C(Cn1cc(CO)c2ccccc21)Nc1cc(F)cc(F)c1] |
| PW01041 | 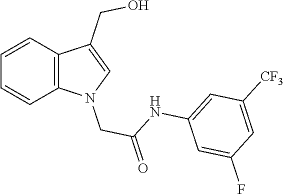 [SMILES: O=C(Cn1cc(CO)c2ccccc21)Nc1cc(F)cc(C(F)(F)F)c1] |
| PW01042 | 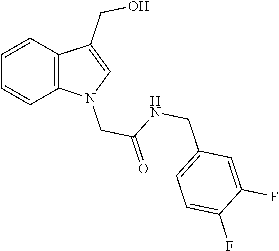 [SMILES: O=C(Cn1cc(CO)c2ccccc21)NCc1ccc(F)c(F)c1] |
| PW01043 | 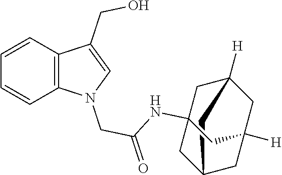 [SMILES: O=C(Cn1cc(CO)c2ccccc21)NC12C[C@H]3C[C@@H](C1)C[C@@H](C2)C3] |
| PW01044 | 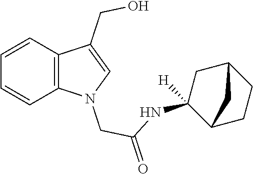 [SMILES: O=C(Cn1cc(CO)c2ccccc21)N[C@H]1C[C@@H]2CC[C@H]1C2] |
| PW01045 | 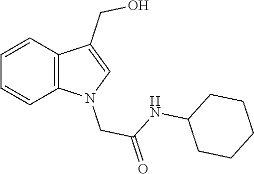 [SMILES: O=C(Cn1cc(CO)c2ccccc21)NC1CCCCC1] |
| PW01046 | 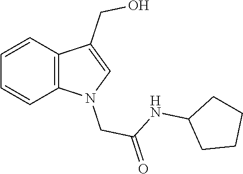 [SMILES: O=C(Cn1cc(CO)c2ccccc21)NC1CCCC1] |
| PW01050 | 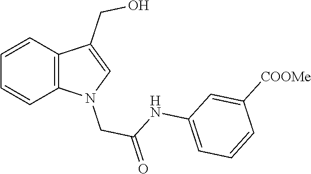 [SMILES: COC(=O)c1cccc(NC(=O)Cn2cc(CO)c3ccccc32)c1] |
| PW01051 | 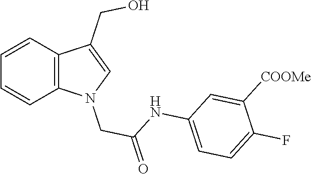 [SMILES: COC(=O)c1cc(NC(=O)Cn2cc(CO)c3ccccc32)ccc1F] |
| PW01053 | 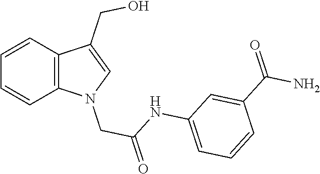 [SMILES: NC(=O)c1cccc(NC(=O)Cn2cc(CO)c3ccccc32)c1] |
| PW01061 | 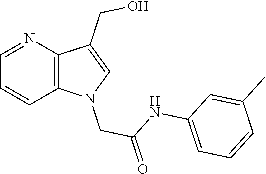 [SMILES: Cc1cccc(NC(=O)Cn2cc(CO)c3ncccc32)c1] |
| PW01062 | 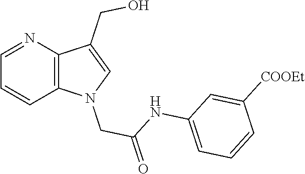 [SMILES: CCOC(=O)c1cccc(NC(=O)Cn2cc(CO)c3ncccc32)c1] |
| PW01072 | 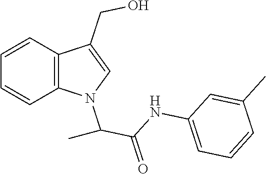 [SMILES: Cc1cccc(NC(=O)C(C)n2cc(CO)c3ccccc32)c1] |
| PW01073 | 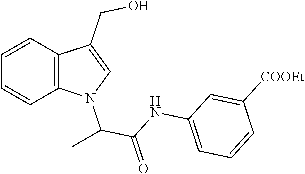 [SMILES: CCOC(=O)c1cccc(NC(=O)C(C)n2cc(CO)c3ccccc32)c1] |
| PW01074 | 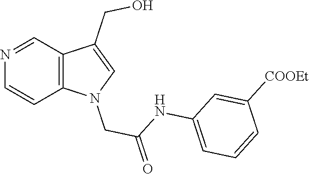 [SMILES: CCOC(=O)c1cccc(NC(=O)Cn2cc(CO)c3cnccc32)c1] |
| PW01076 | 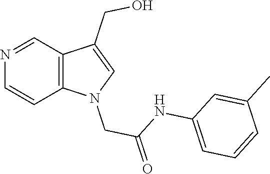 [SMILES: Cc1cccc(NC(=O)Cn2cc(CO)c3cnccc32)c1] |
| PW01083 | 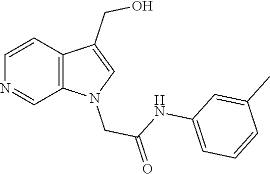 [SMILES: Cc1cccc(NC(=O)Cn2cc(CO)c3ccncc32)c1] |
| PW01084 | 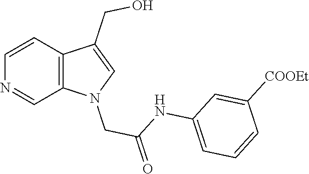 [SMILES: CCOC(=O)c1cccc(NC(=O)Cn2cc(CO)c3ccncc32)c1] |
| JY0137 | 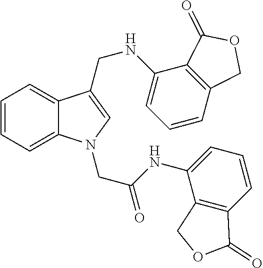 [SMILES: O=C(Cn1cc(CNc2cccc3c2C(=O)OC3)c2ccccc21)Nc1cccc2c1COC2=O] |
| JY0142 | 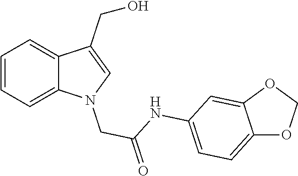 [SMILES: O=C(Cn1cc(CO)c2ccccc21)Nc1ccc2c(c1)OCO2] |
| JY0143 | 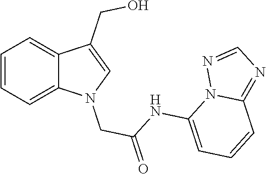 [SMILES: O=C(Cn1cc(CO)c2ccccc21)Nc1cccc2ncnn12] |
| JY0144 | 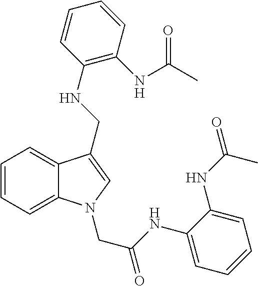 [SMILES: CC(=O)Nc1ccccc1NCc1cn(CC(=O)Nc2ccccc2NC(C)=O)c2ccccc12] |
| JY0145 | 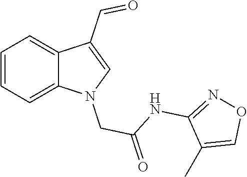 [SMILES: Cc1conc1NC(=O)Cn1cc(C=O)c2ccccc21] |
| JY0147 | 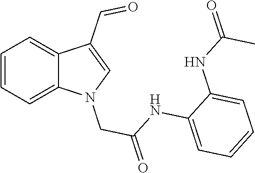 [SMILES: CC(=O)Nc1ccccc1NC(=O)Cn1cc(C=O)c2ccccc21] |
| JY0149 | 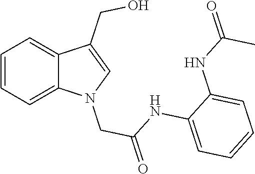 [SMILES: CC(=O)Nc1ccccc1NC(=O)Cn1cc(CO)c2ccccc21] |
| JY0151 | 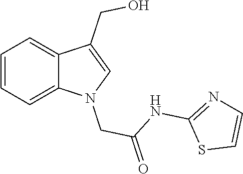 [SMILES: O=C(Cn1cc(CO)c2ccccc21)Nc1nccs1] |
| JY0153 | 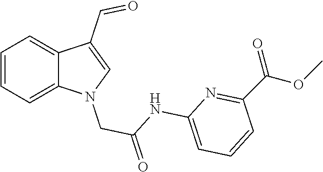 [SMILES: COC(=O)c1cccc(NC(=O)Cn2cc(C=O)c3ccccc32)n1] |
| JY0154 | 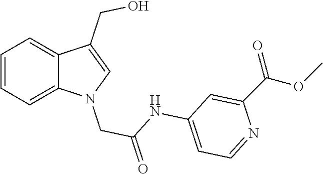 [SMILES: COC(=O)c1cc(NC(=O)Cn2cc(CO)c3ccccc32)ccn1] |
| JY0155 | 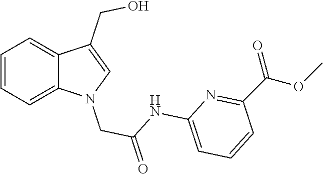 [SMILES: COC(=O)c1cccc(NC(=O)Cn2cc(CO)c3ccccc32)n1] |
| JY0156 | 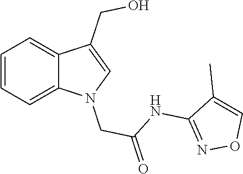 [SMILES: Cc1conc1NC(=O)Cn1cc(CO)c2ccccc21] |
| JY0159 | 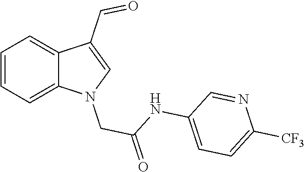 [SMILES: O=Cc1cn(CC(=O)Nc2ccc(C(F)(F)F)nc2)c2ccccc12] |
| JY0160 | 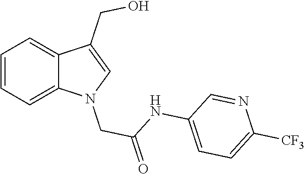 [SMILES: O=C(Cn1cc(CO)c2ccccc21)Nc1ccc(C(F)(F)F)nc1] |
| JY0161 | 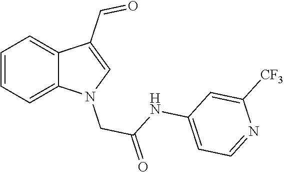 [SMILES: O=Cc1cn(CC(=O)Nc2ccnc(C(F)(F)F)c2)c2ccccc12] |
| JY0162 | 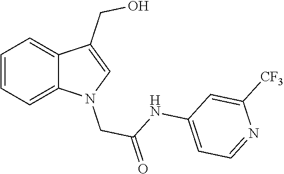 [SMILES: O=C(Cn1cc(CO)c2ccccc21)Nc1ccnc(C(F)(F)F)c1] |
| JY0163 | 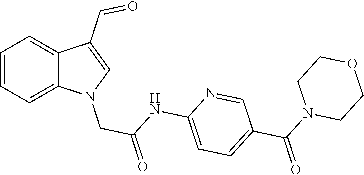 [SMILES: O=Cc1cn(CC(=O)Nc2ccc(C(=O)N3CCOCC3)cn2)c2ccccc12] |
| JY0164 | 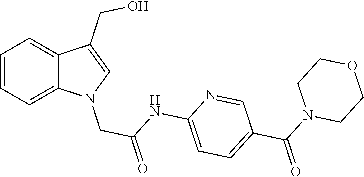 [SMILES: O=C(Cn1cc(CO)c2ccccc21)Nc1ccc(C(=O)N2CCOCC2)cn1] |
| JY0166 | 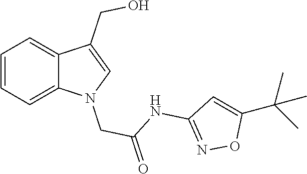 [SMILES: CC(C)(C)c1cc(NC(=O)Cn2cc(CO)c3ccccc32)no1] |
| JY0167 | 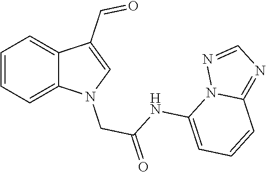 [SMILES: O=Cc1cn(CC(=O)Nc2cccc3ncnn23)c2ccccc12] |
| JY0168 | 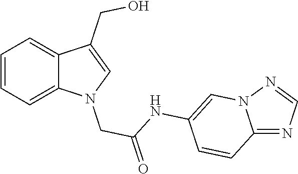 [SMILES: O=C(Cn1cc(CO)c2ccccc21)Nc1ccc2ncnn2c1] |
| JY0169 | 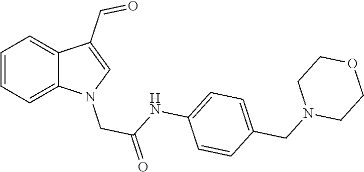 [SMILES: O=Cc1cn(CC(=O)Nc2ccc(CN3CCOCC3)cc2)c2ccccc12] |
| JY0170 | 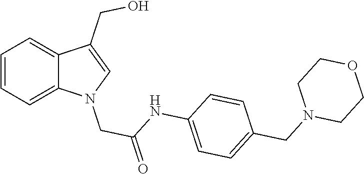 [SMILES: O=C(Cn1cc(CO)c2ccccc21)Nc1ccc(CN2CCOCC2)cc1] |
| JY0172 | 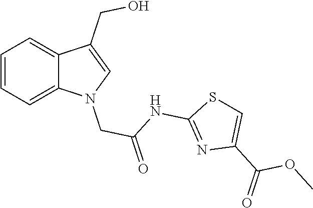 [SMILES: COC(=O)c1csc(NC(=O)Cn2cc(CO)c3ccccc32)n1] |
| JY0186 | 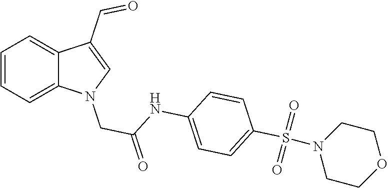 [SMILES: O=Cc1cn(CC(=O)Nc2ccc(S(=O)(=O)N3CCOCC3)cc2)c2ccccc12] |
| JY0187 | 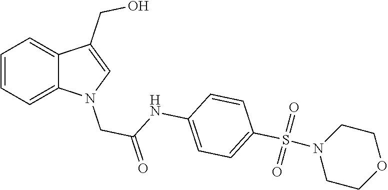 [SMILES: O=C(Cn1cc(CO)c2ccccc21)Nc1ccc(S(=O)(=O)N2CCOCC2)cc1] |
| JY0188 | 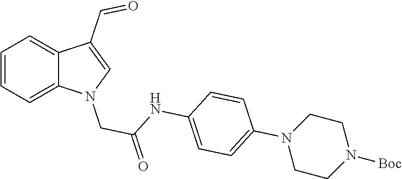 [SMILES: CC(C)(C)OC(=O)N1CCN(c2ccc(NC(=O)Cn3cc(C=O)c4ccccc43)cc2)CC1] |
| JY0189 | 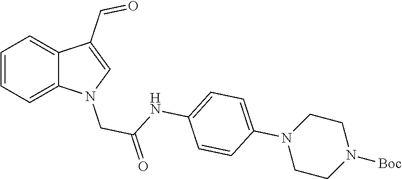 [SMILES: CC(C)(C)OC(=O)N1CCN(c2ccc(NC(=O)Cn3cc(C=O)c4ccccc43)cc2)CC1] |
| CZ01 | 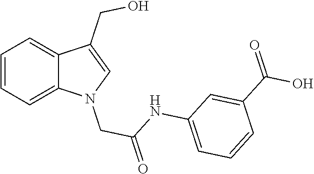 [SMILES: O=C(Cn1cc(CO)c2ccccc21)Nc1cccc(C(=O)O)c1] |
| CZ02 | 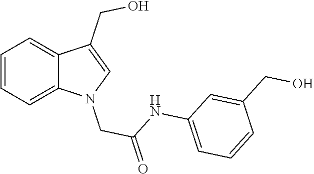 [SMILES: O=C(Cn1cc(CO)c2ccccc21)Nc1cccc(CO)c1] |
| CZ03 | 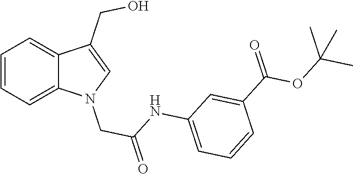 [SMILES: CC(C)(C)OC(=O)c1cccc(NC(=O)Cn2cc(CO)c3ccccc32)c1] |
| CZ04 | 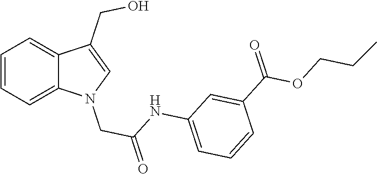 [SMILES: CCCOC(=O)c1cccc(NC(=O)Cn2cc(CO)c3ccccc32)c1] |
| CZ05 | 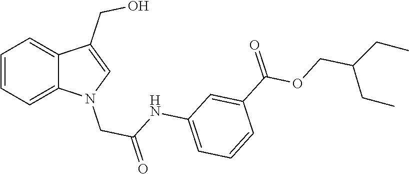 [SMILES: CCC(CC)COC(=O)c1cccc(NC(=O)Cn2cc(CO)c3ccccc32)c1] |
| CZ06 | 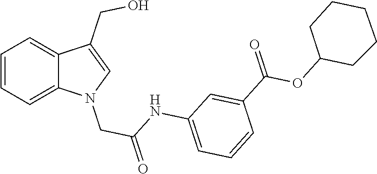 [SMILES: O=C(Cn1cc(CO)c2ccccc21)Nc1cccc(C(=O)OC2CCCCC2)c1] |
| CZ07 | 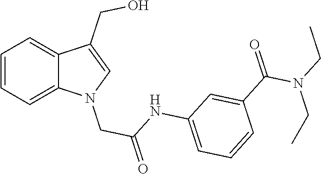 [SMILES: CCN(CC)C(=O)c1cccc(NC(=O)Cn2cc(CO)c3ccccc32)c1] |
| CZ08 | 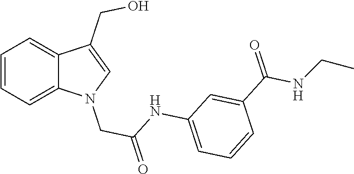 [SMILES: CCNC(=O)c1cccc(NC(=O)Cn2cc(CO)c3ccccc32)c1] |
| CZ09 | 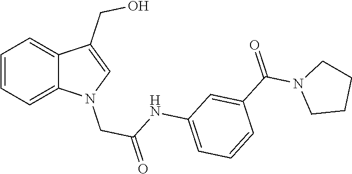 [SMILES: O=C(Cn1cc(CO)c2ccccc21)Nc1cccc(C(=O)N2CCCC2)c1] |
| CZ10 | 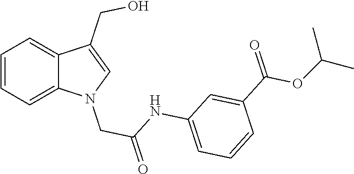 [SMILES: CC(C)OC(=O)c1cccc(NC(=O)Cn2cc(CO)c3ccccc32)c1] |
| CZ11 | 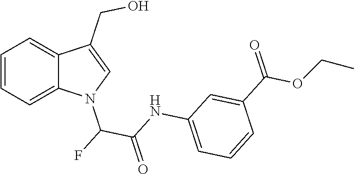 [SMILES: CCOC(=O)c1cccc(NC(=O)C(F)n2cc(CO)c3ccccc32)c1] |
| CZ12 | 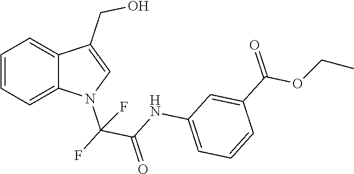 [SMILES: CCOC(=O)c1cccc(NC(=O)C(F)(F)n2cc(CO)c3ccccc32)c1] |
| CZ13 | 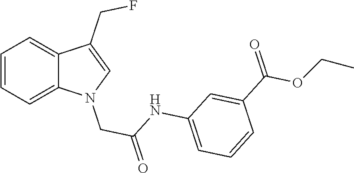 [SMILES: CCOC(=O)c1cccc(NC(=O)Cn2cc(CF)c3ccccc32)c1] |
| CZ14 | 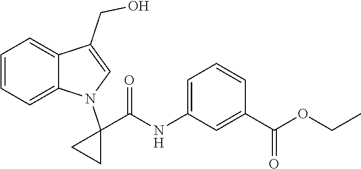 [SMILES: CCOC(=O)c1cccc(NC(=O)C2(n3cc(CO)c4ccccc43)CC2)c1] |
| CZ15 | 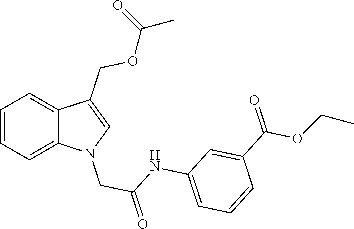 [SMILES: CCOC(=O)c1cccc(NC(=O)Cn2cc(COC(C)=O)c3ccccc32)c1] |
| CZ16 | 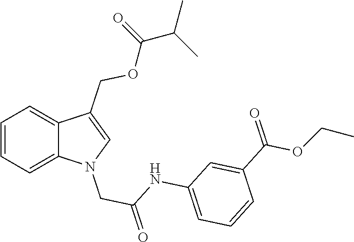 [SMILES: CCOC(=O)c1cccc(NC(=O)Cn2cc(COC(=O)C(C)C)c3ccccc32)c1] |
| CZ17 | 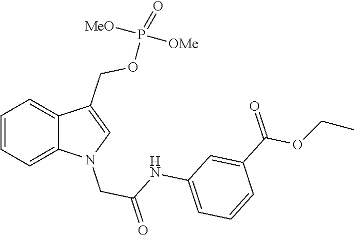 [SMILES: CCOC(=O)c1cccc(NC(=O)Cn2cc(COP(=O)(OC)OC)c3ccccc32)c1] |
| CZ18 | 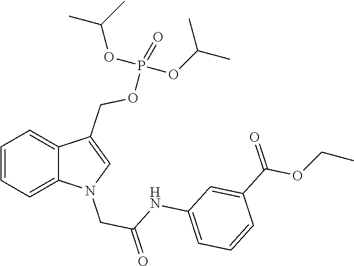 [SMILES: CCOC(=O)c1cccc(NC(=O)Cn2cc(COP(=O)(OC(C)C)OC(C)C)c3ccccc32)c1] |

11. A compound of the Formula (II) or a pharmaceutically acceptable salt thereof, wherein: 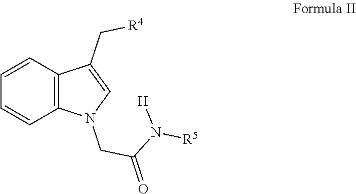
R⁴ is hydroxyl or NT¹T²; wherein T¹ is H or alkyl; and wherein T² is independently chosen from H, alkyl, cycloalkyl, benzyl, allyl, hydroxyl-alkyl;
or T¹ and T² together form a 4-12 membered cycloalkyl ring or cycloheteroalkyl ring, wherein the 4-12 membered cycloalkyl ring is optionally substituted with one or more groups selected independently from alkyl, cycloalkyl, alkenyl, aryl, heteroaryl, benzyl, alkoxy, halogen, cyan, nitro, amino, hydroxyl, CHF₂, CF₃ or —OCF₃;
R⁵ is an aryl ring or a cycloalkyl ring, wherein each ring is optionally substituted with one or more groups selected independently from: H, alkyl, cycloalkyl, alkenyl, aryl, heteroaryl, benzyl, alkoxy, halogen, cyano, nitro, amino, hydroxyl, —COOR⁸, —CONR⁹, CHF₂, CF₃ or —OCF₃, wherein alkyl is optionally substituted with one or more chosen substituents chosen from: hydroxyl, cyano, amino, or halogen;
R⁸ is independently chosen from: H, alkyl, aryl, cycloalkyl, or benzyl; and
R⁸ is independently chosen from: H, alkyl, aryl, cycloalkyl, benzyl, or substituted benzyl.

12. The compound of claim 11, wherein R⁴ is hydroxyl.

13. (canceled)

14. (canceled)

15. The compound of claim 11, wherein R⁵ is an aryl ring.

16. The compound of claim 15, wherein the aryl ring is substituted with one or more substituents selected independently from: H, alkyl, benzyl, alkoxy, halogen, cyano, nitro, amino, hydroxyl, —COOR⁸, —CONR⁹, CHF₂, CF₃ or —OCF₃, wherein alkyl is optionally substituted with one or more chosen substituents chosen from: hydroxyl, cyano, amino, or halogen;
R⁸ is H, alkyl; and
R⁹ is independently chosen from: H, alkyl, aryl, cycloalkyl, benzyl, or substituted benzyl.

17. (canceled)

18. (canceled)

19. (canceled)

20. (canceled)

21. The compound of formula (II), or a pharmaceutically acceptable salt thereof, wherein: 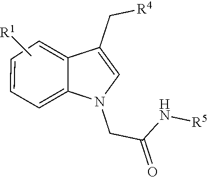
wherein R¹, R⁴, and R⁵ are defined as for the compound of Formula I.

22. The compound of formula (II), or a pharmaceutically acceptable salt thereof, wherein: 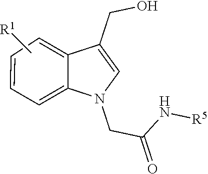
wherein R¹ and R⁵ are defined as for the compound of Formula I.

23. (canceled)

24. (canceled)

25. (canceled)

26. A method of treating a disease or condition in a patient comprising administering to the patient a therapeutically effective amount of a compound of any of the preceding claims or a pharmaceutically acceptable salt thereof.

27. The method of claim 26, wherein treatment of the disease or condition involves modulating a FGF14:NAV1.6 channel complex protein-protein interaction.

28. The method of claim 26, wherein said compound modulates voltage-gated Na+(Nav) channels and their isoforms (such as Nav1.6).

29. The method of claim 26, wherein said compound modulates voltage-gated sodium channels and their accessory regulator proteins complex protein-protein interactions.

30. The method of claim 26, wherein said compound modulates voltage-gated sodium channel accessory regulator proteins (such as intracellular fibroblast growth factors (e.g., FGF14, FGF13, FGF12 and FGF11)).

31. The method of claim 26, said compound modulates FGF14:Nav1.6 channel complex formation and increases Nav1.6 channel availability (and medium spiny neuron (MSN) firing).

32. (canceled)

33. The method of claim 26, wherein the disease or condition is a central nervous system chosen from any of brain disorders, neurological disorders, psychiatric disorders, substance abuse disorders, neuroinflammation, or pain.

34. The method of claim 26, wherein the compound of Formula (I) is compound PW0989.
